(19) United States
(12) Patent Application Publication
(10) Pub. No.: US 2007/0141072 A1
(43) Pub. Date: Jun. 21, 2007

(54) Use of multifunctional transcription factor yin-yang-1 and variants thereof for treating illnesses, especially type 1 diabetes

(75) Inventors: Ingrid Kloting, Luhmannsdorf (DE); Nora Kloting, Luhmannsdorf (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: ERNST-MORITZ-ARNDT-UNIVERSITAT GREIFSWALD, Greifswald (DE)

(21) Appl. No.: 10/539,535

(22) PCT Filed: Dec. 19, 2003
(86) PCT No.: PCT/EP03/14762

§ 371 Date: Jun. 19, 2006

(30) Foreign Application Priority Data
Dec. 20, 2002 (DE) 10261650.7

(51) Int. Cl. A61K 39/00; C07H 21/04; C12P 21/06; C07K 16/28; C07K 14/705

(52) U.S. Cl. 424/185.1; 435/69.1; 435/320.1; 435/325; 530/350; 536/23.5

(57) ABSTRACT

The present invention relates to the influence of the activity and/or the expression of the multifunctional transcription factor Yin Yang 1 (YY1), and of variants thereof, for the treatment of many different illnesses. The invention especially relates to a mutated nucleic acid sequence which encodes a variant of the human YY1 and has a protective action against diabetes, according to the present invention.

The present invention relates to influencing the activity and/or the expression of the multifunctional transcription factor Yin Yang 1 (YY1), and of variants thereof, for the treatment of many different illnesses. The invention especially relates to a mutated nucleic acid sequence which encodes a variant of the human YY1 and has a protective action against diabetes, according to the present invention.

### INTRODUCTION

Insulin-dependent type 1 diabetes (IDDM—Insulin-Dependent Diabetes Mellitus) is a complex illness in which cellular and humoral immune processes concur to result in destruction of the body's own insulin-producing beta cells. This is why we talk of autoimmune disease in the case of type 1 diabetes. Today it is beyond all further doubt that type 1 diabetes is genetically determined. It is known for certain that certain class II genes of the major histocompatibility complex (MHC), termed HLA (Human Leucocyte Antigens) in the case of humans, play a part in the formation of type 1 diabetes, but are not the sole factors. These diabetes-susceptible class II genes, termed IDDM1, only account for about 40% of the risk of falling ill with type 1 diabetes. This is why in recent years attempts have been made to localise chromosomal regions with diabetogenic non-MHC genes. As a result of these studies, more than 17 non-MHC genes (IDDM2, IDDM3, IDDM4 etc.) have so far been described, mapping over various different chromosomes. With the exception of IDDM2 and IDDM18, where the diabetogenic gene is known, the relevant genes for the other IDDMs are still unknown (1,2).

In the identification of these non-MHC genes, animal models are of great help, where there is development of type 1 diabetes similar to humans. The spontaneously diabetic BB/O(ttawa)K(arlsburg) rat is superbly suited to animal modelling for type 1 diabetes, since this animal model displays extensive analogies to human type 1 diabetes both clinically and etiopathogenically (3-7). In the case of the BB/OK rat too, class II genes of the MHC are essential but not sufficient for development of diabetes. The BB/OK rat is homozygous for the MHC RT1^{u} haplotype. As in the case of humans, this diabetogenic gene is called Iddm1. Along with this gene complex, there is however another gene, Iddm2, needed for development of diabetes in the case of the BB/OK rat. This is a gene (1) that causes lymphopenia, and which is inherited recessively and termed Iddm2. The fact that both diabetogenic genes, Iddm1 and Iddm2, are indeed essential but not sufficient, has been demonstrated by various interbreeding studies using diabetic BB/OK rats and various diabetes resistant strains of rat (8-11). It is of no matter whether diabetes resistant LEW.1A, DA, SHR or wild rats (12) are interbred with BB/OK rats—in all cases no F1 hybrid resulted and only about 50% of the first backcross hybrids (R1) that were already homozygous for both the Iddm1 and Iddm2 genes developed type 1 diabetes. The percentage of 50% diabetic animals for the R1 hybrids that are already homozygous as regards Iddm1 and Iddm2 indicates that at least one more diabetogenic gene, Iddm3, is needed for diabetes to develop in BB/OK rats. A genomic search for this third gene in two interbred populations, [(BB×DA)F1×BB] and [(BB×SHR)F1×BB], has shown that this “third gene” is not just one gene. Two diabetogenic genes, Iddm3 and Iddm4, were mapped on chromosome 18 and 6 respectively, and one diabetes protective gene, Iddm5r, on chromosome 1 (13, 14). Whereas Iddm3 has been demonstrated in both interbred populations and thereby confirmed (13), Iddm4 and Iddm5r could only be demonstrated for [(BB×SHR)F1'BB] R1 hybrids (14).

If a comparison is made between the homologous aspects for rats and humans, it turns out that for humans the homologous regions map as follows: Iddm3 on 18q21-q23, Iddm4 on 14q24-q32 and Iddm5r on 11p15. These are areas where for humans too there was similar mapping of the following diabetogenic genes: IDDM6 on 18q21-23 (Iddm3), IDDM11 on 14q24-q32 (Iddm4) and IDDM2 on 11p15 (Iddm5r)(15-17).

The task of the present invention is therefore in the first instance to identify the diabetogenic genes and formulate therapeutic approaches for the prevention of type 1 diabetes.

As part of the invention it came as a surprise to identify a gene that is in the diabetes protective chromosomal region which mutated variants are suited for prevention of type 1 diabetes in the rat model. As a result of these findings, for the first time an important therapeutic approach has become available for the diagnosis and preventative therapy of type 1 diabetes. In addition, many additional application possibilities described below result from further findings acquired according to the present invention.

Preparatory Work

To check the actual significance of the mapped Iddms of the BB/OK rat with regard to development of type 1 diabetes, various congenic BB/OK rat lines were established.

Along with the congenic BB.SHR lines already described (BB.1K, BB.Sa, BB.LL, BB.Bp2, BB.Xs) two further BB.SHR lines were established (18-24). One region of chromosome 6 (D6Rat184-Iddm4-D6Rat3, approx. 15 cM) and one of 18 (Olf-Iddm3-D18Rat44, approx. 24 cM) of the BB/OR rat was replaced by that of the SHR rats (24). The phenotypic characterisation of these newly established BB lines, abbreviated to BB.6S and BB.18S showed that the incidence of diabetes in both congenes could be reduced, in particular, however, for the BB.6S line congenes (24). Whereas around 86% of all BB/OK rats develop type 1 diabetes by their 32^{nd} week of life, within the same period only 14% of the newly established BB.6S line succumbed and this in spite of the fact that the BB.6S animals are homozygous with regard to Iddm1 and Iddm2 and correspond to the BB/OK rat for the remaining genome, as confirmed in genomic analysis (24). In other words the effect of both essential diabetogenic genes, Iddm1 and Iddm2, is almost completely suppressed by one or more gene(s) in the transferred region of the diabetes resistant SHR rat. This result is unique to date. Even in the case of the N(on)O(bese)D(iabetic) mouse, which is also a model for type 1 diabetes, diabetogenic genes have been mapped and analogously congenic NOD lines established to examine the in vivo effect of these genes. No drastic reduction in the incidence of diabetes resulting from exchange of a diabetogenic chromosomal region, as with BB.6S, has hitherto been described. A similar clear reduction in the incidence of diabetes as with BB.6S has only been achieved by exchanging 2 or more chromosomal regions of the NOD with the diabetes resistant strains (25-28).

Differences between BB/OK and BB.6S rats have also been demonstrated with regard to the age of manifestation and the lymphocyte phenotypes. Manifestation in BB.6S rats occurs significantly later (137±14 as against 103±30 days, p<0.001) and these rats have significantly fewer activated T lymphocytes than BB/OK rats (36.6±6.9 as against 65.6±18.4%, p<0.0001) (24). In addition, BB.6S rats become significantly heavier and have significantly higher serum cholesterol readings than BB/OK rats (24).

Given that for the SHR rat too, immunological phenomena have been described (29-31), the drastic reduction in the incidence of diabetes from 86% for BB/OK to 14% for BB.6S was initially interpreted as due to one or more SHR gene(s) being located in the transferred region whose product(s) can to a large extent “neutralise” the autoaggression of the diabetogenic gene products of the BB rat. This assumption was underlined by the establishment of another congenic BB/OK rat line.

Along with diabetes resistant SHR rats, wild rats were also used for breeding congenic BB.K(arlsburg)W(ild)R(at)rats. Analogously to the BB.6S line, the same chromosomal region of the BB/OK of 15 cM was exchanged with that of wild rats(BB.6W). The incidence of disease in this congenic BB.6W line is comparable to that of the BB/OK rat (89% as against 86%, 32). This is why in accordance with the present invention it has been assumed that diabetes protection for BB.6S results from the “neutralising” gene effect of the SHR rat as opposed to being due to replacing Iddm4 of the BB/OK rat.

This meant that identification of the gene(s) was needed, since the possibility of undertaking gene-specific treatment and/or prevention of type 1 diabetes or autoimmune diseases per se required identification of this/these gene(s).

Continued Work on Identifying the Diabetes Protective Gene(s)

After decoding of the human genome (approximately 40,000 genes), it is nowadays assumed that there are about 15-20 genes per cM and not 50 as hitherto supposed. Based on this assumption, there should be mapping of about 225-300 genes in the transferred chromosomal region of the BB.6S rat of approximately 15 cM. In view of this figure, identification of possible candidate genes for diabetes protection is a hopeless undertaking, and this is why subcongenic BB.6S rats were bred to further narrow down the chromosomal region that has a diabetes protective effect.

BB.6S rats were interbred with diabetic BB/OK rats. The resultant F1 hybrids for the region of chromosome 6 were mated with each other (intercross) and genetically analysed, with the marker spectrum being extended to allow recognition of any recombinations forthcoming in small regions: D6Rat3-D6Rat1-D6wox13-D6Rat101-Ighe/Ckb-D6Mgh2-D6Rat94-D6Rat183-D6Rat10-D6Rat7-D6Rat75-D6Rat9-D6Rat6-D6Rat160-D6Mgh9-D6Rat184.

Assignment of the lines with diabetes protection was based on incidence of disease. If in excess of 50% of the descendents of a subcongenic line fell ill before their 100^{th} day, then this phenotype indicated that of the BB/OK rat and the line was eliminated. If less than 15% fell ill by the 100^{th} day, then this line indicated the BB.6S phenotype. These lines were used for further breeding with the intention of minimising the diabetes protective region. By establishing various subcongenic lines (BB.6Sa, b, c . . . ) with the phenotype of the BB.6S rat, the relevant chromosomal region was narrowed down to <2 cM (30-40 genes).

As illustrated below, the diabetes protective gene is mapped around the locus D6Mgh2.

TABLE 1

| cM | | BB.6S | BB.6a | BB.6b | BB.6c | BB.6d | BB.6e | BB.6f |
|---|---|---|---|---|---|---|---|---|
| | D6Mgh4 | BB | BB | BB | BB | BB | BB | BB |
| 2.1 | D6Rat13 | BB | BB | BB | BB | BB | BB | BB |
| 4.4 | D6Wox5 | BB | BB | BB | BB | BB | BB | BB |
| 1.5 | D6Rat66 | BB | BB | BB | BB | BB | BB | BB |
| 2.0 | D6Rat184/D6Mgh9 | SHR | BB | BB | BB | BB | BB | SHR |
| 0.8 | D6Rat160 | SHR | BB | BB | BB | BB | BB | SHR |
| 1.3 | D6Rat6 | SHR | BB | BB | BB | BB | BB | SHR |
| | D6Rat9 | SHR | BB | BB | BB | BB | BB | SHR |
| 0.4 | D6Rat75 | SHR | | | | | | |
| 0.4 | D6Rat7 | SHR | BB | BB | BB | BB | BB | SHR |
| 2.1 | D6Rat10 | SHR | BB | BB | BB | BB | BB | SHR |
| 1.6 | D6Rat183 | SHR | BB | SHR | BB | BB | BB | SHR |
| 0.4 | D6Rat94 | SHR | BB | SHR | BB | BB | BB | SHR |
| 1.9 | D6Mgh2 | SHR | SHR | SHR | BB | BB | BB | SHR |
| 1.7 | Ighe | SHR | SHR | BB | SHR | SHR | SHR | SHR |
| | Ckb/D6Rat101 | SHR | SHR | BB | SHR | SHR | BB | SHR |
| 1.0 | D6Rat1 | SHR | SHR | BB | BB | BB | SHR | BB |
| 1.9 | D6Rat3 | SHR | SHR | BB | BB | SHR | SHR | BB |
| | Incidence of | 15% | 10% | 22% | >50% | >50% | >50% | 14% |
| | diabetes | | | | | | | |

Locus D6Mgh2 is a microsatellite in the Ef1 gene. Ef1 is a pseudogene and in humans maps on chromosome 14q32 and in mice on chromosome 12q14. Based on the chromosome map of Watanabe et al. (33), the Macs gene should also map onto chromosome 6q32 of the rat (http://ratmap.ims.u-tokyo.ac.jp). However in humans this gene has been found on chromosome 6q21-6q22.2 and not on 14q32. If this were to be the case, it would mean that 6q32 of the rat may be homologous to the human chromosome 14q32, as well as 6q21-6q22.2. For the purposes of finding suitable candidates, it was vital to find out whether Macs really did map onto the rat chromosome 6q32. The sequences between BB/OK, KWR and BB.KWR (chr. 6; BB.6W) were compared based on polymorphism in the Macs gene (between 901 and 1321, KWR has 1 amino acid more) between BB/OK and wild animals (KWR). All BB.6W animals displayed the genotype of the BB/OK rat. This meant that the possibility of Macs being located on chromosome 6q32 of the rat was excluded and that a mapping error had arisen. This further meant that candidate searches by homology comparisons could be limited to 12q13 in mice and 14q32 in humans.

Since there is also mapping in the diabetes protective region of the gene Akt1/Pkb, this being essential for β-cell function (34, 35), initially attempts were made to position Akt1/Pkb relatively accurately in the region. Because BB/OK and SHR are polymorphous (intron between 1321 and 1561), mapping could be carried out using the subcongenic BB.6S lines Akt1/PKB between Ighe and Ckb, in other words outside the diabetes protective region. This therefore excluded this gene as a possible candidate.

As a result of the parallel in vivo characterisation of the BB.6S rats in comparison to the parent strain of BB/OK, further findings were obtained as to the function of the possible candidate gene:

1. Morphological examinations of the pancreas of normoglycaemic BB/OK and BB.6S rats displayed significant differences. At the age of 30 days significantly more islets of Langerhans were infiltrated with lymphocytes (insulitis) for BB/6S than for BB/OK. On every 12 BB.6S or BB/OK examined, 51.2±4.6% and 7.5±2.5% (p<0.001) respectively of the islets were infiltrated. At the age of 90 days, the percentage of islets with insulitis was comparable between the two strains, being at around 50% (37). Based on this, insulitis occurred both in BB/OK and in BB.6S, leading to destruction of the beta cells and resulting in type 1 diabetes in around 86% of BB/OK animals and only about 14% of BB.6S. This would indicate immune tolerance induction in BB.6S.

There were significant differences also between fresh manifestations of diabetic BB/OK and BB.6S as regards insulin content and percentage of insulin positive β-cells. Compared to diabetic BB/OK, the insulin content (0.15±0.03 as against 0.42±0.13 pmol/mg, p<0.05) and the percentage of insulin positive β-cells (0.07±0.02 as against 0.19±0.06%, p<0.05) was significantly lower than for diabetic BB.6S, which might indicate less aggressive destruction of insulin producing β-cells than in the case of BB/OK rats (37).

2. Studies on fracture healing in BB/OK and diabetes resistant LEW.1A rats (38) showed that BB/OK rat bone was clearly more brittle than that of LEW.1 A rats, indicating possible disruption to BB/OK calcium metabolism. Various studies on calcium metabolism only resulted in significant differences in the serum calcetonin content between BB/OK and BB.6S rats. Significantly higher calciton readings were demonstrated for BB/OK as against BB.6S (2.4±1.57 as against 1.0±0.65 pmol/ml; p=0.0002). Given that calcitonin not only restricts calcium release in bone, but can also inhibit insulin secretion via various proteins and receptors (39, 40), an attempt was made to obtain further indication of the interplay of calcium (Ca) and manifestation of diabetes by modulating the calcium supply through feeding of BB/Ok and BB.6S.

BB/OK and BB.6S rats were fed a standard diet (0.8% Ca), a high Ca (2%) and a low Ca diet (0,4%) and observed for development of diabetes during their first 30 weeks of life. Whereas in the case of BB/OK rats diet had no demonstrable influence on incidence of disease (control=88%; low Ca=92%; high Ca=90%), in the case of BB.6S rats, significantly more animals fell ill with diabetes once fed a high Ca diet (control=12%; low Ca=18%; high Ca=45%, p=0.02). In addition, the high Ca diet led to similarities in body mass and serum cholesterol content for BB/OK and BB.6S. There were no significant differences between the lines within the first 30 weeks of life. Additional in vivo manipulations, such as administration of Ca channel blockers or various Ca antagonists, did not result in any in vivo effects.

3. In vitro examinations of isolated islets of Langerhans of BB/OK and BB.6S rats showed that variation in Ca concentration in the culture medium did not lead to any difference between the two lines either in insulin content or glucose stimulated insulin secretion.

There were however significant differences in protein expression using Western blot analysis. The calcium-binding protein, calbindin-D28k, was already twice as high in BB.6S rat islets under basal conditions as that of BB/OK rats. Increasing calcium concentration in the medium clearly increased the protein expression of calbindin-D28k in BB.6S islets, whereas expression in BB/OK rats was almost uninfluenced by Ca. Calbindin-D28k is a cytosolic calcium-binding protein, which is primarily expressed in the kidney, but also in the pancreas and brain, and which can prevent apoptotic (gene controlled) cell death (41, 42). In functional terms it is supposed that calbindin-D28k is involved in regulation of calcium reabsorption.

Given that the candidate gene

1. can almost switch off the two essential diabetogenic genes, Iddm1 and Iddm2,

2. is involved in immune tolerance and apoptotic cell death,

3. as well as in regulation of calcium, calcium-binding proteins (calbindin-D28k etc.) and hormones (calcitonin, etc.),

4. and in dyslipidemia,

the conclusion was drawn that the gene is a multifunctional transcription factor.

The Candidate Gene

After homology comparison and gene function tests, the favourite as candidate gene for the present invention was the transcription factor Yin Yang 1 (YY1), which in all probability is mapped in the diabetes protective region in both humans and mice. This gene can both activate and repress transcription of a large number of cellular and viral genes, or indeed initiate transcription on its own. As a consequence it amounts to a multifunctional transcription factor.

YY1 belongs to the class of regulatory transcription factors (RTF), in other words binding is sequence-specific to proximal and distal DNA regulation elements. In addition, it has the capacity of influencing the transcription mechanism directly or indirectly. Due to its multi-facetted molecular structure (activation domain, repression domain, zinc finger structures) it is possible for it to implement differential gene activity. This means that it can function as a temporal, spatial, cell-specific or signal-transducing parameter. Its function modules carry out the tasks of specific DNA recognition, transcription activation, and transcription repression, as well as interaction with other proteins.

Gene Structure—a Comparison

Yin Yang protein is characterised by the local build-up of aspartic acid (D) and glutamic acid (E) between the AA regions 43 to 53. This segment is homologous between rats and humans, but the number and position of E and D differ from each other. Rats have 5×E and 6×D, whereas humans have 6×E and 5×D. In rats a histidine cluster is added from 54 and 79 and in humans from 54 to 82. Rats are 3 histidines short as compared to the sequence in humans. In humans there then follows a GA/GK rich domain from 154 to 198. In rats this positional region is shifted by 3 AA positions towards the N-terminal end. (151-195). The four zinc fingers of the human sequence begin at position 298 and end at position 407. In the rat sequence this region runs from 295 to 404. Total AA length in humans is 414 and in rats AA length is 411 (see Sequence Protocol: SEQ ID NO:2 and 4; the designation “SEQ ID NO:” corresponds here and in what follows to the sequence code “<400>” according to WIPO Standard ST.25).

Functional Domains

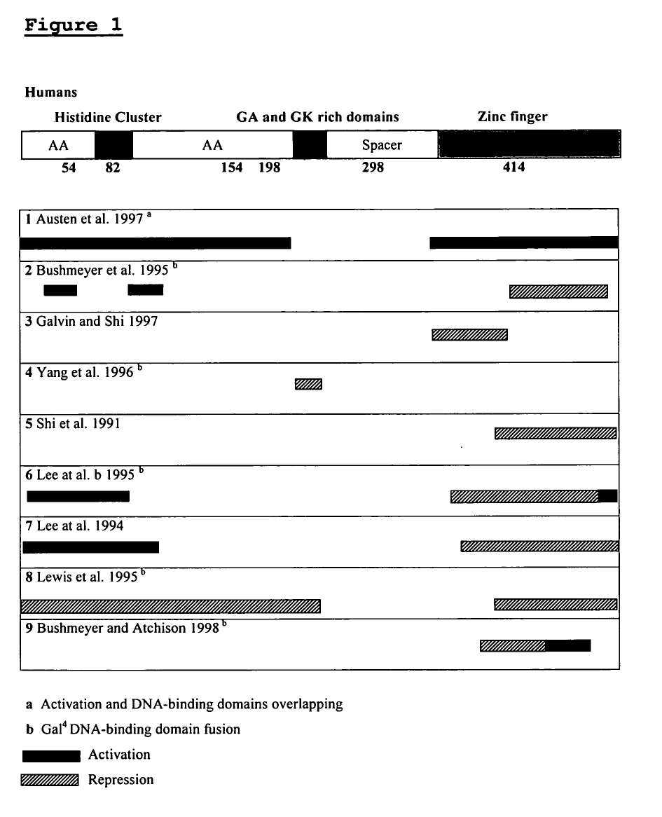

Many research groups have analysed the structure and function of YY1 using deletion mutants, as well as reporter constructs. These results are summarised in FIG. 1.

Two activation domains were found, confirming the above. These are located in the region of the N-terminal end and of the zinc finger. In addition, further domains of two groups with activating function could be located in the region of the C-terminus (397-414 and 370-397) (43, 44). The argument was put forward that the N-terminal activation domain was being masked by the C-terminal domain, meaning that if the C-terminal domain were deleted the N-terminal domain would be revealed, with the result that YY1 may act as a constitutive agitator. These results led to the conclusion that YY1 becomes an activator only under certain conditions. It turned out that E1A was able to change Yin Yang into an activator (43).

Repression domains could be located for the zinc finger region. Here repression activity was attributed to zinc finger 1 and 2 with zinc finger 2 and 3 being held to be responsible for DNA binding (45). Observing, however, the X-ray co-crystal structure of zinc finger 2 and 3, it was found that this was able to attack both the DNA bases and the DNA phosphate backbone (46). For zinc finger 1, contact points could only be indicated for the phosphate backbone, and for zinc finger 4 only for a few bases.

Further repression domains turned out to be overlapping with the activation domain at positions 1 to 201, as well as between 170 and 200 (47, 48).

Interpretation of these results by Thomas et al. (49) showed overall that there are only two repression domains in the YY1 gene, in the region from 170 to 200 and in the zinc finger region, that may perhaps be able to interact together (cf. FIG. 1).

Protein/Protein Interactions

Yin Yang is able to interact with proteins, such as transcription factors and coactivators/corepressors. Two domains that interact are the regions around 150 to 170 (central domain), where the GA/GK rich domain with spacer fragments, the C-terminal region, and the zinc fingers with parts of the spacer are all located.

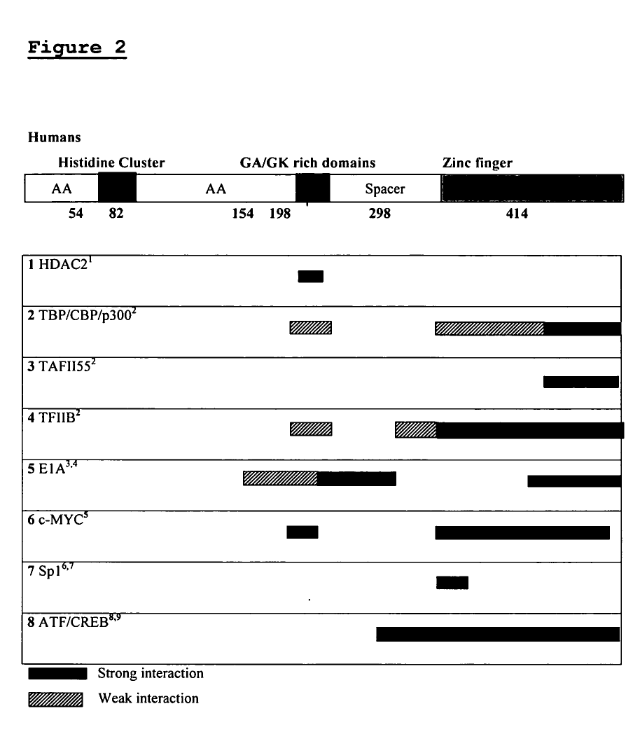

Some proteins, such as TBP, CBP/p300, TFIIB, E1A and c-MYC can bind to both regions (43, 45, 47, 50, 51). Proteins that can only interact with one of these two domains are: HDAC2 (central domain), SP1 and ATF/CREB (C-terminal domain) (45, 48, 52-54) (cf. FIG. 2).

Galvin and Shi have shown (45) that repression of CREB and SP1 by YY1 is activator-specific. The authors suggested that YY1 interferes with the target(s) of the activators, instead of directly binding with these two factors.

Only one protein, E1A, has so far been found that directly interacts with the activation domain of YY1 (43, 47, 55). Since this discovery there has been discussion as to whether YY1 can change its function as repressor into one of activator, possibly by masking its repression domains by modification or, on the other hand, by exposing its activation domain.

Promotor Binding

Characterised by four Cys2-His2 zinc fingers, Yin Yang is able either to activate or inhibit transcription, but this is dependent on the promotor sequence of the genes and the concentration of YY. The consensus sequence (C/t/a)CATN(T/a) (T/g/c), which is present in many promoters of viral and cellular genes, can be bound via the zinc finger (56).

Repression Models

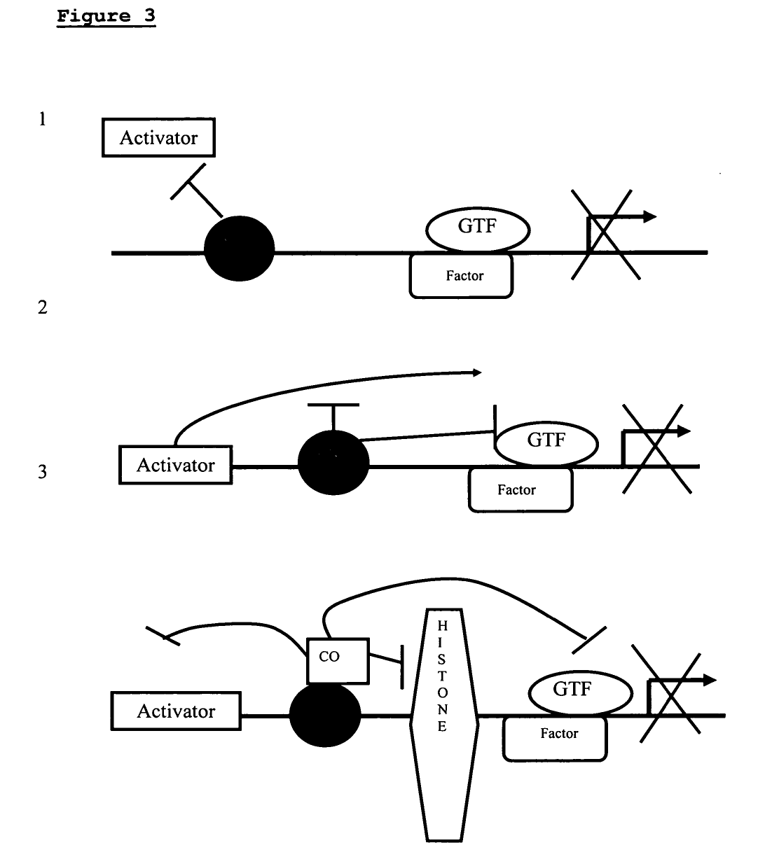

Along with controlled transcription activation, repression of the same is an important control mechanism of gene activity. Repression by reversal or neutralisation of gene-activating processes can be effected by YY1. Several repression regions were found in the YY1 gene segment (see FIG. 3).

Three models of repressor activity are known to current science. In the first model YY1 displaces the activator. In the second model it exhibits disruptive characteristics. It impedes the activator and general transcription factors (GTF) in the carrying out of their function.

Indirect repression (the third model) is exemplified by YY1 in the recruitment of corepressors. It has an indirect influence on chromatin structure (49). Of particular interest here are HDAC 1, 2 and 3, as corepressors. HDACs are linked to a loosening of chromatin structure. All three proteins are global regulators of RPD3, in other words they are able to deacetylate histones (in vitro). In addition, the HDACs can directly block transcription in controlling the promotor (48, 57-60). In the case of overexpression of HDAC 2 the group of Yang et al. (48) was able to show that the repression function of YY1 is amplified. Given the discovery that HDAC 2 operates in the same complex as HDAC 1, the same must be true of HDAC 1.

Activation Models

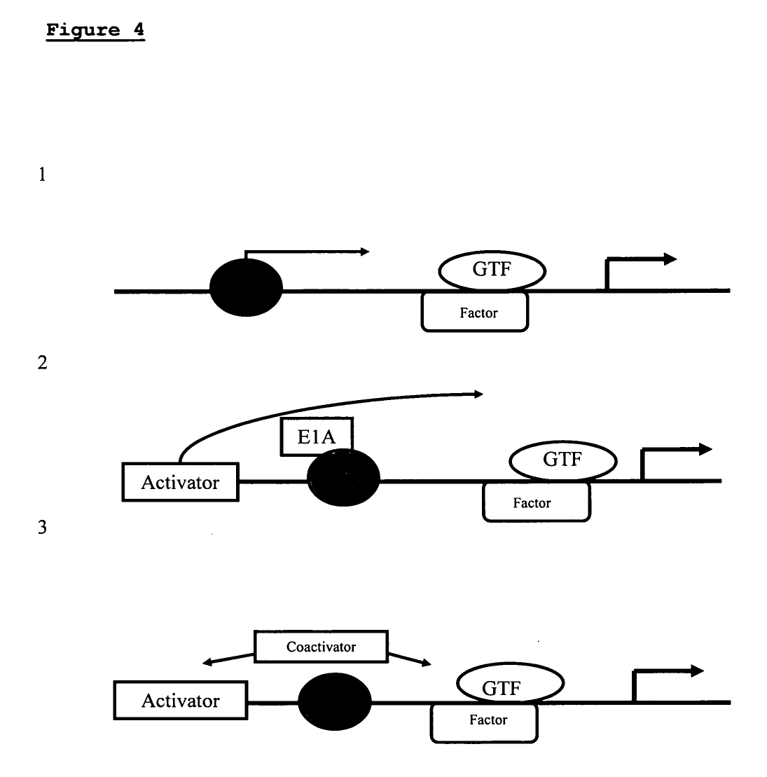

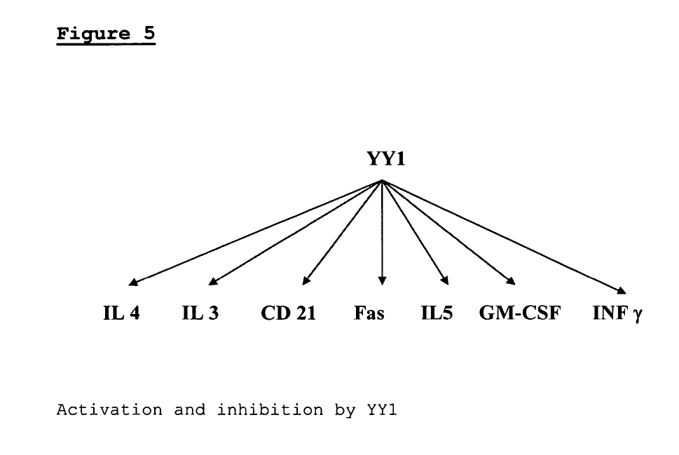

Three models are known for transcription activation by Yin Yang(see FIG. 4). Direct activation has been able to be demonstrated using the adenovirus E1A protein by the group Shi et al (61). Transcription stimulation is obtained by direct YY1 interaction with the GTFs, TAFII55, TBP and TFIIB (62, 63). The second mechanism could be explained by means of the AAV P5 initiator element (64). In addition there is discussion as to whether YY1 undergoes structural change when it interacts with other proteins. In the third model, Yin Yang recruits coactivators that interact with other transcription factors. This model possibly also includes modification of chromatin by p300. As a result of a loosening of chromatin structure by means of p300 (HAT activity), access to DNA would be made easier and efficient binding would be possible (51, 55, 65).

Regulation of YY1 by Acetylation and Deacetylation:

Regulation of the transcription factor at the post-translational level is via interaction with other proteins.

YY1 transcription activator activity is directly dependent on its association with the coactivators p300, CBP, and PCAF. These coactivators contain histone acetyltransferase (HAT) and are therefore capable of transferring acetyl groups. In contrast, YY1 repression activity is associated with histone deacetylase 2 (HDAC2) in the region 170-200 of the repression domain.

Selective association of YY1 with HAT or HDAC is decisive as to whether it functions as activator or repressor.

Acetylation and Deacetylation via p300,PCAF and DHAC:

Yin Yang has 2 acetylation domains. The first is in the region between 170 and 200, and the second overlaps the zinc finger region at the C-terminal end between 261-414.

Acetylations in the region 170 to 200 are implemented by p300 and PCAF on the lysine residues. Six pairs of lysines are in the first domain, with only 3 different lysines being acetylated by p300 and PCAF. To obtain maximal repression activity, YY1 must be acetylated at all three points. If only one lysine were to be replaced by one arginine, there would be no more maximal repression activity, given the conformation change that would occur.

One further decisive point concerning acetylation between 170 and 200 is that the probability of DHAC binding is significantly increased and, as a result, deacetylation by HDAC 1 and 2 can also occur in this region.

Surprisingly, it is also possible for HDAC to bind in the second acetylation region (261-333). In contrast to the first acetylation domain, however, no deacetylation occurs here.

In addition, it could be shown that YY1 DNA binding activity is reduced by acetylation of PCAF in the overlapping region of the zinc finger (261-333) (66).

Fine Mapping of YY1

Given that until December 2002 the rat sequence was not as yet in the gene bank, in accordance with the present invention the mouse gene sequence was used to recruit primers, since the first task was to fine map YY1. It had to be determined beyond doubt that the gene really was in the diabetes protective region.

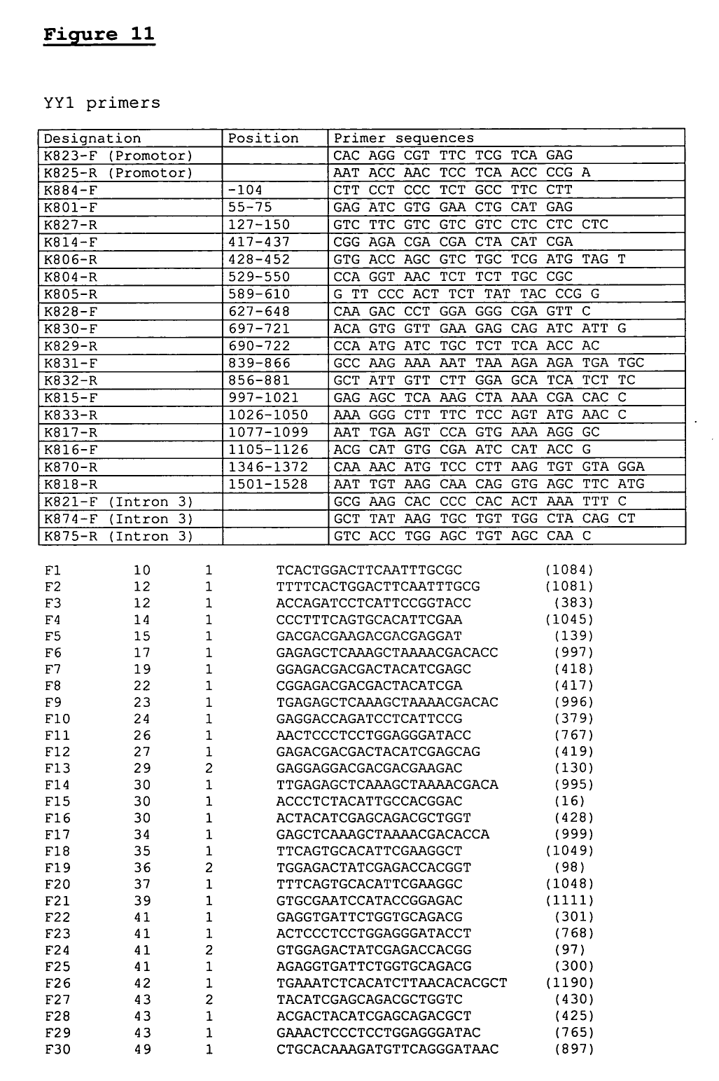

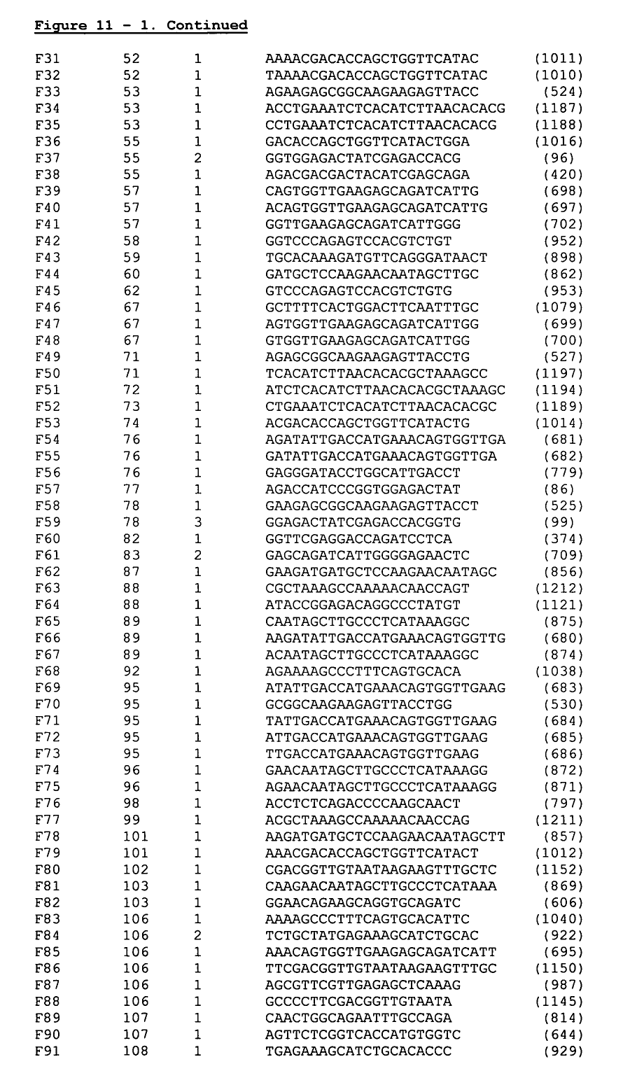

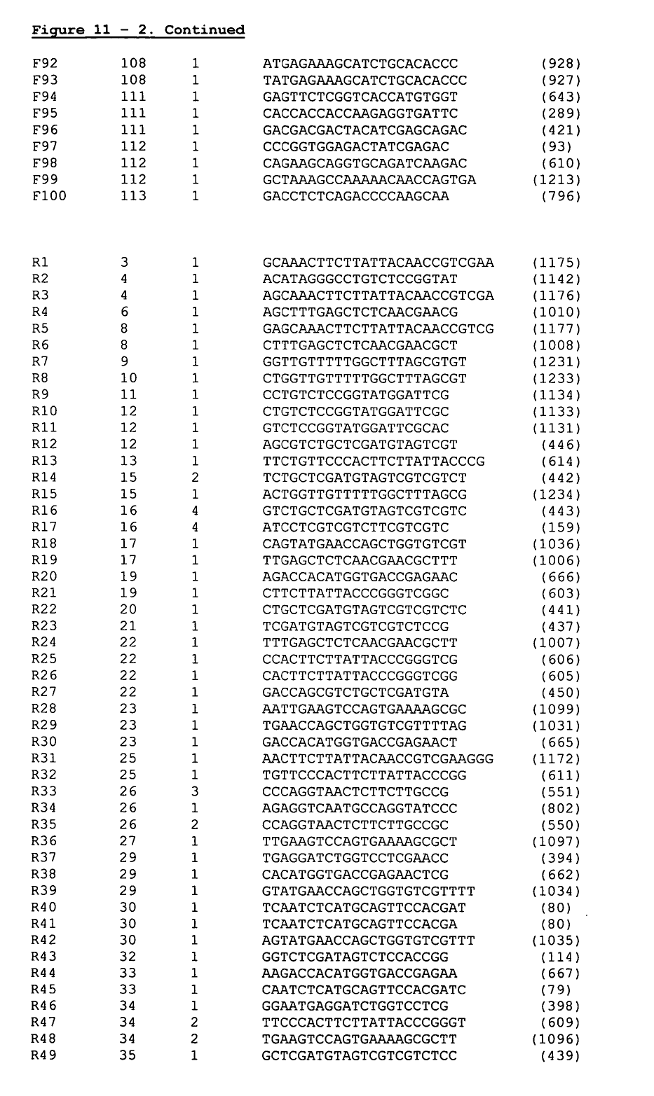

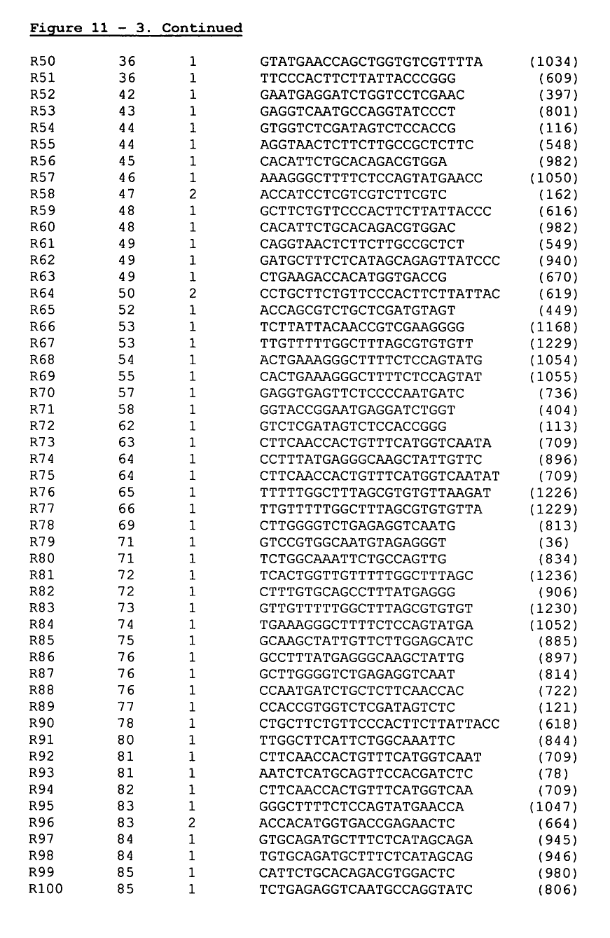

Given that the chance of finding polymorphism in the intron is very high, primers for amplification of the intron were used (cf. FIG. 11; intron 1: K828-F/K829-R; intron 2: K830-F/K832-R; intron 3: K831-F/K833-R; intron 4: K815-F/K817-R; K815-F/K875; K821-F/K817-R; K821-F/K870-R; K874-F/K870-R). Only one gene product that could be sequenced with genomic DNA was obtained from intron 4. Intron has 633 base pairs (bp) and differs between BB and SHR rats at positions 323 (t-a), 502 (g-c) and 528 (a-c). This polymorphism was used for exact positioning of YY1 on chromosome 6q32 together with the subcongenic BB.6S lines. YY1 was mapped between D6Mgh2 and Ighe and is therefore in the diabetes protective region.

To check whether the polymorphism in intron 4 also occurs in other strains or whether other differences might occur, intron 4 was sequenced and compared for the following rats: 2 wild animals (wild type, M+F), 1 animal of each of diabetes resistant, inbred DA/K, BN/Mol, LEW/K and WOKW rats.

WOKW rats do not develop hyperglycaemia (=diabetes), but a full metabolic syndrome (obesity, hyperinsulinaemia, dyslipidemia, impaired glucose tolerance, hypertonia) and, like BB/OK rats, come from the same Wistar rat crossbred population of BioBreeding Laboratories, Ottawa, Canada (67, 68).

It turned out that the intron sequence between BB/OK and wild animals, as well as between SHR and DA, BN, LEW.1W, and also WOKW, is identical.

YY1 Sequencing

Both genomic and cDNA were so amplified using PCR and various primers that overlapping gene products for sequencing became available (cf. FIG. 11; promotor: K823-F/K825-R*; promotor and exon 1: K884-F/K806-R; exon 1: K801-F/K804-R; K814-F/K832-R*; exon 2 and 3: K828-F/K833-R; K831-F/K817-R; exon 4: K815-F/K870-R; K815-F/K818-R; K816-F/K819-R; K834-F/K836-R). The PCR and sequencing were carried out as described in the Material & Methods section (M&M).

The YY1 rat gene contains 1236 base pairs (bp) and consists of 411 amino acids (AA), as confirmed by Nishiyama et al. 2003 (69). Sequence differences between BB/OK and SHR and/or BB.6S were demonstrated at position 603 (c-t), 980 (t-g) and 1004 (g-a) (cf. SEQ ID NO:1 and 3). This base exchange leads to a change in the amino acids at position 303 and 311 in the zinc finger region (cf. SEQ ID NO:2 and 4). On BB/OK rats the AAs methionine (nucleotide 907-910 from startcodon) and arginine (nucleotide 931-933 from startcodon), and on SHR rats the AAs arginine and lysine are encoded.

The promotor region was only partly sequenced from the transcription start (−72), with one more region of 470 bp with the GC box remaining. No differences between BB/OK and SHR could be demonstrated.

After sequence comparison with the mouse the promotor region should be about 891 bp from the transcription start (Acc: L13969, 1-464, 86% homology). In a comparison with the human sequence (Acc: AF047455 promotor in this sequence only −42bp) correlations between −636 to −585 (216-269; 47/54; 87% homology) and −464 to −392 (392-464; 64/73; 87%) bases from the transcription start were to be found.

Nucleic Acid and Protein Sequence Comparison Between Rats and Humans

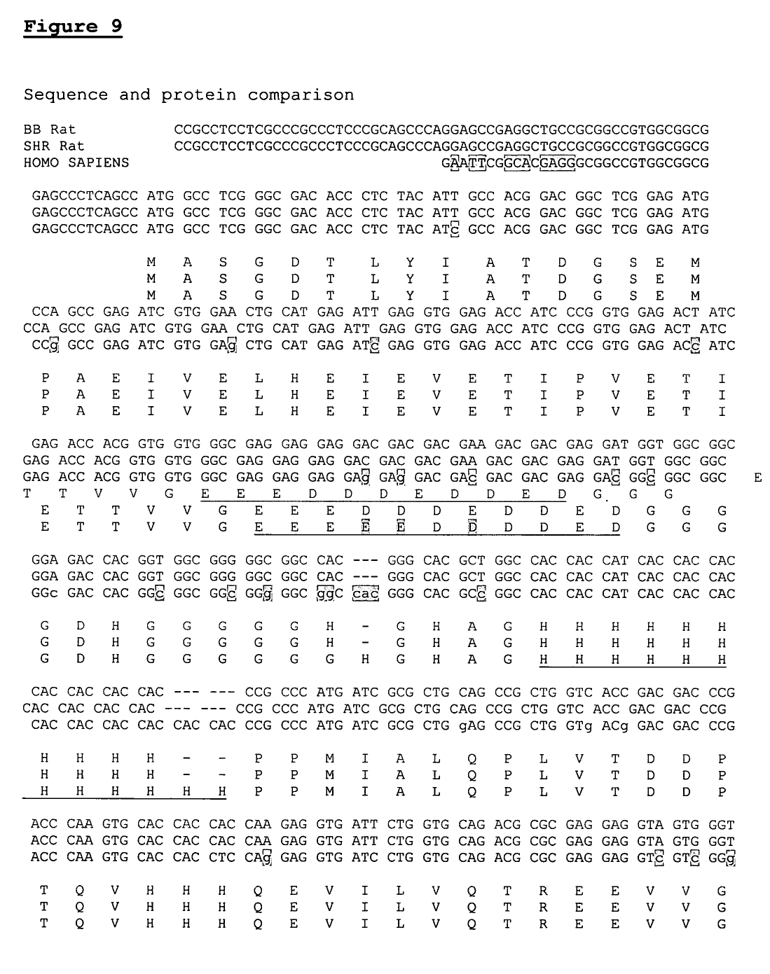

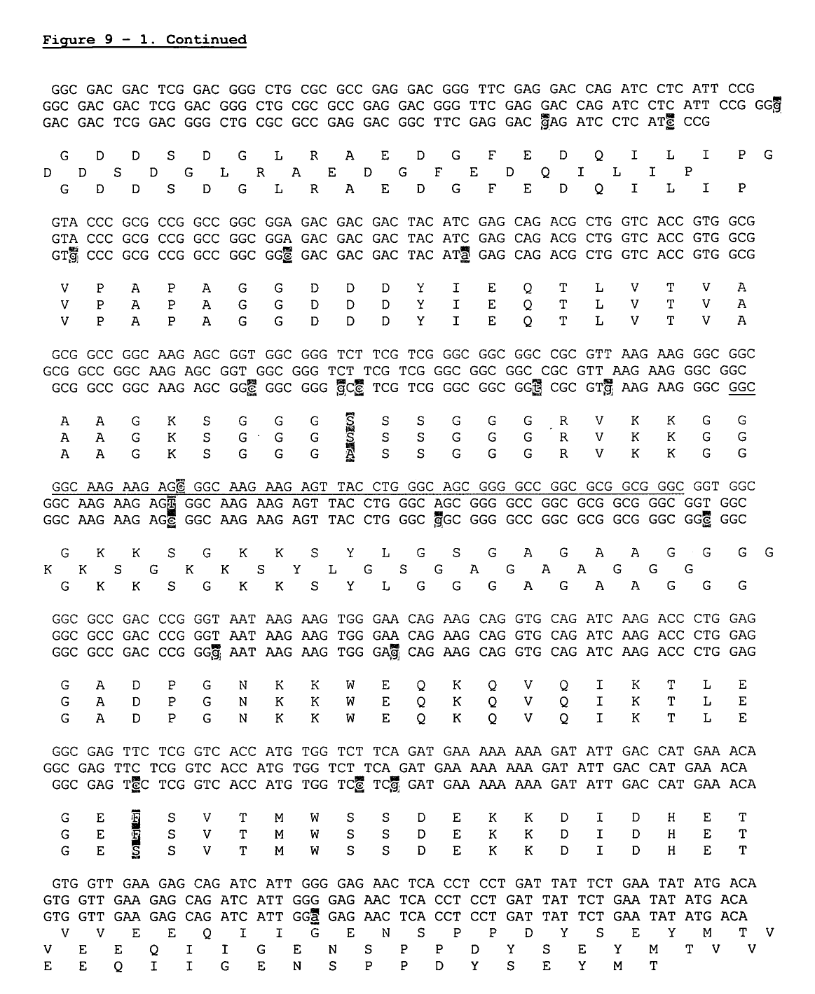

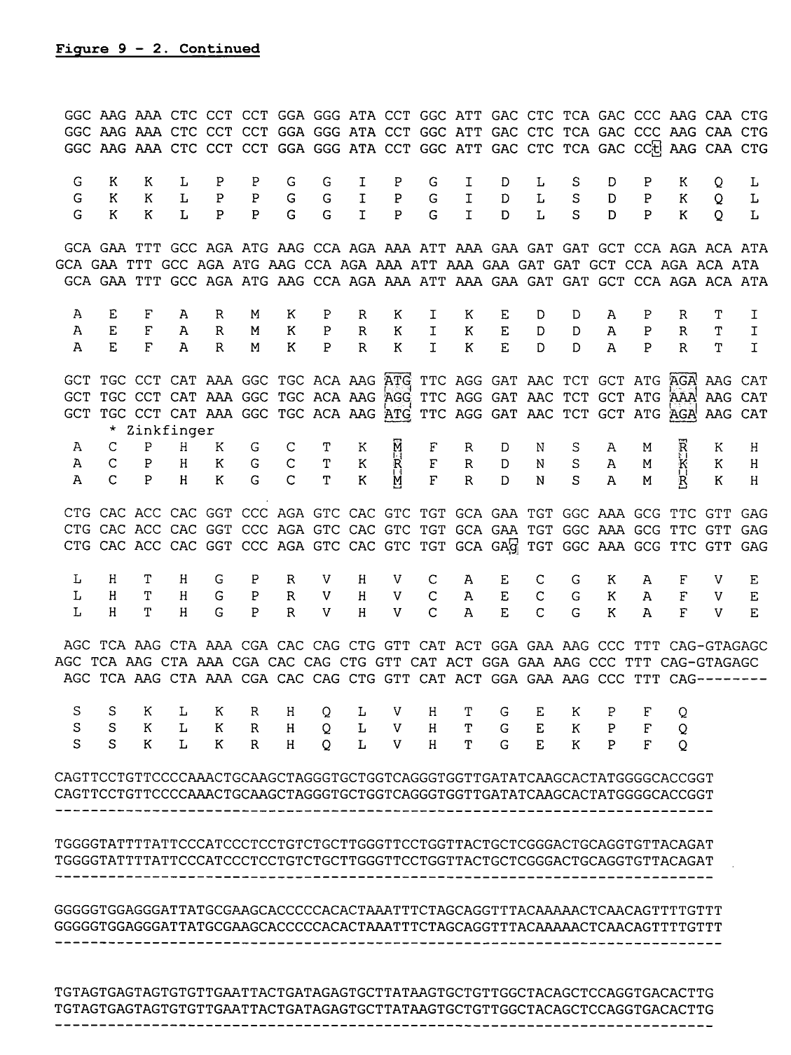

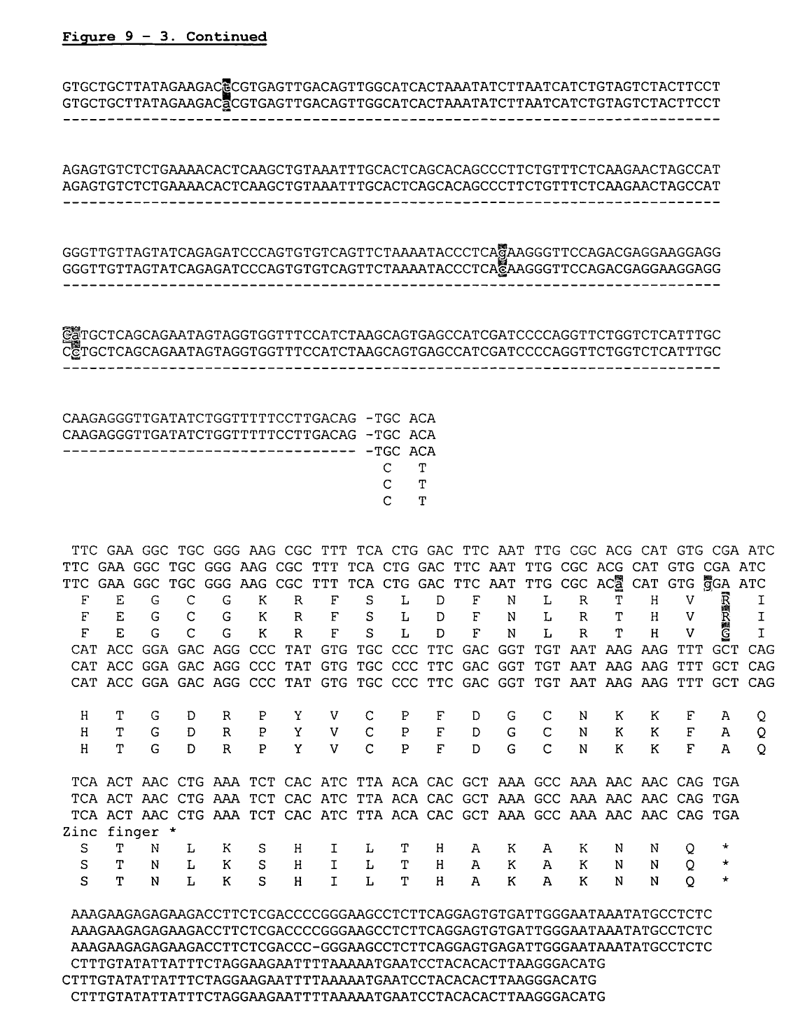

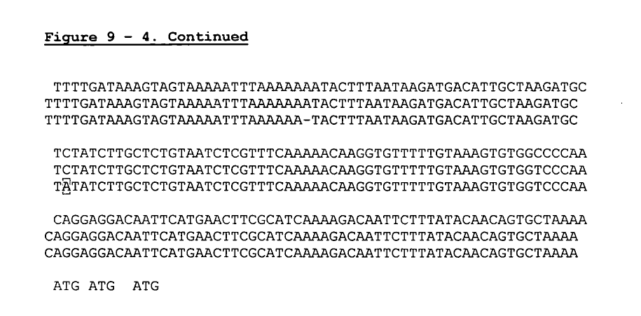

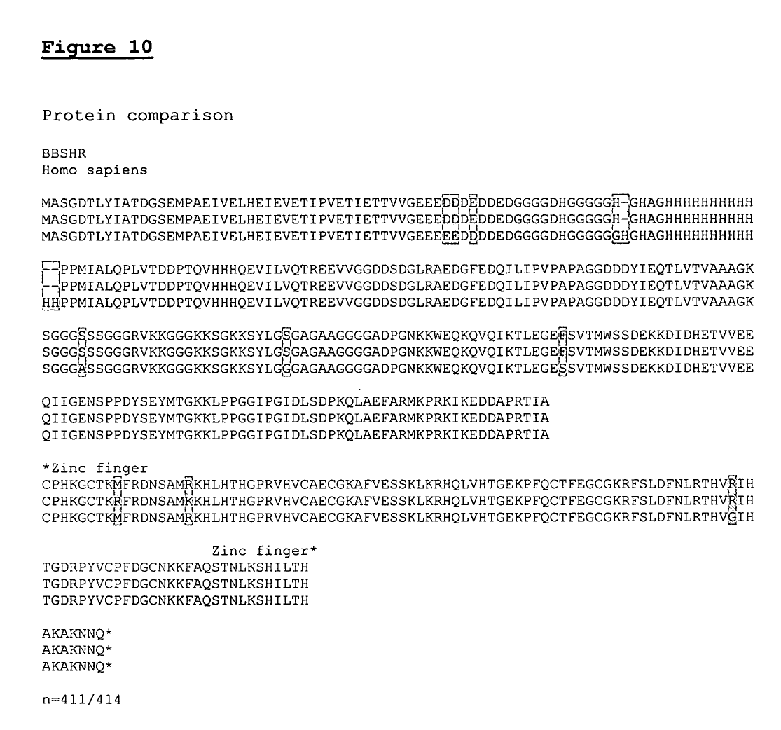

Sequence comparison between BB/OK, SHR rats and humans, as summarised in FIGS. 9 and 10, indicated bp homology of 95.6% and 95.3% respectively and AAs of 96.9 and 96.4% respectively. If we take into account the fact that the rats have 3 AAs missing (3 histidines−1×between activation and first repression domain, position 66 in humans, and 2 in the first repression domain—instead of 12 histidines the rat only has 10), homology between rats and humans in the coding region increases to 97.0 (1206/1239) and 96.8% (1200/1239) for the bps and 97.6% (401/411) and 97.1% (399/411) for the AAs. In the zinc finger that includes 330 bps and 110 AAs, 99% of the bps (327/330) and AAs (109/110)are homologous for the BB/OK rat, and for SHR rats 98% of the bps (325/330) and 97.3% (107/110) of the AAs are homologous.

Gene Expression Studies

To check the extent to which the differences in sequence affect YY1 gene expression, gene expression studies were undertaken.

mRNA was extracted from isolated islets of Langerhans, the pancreas, liver, brain, thymus and spleen of 8-day-old rats of the inbred strains BB/OK, SHR, BB.6S and LEW (“neutral” control), transcribed into ssDNA and used in the gene expression studies (cf. Material & Methods section).

It was repeatedly shown that the YY1 zinc finger region, using the primers K831-F/K818-R and K831-F/K870-R, is strongly expressed (YY1 zinc finger/GPDH intensity=0.19±0.04) in isolated islets of Langerhans of the BB/OK rat and LEW rat respectively, and significantly reduced in expression in those of the SHR and BB.6S rats (YY1 zinc finger/GPDH intensity=0.03±0.02; p<0.001). In the pancreas similar difference in expression was evident, but in a somewhat attenuated form. Investigation of expression using the primers K831-F and K870-R indicated that in the pancreas, liver and brain of BB.6S rats, a second, approximately 150 bps shorter band occurred, which could not be observed either in SHR or LEW. Also of interest was the fact that sexual differences in gene expression occurred. Expression in male animals was always stronger than in females. YY1 is expressed in all other organs examined. Striking differences between the strains, except between males and females, were not observed.

Sequencing of the Second Band for BB.6S

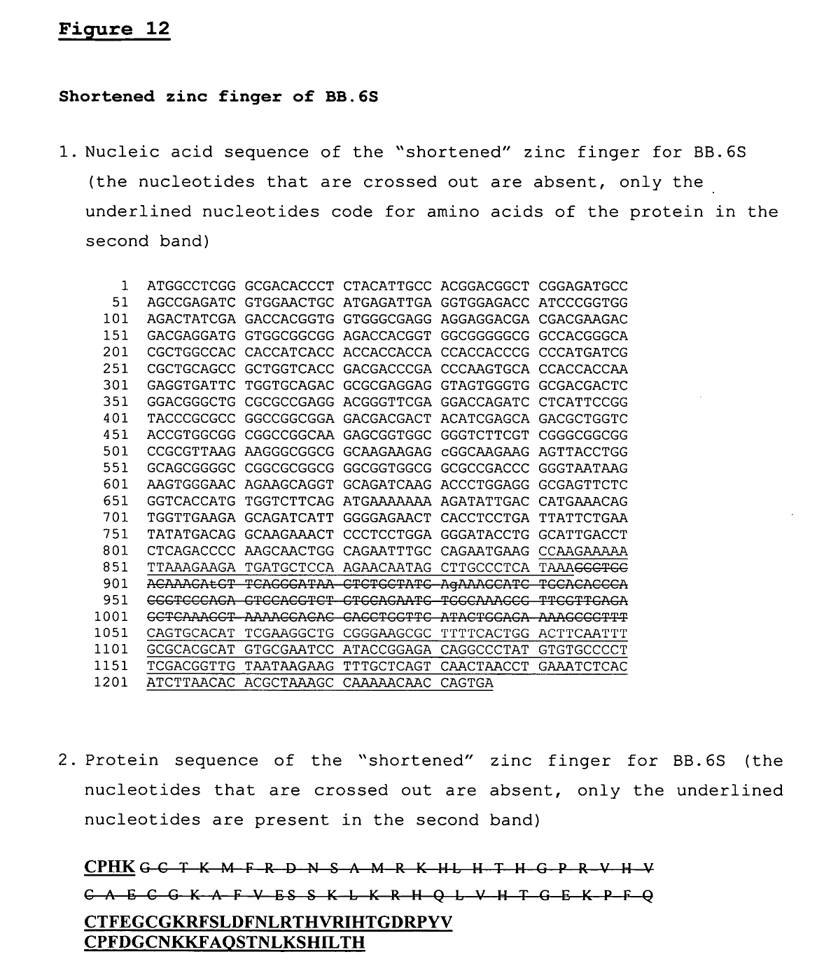

Given that a second band only occurred in BB.6S rats in the pancreas, liver and brain, this second band was eluted, amplified and sequenced (cf. M&M). Results showed that this band is a truncated zinc finger sequence (cf. FIG. 12 and SEQ ID NO:7). Based on this, a part of zinc finger 1 is missing, and zinc finger 2 is completely absent (967 to 1125; cf. SEQ ID NO:8).

### CONCLUSIONS

Given that it could be shown that

1. YY1 maps in the diabetes protective region as a multifunctional transcription factor,

2. Differences in sequence can be demonstrated, with a change in AA between BB/OK and SHR in the zinc finger region (AA at position 303—-methionine/arginine and 311 arginine/lysine are different) and in intron 4 in the zinc finger,

3. On BB/OK rats YY1 is clearly expressed in isolated islets of Langerhans and the pancreas, in the case of SHR and BB.6S rats scarcely at all, whereas in the other organs investigated, expression was to a comparable degree, but with 2 bands for BB.6S after amplification of the zinc finger region,

it was concluded that YY1 for BB.6S is underexpressed in the islets of Langerhans and therefore acts in a diabetes protective manner, with the second band in the pancreas and other tissues also being of importance for diabetes protection, especially since a special role is attributed to zinc finger 2 in transcription. An observation which is caused by the AA change in the zinc finger region and/or the intron (differing splice characteristics for BB/OK and BB.6S).

Given that the change in the zinc finger region has a diabetes protective effect, regulation/activity of several genes is impaired/altered. That the intron has a role in diabetes protection is supported by that fact that differences in sequence between BB/OK and SHR could be demonstrated, but not between BB/OK and wild animals. The sequence compared between BB/OK and wild animals was the same. This also explains why BB.6S but not BB.6W rats are protected from developing diabetes.

As a consequence of the findings made in accordance with the present invention, modulation of the multifunctional transcription factor YY1 at the level of expression and regulation on the one hand for the first time opens up a route to preventing type 1 diabetes, and on the other allows autoimmune diseases per se to be prevented by means of this modulation. In addition, other illnesses such as cancer, AIDS, lipometabolic disorders and hypertonia can be positively influenced.

The subject of the present invention is therefore a protein that displays the amino acid sequence illustrated in SEQ ID NO:4. Also included are homologues of the protein, which display arginine and, at position 311, lysine. The degree of homology is at least 95%, preferably at least 97%, and most preferably 99%.

The invention also relates to peptides that are fragments of one of the proteins mentioned and display an amino acid sequence that contains the sequence region that comprises the amino acid positions 303 and 311 in SEQ ID NO:4 (or 306 and 314 in SEQ ID NO:6). The length of the peptides in accordance with the present invention is, for example, 53 to 315 amino acids, preferably for example 315, 117 or 53 amino acids, where the peptides preferably comprise the sequence regions from position 1 to 315, 295 to 411 and 299 to 351, respectively.

According to the present invention, moreover, the following fragments are important: amino acids 165-214; 255-333; 255-411. Numbering is based on the numbering of SEQ ID NO:4.

In accordance with a preferred embodiment of the invention, also of importance are peptides that only cover a region from, for instance, position 295-310 or 305-320 (numbering based on SEQ ID NO:4) and therefore include one of the two mutated amino acids.

The invention also relates to a nucleic acid that encodes a previously mentioned protein or peptide. The nucleic acid encoding protein having SEQ ID NO:4 preferably displays the nucleic acid sequence represented in SEQ ID NO:3. A nucleic acid coding for the aforementioned homologues displays a nucleic acid sequence that contains the sequence region comprising nucleic acid positions 979-981 and 1003-1005 in SEQ ID NO:3. The codons at the aforementioned positions may of course vary as a result of degeneration of the genetic code, provided they encode the amino acids arginine and lysine, as the case may be.

In accordance with the present invention, the following intron sequence is moreover important: 1126-1758. (The position of the exons in SEQ ID NO:3 is: exon 1: 73 to 729; exon 2: 730 to 825; exon 3: 826 to 978; exon 4: 979 to 1125; exon 5: 1759 to 1938; see Sequence Protocol.) Numbering is based on the numbering of SEQ ID NO:3.

Diabetes Mellitus Type 1 and Autoimmune Diseases per se

As already mentioned, type 1 diabetes results from the selective destruction of insulin producing β-cells in the pancreas. The demise of the β-cells is controlled by auto-reactive T-cells that are directed against β-cell specific antigens (autoantigens). Insulitis is linked with the manifestation of diabetes (69). This process is characterised by lymphocytic infiltration of the β-cell. Given that insulitis does not always have to be the response to β-cell destruction, a distinction is made between benign and destructive insulitis (70). This process correlates with cytokine production, as could be shown for animal models (BB rats) and for humans (71-78). The cytokines IFNγ and IFNα play a major role during destructive insulitis. IL10 and TGFβ are seen as major factors for benign insulitis. In addition, insulitis could be induced in non-diabetic mice by TNFα, TNFβ, as well as IL6, but this did not lead to β-cell destruction. In the case of transgenic expression of IFNα, IFNγ and IL2, it could be shown that non-diabetic mice develop insulitis and autoimmune type 1 diabetes. Based on this, IFNγ, IFNα, IL2 and IL10 are involved in the development of type 1 diabetes and TNFα, IL4, IL6, and TGFβ in its prevention (71-81).

Given that all BB.6S rats develop insulitis, but that diabetes only occurs in about 15% of animals (82), YY1 must also play a role in the insulitis process. Either YY1 triggers the protective cytokines, suppresses the pathogenic cytokines, or else balances both cytokine groups in such a way that on the one hand β-cell destructive insulitis occurs, or else, on the other, benign insulitis that does not lead to autoimmune diabetes comes to bear. Since it is still in a position to activate IL4 (83) and inhibit IFNγ (84), in accordance with the present invention it is assumed that it also shifts the relation of the T-helper cell populations, TH1 and TH2, to TH2's advantage.

If in type 1 diabetics the AA sequence of YY1 is changed via mutation, it can no longer bind to certain sequences in the DNA, or else its affinity to the binding site is reduced. In other words, it can no longer bind to the IFNγ promotor, or else its binding affinity is too low, nor can it continue to inhibit IFNγ synthesis. Overproduction of IFNγ would ensue. Such overproduction could then lead to insulitis. Because IFNγ activates the TH1 response by upregulation of the numbers of MHC class I proteins on the γ-cell surface (85, 86), thereby influencing T-cell differentiation in the direction of TH1 leading to a reduction in the TH2 response, there would be a shift between TH1 and TH2, as postulated in autoimmune diabetes. It is moreover known that macrophages are initially detectable in the β-cell. Because IFNγ is a stimulator for macrophages, early immigration into the γ-cell can be explained by the fact that IFNγ is no longer inhibited via YY1. In addition YY1 is also predestined to induce IL4 synthesis (83). If this also does not take place because of sequence change or underproduction of YY1, the TH1 response can no longer be suppressed by IL4 as protective cytokine. Based on the increased activity of TH1, cytotoxic T-cells (CTL or CD8⁺) and NK cells take on their function in the β-cell as “killer cells”. Apoptotic β-cell death, induced by FAS receptors during interaction with the FAS ligands, can still be additionally induced by YY1(87). If the transcription factor is mutated in cases of type 1 diabetics, it can no longer fully inhibit FAS expression. This means that FAS appears on the surface of β-cells, and the apoptotic signal cascade is activated. The result is β-cell death and type 1 diabetes as a consequence.

Since neonatal thymectomy on animal models (BB rats and NOD mice) prevents the development of type 1 diabetes (88), it is also supposed amongst other things that in the case of type 1 diabetes autoreactive T-cells are already developing during T-cell differentiation. In other words, YY1 would already have to intervene in the early stages of T-cell development, perhaps on the following lines.

To develop a functional lymphocyte repertoire the precursor cells, so-called prethymocytes, must pass through a wide variety of stages of development in the thymus. Since only about 2% of mature T-cells can leave the thymus, there is strict death or survival selection during T-cell maturing. One point of regulation is anchored in the early thymocyte stage, the “double negative” (DN). Here the pre-T-cell receptor (preTCR) on the surface, consisting of CD44 (β-chain) and the invariant pTα chain, decides whether entry into the cell cycle is possible. This control point is also called the “beta checkpoint”. The immature T-cells remain in this stage until the right α-chain gene is rearranged and the T-cell receptor (TCR), consisting of an α- and β-chain, can be expressed on the surface. If both T-cell receptors CD4 plus CD8 appear together with CD3 on the surface, these T-cells are now termed “double positive” (DP). Around 95% do not reach this stage. This underlines the importance of the control point in the transition between the DN and the DP stages (89).

Regulation of the expression of the pTα-chain is seen here as the critical factor. pTα-gene transcription appears up until now to be decisively regulated, particularly by the upstream region, the enhancer. Several proteins, amongst them also YY1, which are able to bind to this region, have been identified (90). Further binding proteins of the enhancer are SP1/3, ZBP-89 and c-MYC. If we look at these proteins in connection with YY1, we can deduce that all, with the exception of ZBP-89, interact with YY1, meaning that T-cell maturing is also seen as a further regulation point of YY1. This supposition is lent support by the findings of Iritani et. al. (91). They were able to show that c-MYC alone is unable to regulate pTα-expression. Only highly different c-MYC concentrations seemed to have some partial influence on cell growth arrest in the later DP stage. If YY1 is overexpressed in the thymus, it triggers pTα transcription. Vice-versa, in the case of underexpression, it hinders pTα transcription. In over and underexpression, as well as sequence changes of YY1, T-cells can be arrested during their development or else transferred into another T-cell stage.

Because T-cell maturing and its selection in all autoimmune diseases, such as rheumatoid arthritis, multiple sclerosis, autoimmune encephalomyelopathy, celiac disease etc., is decisive, alongside type 1 diabetes autoimmune diseases per se can be influenced by YY1, and this is obtained by upregulation or downregulation of YY1.

It is assumed that the YY1 sequence is different between type 1 diabetics and healthy probands. Several sequence variants are expected for type 1 diabetics, given that along with classical type 1 diabetics there are other forms of type 1 diabetes, such as LADA (Latent Autoimmune Diabetes in Adults) for instance. These sequence variants are to be used as genetic markers for prediabetics. Proof of the change is based on DNA sequencing, where whole blood is obtained, DNA and/or RNA, which is transcribed to ssDNA, is isolated and then sequenced.

Parallel to this, the expression profile for type 1 diabetics is drawn up. Given the fact that the illness of any given type 1 diabetic progresses in an individual way, it is expected that the expression profile differs from proband to proband. Depending on the age of the proband, and on gender, autoantibody status [GAD (glutamate decarboxylase), IA-2 (protein tyrosine phosphatase), ICA (cytoplasmic islet cell autoantibodies)] of the BMI, as well as the HLA genotype (HLA-DQB1), various expression profiles are expected. Based on this, the relevant prophylaxis/therapy must also be individually adapted. Due to genetic heterogeneity, YY1 interacts with a different genetic background in the case of each individual, so that YY1 expression between the individuals is subject to great variation.

Downregulation can be achieved by applying antisense and production of specific antibodies for the probands in question. The antisense and antibody structure results from the sequence order of the proband. The amount of antisense as well as antibodies to be applied results from the expression profile of the proband (upregulation and downregulation cf. M&M).

That YY1 itself acts as an autoantigen stands to reason, since the trigger antigen for destruction of insulin producing beta cells is still not known to date. If there is confirmation that YY1 functions as an autoantigen then tolerance may be induced via oral administration of YY1, as is already the case using hypersensibilisation of patients having allergies. This involves identifying the sequence variants for each proband or proband group and then producing the mutated form synthetically, so as to be able to administer it orally. This should allow a shift in the TH1/TH2 profile to be obtained so as to counteract autoimmune destruction.

The possibility of reducing development of type 1 diabetes in BB/OK rats by application of naked cDNA or DNA has been shown in the latest results of a pilot study. At 2, 8, 12, 16, 20, 25 and 30 days 174 offspring of non-diabetic mothers were applied the following: Naked DNA of the primers K815 and K817, K831 and K818 (FIG. 11), as well as purified PCR products using the primers K815 and K817 (amplification of genomic BB/OK DNA, YY1 intron, sequence 1069 to 1804), and K831 and K870 (amplification of BB/OK cDNA, exon 3, 4 and 5, zinc finger, sequence 911 to 1125 and 1758 to 2078) depending on dose (100, 200 and 400 ng in 50 μl) once on the above days, subcutaneously. In addition non-diabetic BB/OK females were systematically mated and then treated on sperm testing and on the 4^{th}, 8^{th}, 12^{th}, 16^{th} and 18^{th} day of pregnancy with 100 or 400 ng of naked DNA (primer K815/K817). The 24 offspring of these mothers and the 174 postpartuum treated offspring were observed for signs of diabetes until their 30^{th} week. Untreated as well as treated BB/OK rats served as the control group, these being applied 50 μl water subcutaneously at the same age. After 30 weeks 93% of the treated (13/14) and 86% of the untreated control animals (19/22) had fallen ill. Very considerable reduction in diabetes frequency was observed in 2 trial groups. Frequency decreased to 62% (8/13) under application of the zinc finger PCR product (400 ng) and to 50% (6/12) in the case of offspring that were born to the mothers treated with 400 ng. All other trial groups fell ill with an incidence comparable to that of the control groups (74 to 100%), with some dependency on dosage being observed. A further study showed that application of 400 ng/50 μl PCR product, amplified with the primers K831/K870, could further reduce the incidence of diabetes. Compared to the “water control” (16/19=84%) significantly fewer BB/OK rats fell ill (16/19 as against 13/27; p=0.013) in the treated group. In view of these initial results it is assumed that more frequent and/or longer application of naked DNA and/or PCR products can lead to reduction in diabetes incidence in BB/OK rats to around 0%.

Protooncogenes and Cancer

The c-MYC gene was originally discovered in the 70s as the cellular homologue of the retroviral v-myc oncogene. Embryonal lethality results from deletion of this gene. c-MYC protein is the product of a c-MYC protooncogene which—just as YY1—is a multifunctional transcription factor. c-MYC plays a critical role in differentiation, cell growth, proliferation, transformation and apoptosis of cells. Dysregulations of c-MYC expression are connected to abnormal malignant cell growth and tumour formation in lung cancer, breast cancer and colo-rectal cancer. This shows that c-MYC, as a strong regulator of cell growth, cell differentiation and cell proliferation, must be both well controlled and balanced so as to prevent the development of carcinomas (102-104).

Given that, in the absence of the essential partner protein (MAX), YY1, as a versatile regulator, can bind directly to the C-terminal part of the basic helix-loop-helix (bHLH) zinc finger of c-MYC, YY1 also plays an important role in the development of cancer. Here YY1 is able to use each of its four zinc fingers to bind to the c-MYC protein. It is not however possible for YY1 to interrupt binding between the c-MYC/MAX complex. Given, however, that sequence-specific DNA binding of YY1 is not blocked, in spite of complex formation between YY1 and c-MYC, regulation at the DNA level by YY1 is still possible.

The human c-MYC gene codes two polypeptides (439 and 453 amino acids) with molecular weight of 64 and 67 kDa and is located in the cell nucleus. Translation start of the larger protein is at the end of the first exon, whereas the smaller and generally prevalent form of the protein is initiated by translation in the second exon.

C-MYC is a transcription factor that displays basic helix-loop-helix/leucine zipper domains (bHLH-LZ), where these conserved domains are a part of many transcription factors and mediate protein/protein, as well as protein/DNA interactions.

C-MYC belongs to a family of genes that also includes other members such as a N-, L-, S- and B-myc (105-108).

Binding of c-MYC to the DNA is via the so-called E-box, a specific regulatory sequence of the 5′-CACGTG-3′ bases. For transcription activation the c-MYC protein must form heterodimer complexes with the partner protein MAX (109). MAX is also one of the bHLH-LZ proteins, but it does not have a transactivating domain like c-MYC (110). Heterodimerization with MAX is essential for c-MYC functions, such as regulation of the cell cycle, induction of apoptosis or transformation of cells. MAX also forms complexes with the bHLH-LZ proteins of the MAD family. These heterodimers also bind specifically to E-boxes and have a transcription repressive effect via Sin3 mediated recruitment of histone deacetylases (111).

Based on this, downregulation or upregulation of YY1, dependent on its many various interactions with other genes, and via c-MYC also, ought to make it possible to influence the formation of cancers, and this is highlighted in the following observations.

BB/OK rats do not just develop type 1 diabetes, but also tumours, as a long-term study has shown. 202 BB/OK rats were observed until their death from natural causes. 87 of the 202 developed diabetes. The remaining 115 animals survived on average 576±79 days. Only 37% (42/115) lived longer than 400 days. The majority of the animals (73/115) died between their 200^{th} and 400^{th} day, primarily as a result of tumours (50/115). The organs concerned were the liver, lymph nodes, lung, bowel, pancreas and spleen (112). An analogous study was carried out with BB.6S rats. 47 animals were observed to their 600^{th} day of life. 6 out of 47 rats (12.8%) became diabetic by their 30^{th} week, and 2 more animals showed signs of the illness at 437 and 560 days. 62% of the animals (24 out of 39 non-diabetic animals) survived the observation period of 600 days. 6 animals died without macroscopic identification of the cause of death. 9 animals had to be sacrificed because of their poor general condition, with cause of death being diagnosed in 7 cases as obstruction of the bowel, and in 2 animals due to tumors in the liver. Based on this, the congenic BB.6S rats live significantly longer than BB/OK rats and the tumour development rate is significantly lower in BB.6S than in BB/OK rats (50/115 as against 2/39, p<0.0001). In view of these findings, the exchanged region, and therefore YY1 in the case of BB.6S rats, seems to result in considerable reduction in tumour formation and increase in life expectancy. And based on this, downregulation of YY1 should be able to prevent tumour formation and increase life expectancy.

Lipid Metabolism

Steroid Hormone Synthesis

Steroids are synthesised in specialised cells of the adrenal glands, ovules, testes, placenta and in the brain. They are essential for maintaining normal homeostasis throughout the body. Synthesis of all steroid hormones begins with the conversion of cholesterol into pregnenolon. This is the first enzymatic step which takes place at the matrix of the inner mitochondrial membrane.

Steroidogenic acute regulatory protein (StAR) plays a key role in cholesterol transport from the outer to the inner mitochondrial membrane. Cholesterol transport is what limits the rate of steroidogenesis. Mutations in the StAR gene cause potentially lethal conditions that are known as congenital lipoid adrenal hyperplasia. The same observations have been made on StAR knockout mice (131).

StAR expression can be regulated positively and negatively by agencies that predominantly affect the promotor. Hormone-stimulated steroid synthesis is accompanied by a sharp rise in the StAR mRNA level. cAMP has a positive and rapid effect on the rise in StAR mRNA, but does not seem to affect the promotor sequence directly.

The first transcription factor as a potential regulator of the StAR gene was steroidogenic factor 1 (SF 1), also called orphan nuclear receptor transcription factor.

The StAR promotor has various consensus binding sequences for SF 1. Two of these at position −97 and −42 are highly conserved. Another one at position −132 has only been found in mice and rats.

Another candidate is the CCAAT/enhancer binding protein (C/EBPs), that belongs to the family of b region/leucine zipper transcription factors. Two members of this family are expressed in steroidogenic cells (C/EMPα and C/EMPβ). The StAR promotor has two possible binding sites for C/EMP. SF 1 and C/EMP form a complex on the StAR promotor.

Both human and rat StAR promotor has, moreover, binding sites for SREBP-1a (sterol regulatory element binding protein-1a). SREBP-1a is an important activator for the StAR promotor.

Other transcription factors such as SF 1, NF-Y, YY1, and SP1 are involved in the effect of SREBP on the StAR promotor. SREBP-1a regulates the StAR promotor in a coordinating manner along with the other factors.

CREB can also bind and quickly activate the transcription (cAMP binding).

DAX-1 binds directly to the hairpin structure of the StAR promotor, as well as directly to SF-1 and inhibits expression in each case (131).

If SREBP-1a has a coordinating role in the production of cholesterol in steroid hormone synthesis, it then becomes of critical importance in cholesterol homeostasis. The StAR promotor in rats has 5 so-called SRE binding sites (sterol regulatory element), to which the activated form of SREBP-1a can bind and activate transcription (132). A further SRE binding site has been found in the human StAR promotor to which both SREBP-1a and YY1 can bind and which under certain conditions is active with high concentrations of SREBP-1a (133).

SREBPs belong to the family of basic helix-loop-helix leucine zipper (bHLH-Zip) transcription factors, which are synthesised as 125 kDa membrane-bound precursor proteins in the endoplasmatic reticulum and, if there is a lack of sterol in the cell as a result of enzymatic cleavage of the 68 kDa N-terminal region of the protein containing the bHLH-Zip domain are activated and migrate into the nucleus. There they bind specifically to the DNA sequence of SRE (sterol regulatory element) and activate transcription of their target genes (134, 135).

Hitherto three SREBPs have been described, called SREBP-1a, SREBP-1c and SREBP-2. SREBP-1a and SREBP-1c are expressed by the same gene using alternative promoters. SREBP-2 is expressed by a separate gene (135, 136).

Cholestol genes, which have classical SRE binding sites in their promoters, are strongly and effectively activated by SREBP-1a and SREBP-2. SREBP-1c is inactive for these binding sites.

SREBP-1c, also called ADD 1, has one special feature. It binds to E-boxes (universal cis-elements for bHLH proteins). This means that it displays dual binding specificity to both classical palindromic E-boxes and non-palindromic SREs. This unique binding specificity is due to the tyrosine residue in the basic region, which is unique for the SREBP family, since all known bHLH proteins have arginine at this position (136). This exchange destroys the transactivity of all SREBPs for SRE binding sites, but markedly increases activity of SREBP-1 for E-box binding (see SREBP-1c), as well as that of SREBP-2. This latter is however inactive and does not activate the target gene (136).

The SREBPs are transcription factors of differing strength and generally need cofactors. NF-Y, SP1, and CBP (CREB binding protein) are such cofactors.

SREBP target genes are split into 2 groups (136):

Cholesterol biosynthesis genes
HMG-CoA synthase and reductase
Farnesyl diphosphate synthase
Squalene synthase
SREBP-2
LDL receptor
HDL receptor (137)

All the above contain the classical SRE sequence (ATCACCCCAC) or the SRE 3 motif (CTCACACGAG) and adjoining cofactor binding sites for NF-Y or SP1 in their promoters.

Lipogenic enzyme genes are nutritionally regulated at the transcription level (e.g. glucose and insulin)
Acetyl-CoA carboxylase
Fatty acid synthase (FAS, increases TG)
Stearyl-CoA desaturase 1 and 2
Glycerol-3-phosphate acyltransferase
Diazepam-binding inhibitor (Acyl-CoA binding protein)
Spot 14 (liver enzyme S 14)

The SREBP binding and activation sites in the promotors of these genes appear to differ from the classical SRE consensus sequences and are termed SRE-like sequences.

A few other enzymes such as liver-type pyruvate kinase (PK) and glucokinase (GK) contain E-box or E-box like sequences in the promotor, and this can mean that they are sensitive to carbohydrates, glucose and insulin, making them potential SREBP targets.

All actively growing cell cultures predominantly produced SREBP-1a and SREBP-2, whereas most organs, including the livers of adult animals, produce SREBP-1c and SREBP-2. All SREBPs are able to activate each of the known target genes, but with varying efficiency. SREBP-1c is weaker (shorter transactivation domain) than SREBP-1a and SREBP-2.

The specific role of the SREBP isoforms in vivo was examined in transgenic mice. Different overexpression showed that the SREBP-1 isoforms selectively activate the fatty acid synthesis genes and that SERBP-2 specifically controls cholesterol biosynthesis.

SREBP-1a and SREBP-1c play a large role in nutritionally dependent induction of hepatic lipogenic enzymes and cholesterogenesis. SREBP-2, on the other hand, determines sterol regulation by degradation of membrane-bound protein so as to prepare the active form for migration into the nucleus. SREBP-1 controls lipogenic enzymes via self-regulation of its own transcription level.

The fact that the SREBPs are relatively weak transcription factors, and that they require cofactors, has already been mentioned. Bennett et. al. (138), for instance, showed that activation of the SREBPs by sterol deficiency in vivo results in enhanced SP1 binding to a sequence adjacent to the SREBP binding site in the promotor for the LDL receptor gene. Similarly the two coregulating factors NF-Y and CREB (cAMP responsive element binding protein) are more strongly bound to the HMG-CoA reductase promotor. SREBP activation increases histone acetylation of H 3, but not of H 4, in the chromatin of both promoters (HMG-CoA synthase and reductase). The results show that slight differences in the pattern of nucleus histone acetylation play a role in selective gene activation. This shows that binding of the transcription factors to the DNA is a necessary but not a sufficient condition of transactivation (138).

SREBP-1 can activate some other E-box or E-Box like sequences in the FAS promotor and in the S14 promotor, but it is completely inactive for degenerated E-box sequences in the PK promotor for example. Promotors with SRE binding sites must have NF-Y or SP1 as cofactors for SREBP activation. SREBP-1 has considerably greater binding affinity and is more efficient in activating promotors containing SRE than promoters containing E-box.

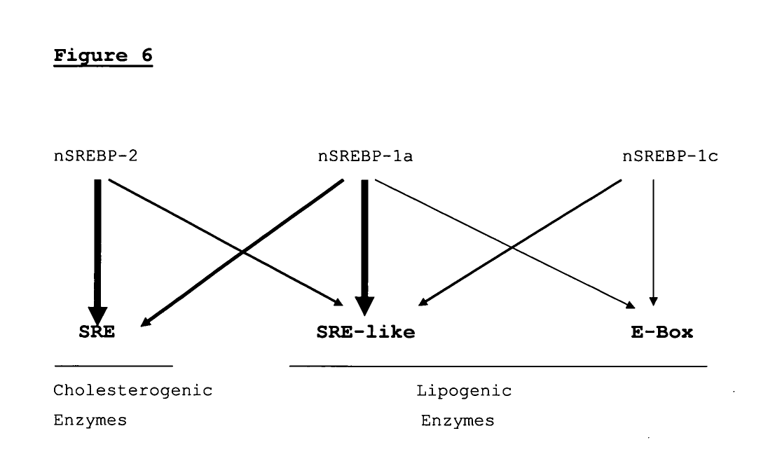

In lipogenic enzymes, which contain promotors with SRE-like sequences, all isoforms of SREBP activate transcription (SREBP-1a more strongly than SREBP-1c, SREBP-1a preferred to SREBP-2). Binding of SREBP isomers to the various gene promoters (136) is illustrated in FIG. 6.

Influence of YY1 on Regulation of the Lipogenic Genes

YY1 is known as a transcription modulator that can act both as enhancer and repressor, but also as initiator binding protein, this being dependent on the origin of the cell and the binding sites of the promotor. YY1 is also able to activate and inhibit the same promotor, if the intracellular milieu is changed, the binding element is mutated (changed) in the promotor, or the DNA sequence surrounding the binding site is altered.

With regard to the genes under consideration, the following YY1 influences may be deduced.

YY1 has high affinity to SREBP-1a. In the StAR promotor there is a YY1 binding site that overlaps with the proximal binding site for SREBP-1a. Simultaneous binding of SREBP-1a and YY1 to the StAR promotor represses the latter's transcription. This fact further influences cholesterol transport and therefore steroid hormone synthesis.

YY1 acts as an activator of specific repressors, competing with the binding sites of the SREBPs and their cofactors, or sterically hindering the binding of the SREBPs, for example to SREs of the lipogenic genes. It acts both as a type I repressor (cofactor binding, complex formation) and as a type II repressor (direct binding to the DNA, steric blocking of binding sites). By mutation of the binding site in YY1 it could be shown that SREBP-1a activation of the StAR promotor increased many times. Investigations of transfected HepG2 cells showed that YY1 mutants (YY Δ 296-331, YY Δ 399-414, YY Δ 334-414 or YY Δ 154-199) did not repress sterol-activated expression of the LDL receptor and FPPS receptor, for instance. The mutations YY Δ 334-414 or YY Δ 154-199 prevent nuclear localisation, DNA binding and interaction with CBP. YY Δ 296-331 and YY Δ 399-414 were inactive in repression of SREBP-1a dependent induction of expression of the HMG-CoA synthase promotor (134).

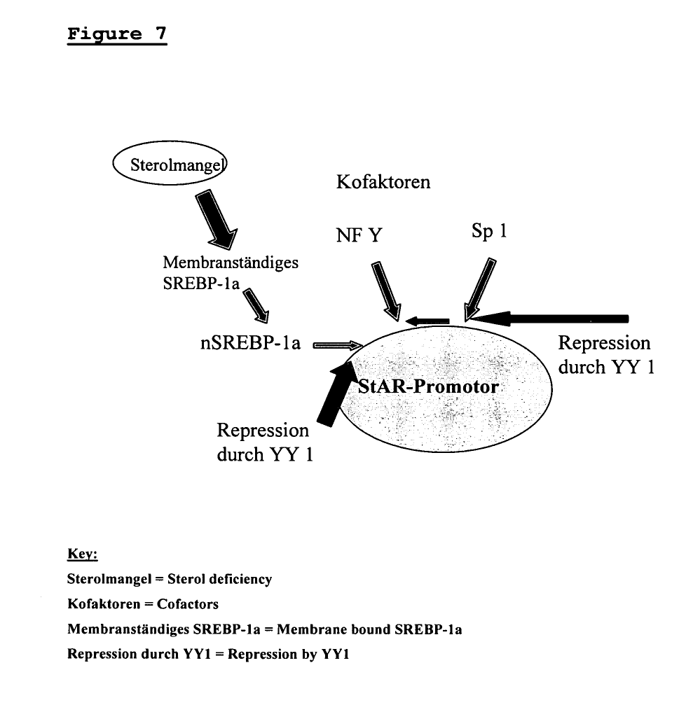

Numerous other promotors of the sterol regulated genes contain potential binding sites for YY1 (CCAT or ACAT) that are overlapping or adjecent to binding sites of the coactivators and/or the transcription activators. These can be displaced or blocked by YY1 and gene expression repressed as a result (cf. FIG. 7).

Based on the same principle, the other promoters are repressed by YY1.

HDL receptor→positively regulated by SREBP-1a plus SP1 [symbol] YY1 (repr.)

LDL receptor Fatty acid synthase→positively regulated by SREBP-1a plus SP1 [symbol] YY1 (repr.)

Farnesyl synthase HGM-Co A synthase→positively regulated by SREBP-1a plus NF-Y [symbol] YY1 (repr.)

HGM-CoA reductase gene expression is not repressed by YY1.

Binding synergism of SREBP and NF-Y or SP1 to promotors of the SREBP responsive genes, including SREBP-2, leads to transcription activation, which controls cholesterol homeostasis (LDL receptor, HDL receptor, HMG-CoA synthase and reductase, FPP (farnesyl phosphate) synthase, squalene synthase), fatty acid synthase (fatty acid synthase and acetyl-CoA acetylase), fatty acid degradation (stearyl-CoA desaturase) and triglyceride synthesis (glycerol-3-phosphate acetyltransferase) (134).

YY1 can intervene in these processes via the mechanisms mentioned above. YY1 can also hinder recognition of the domains on SREBP-1a for RNA polymerase II by interaction with SREBP-1a, and thereby repress transcription, and this is another important regulatory mechanism of transcription regulation (134).

Influencing SREBP Activation in Various Illnesses

SREBP-1c is predominantly expressed in hepatic cells, and it is stimulated in these cells also by glucose and insulin both at mRNA and protein level (139).

In experimental streptozotocin diabetes of rats massive increase in expression of SREBP-1a and fatty acid synthase was found. This resulted in heavy triglyceride accumulation (TG). Treatment with insulin was able to reduce both. In kidney cells with high glucose, SREBP-1a and SREBP-1c mRNA and protein synthesis were enhanced, as well as fatty acid synthesis and therefore TG accumulation. SREBP-1 expression is enhanced in diabetes mellitus. SREBP-1 plays an important role in the enhancement of lipid synthesis, TG accumulation, mesangial expansion, glomerulosclerosis and proteinuria, in that it enhances expression of TNFβ and vascular endothelial growth factor (140).

In cases of type 2 diabetics a significant reduction in SREBP-1 expression in subcutaneous fatty tissue and skeletal muscle was found. Ex vivo this effect could be eliminated using TNFα (141).

For this reason lipid metabolism can be influenced accordingly by upregulation or downregulation of YY1, as the following results corroborate.

As already mentioned at the start, BB/OK rats and BB.6S rats do not just differ significantly in the incidence of diabetes and the age of manifestation. At the age of 12 weeks BB.6S rats are also heavier in comparison to BB/OK rats, and serum total cholesterol is significantly increased in males and females, as well as serum triglyceride in females (24). After their 24^{th} week, there is also a significant increase in serum triglyceride in the male BB.6S rats compared to the BB/OK rats. Based on these findings it appeared right to conclude that YY1 is also involved in fat metabolism. For this reason a pilot study was carried out to test the extent to which application of YY1 antisense of the 4 zinc fingers can influence lipids. 600 ng/100 μl antisense were applied to 10-12 male rats of the BB/OK, LEW.1A, LEW.1W, WOKW and SHR strains for 2 weeks, starting in their 9^{th} week. The serum triglyceride (TG), serum total cholesterol (Chol) and serum HDL cholesterol (HDL) were determined before (8^{th} week) and after application (10^{th} week). As expected, the genetically and phenotypically different strains reacted differently, as summarised below.

| | | | | HDL/Chol |
|---|---|---|---|---|
| Strain | TG | Chol | HDL | ratio# |
| BB/OK | ↑* | | | ↑** |
| BB.LL | ↑* | | | |
| LEW.1A | | ↓** | ↓* | ↑* |
| LEW.1W | | ↓** | ↓** | |
| SHR | ↓* | ↓** | ↓** | ↑** |
| WOKW | | ↓** | ↓** | |

↑ ↓ significantly increased or lowered.
*p < 0.05
**p < 0.01
#HDL/cholesterol ratio

Interestingly, of the 12 BB/OK rats only 7 fell ill (58%) by their 30^{th} week, and this once again underlines the diabetes protective effect of YY1 PCR products (see p. 31/32).

As described for type 1 diabetes, it should be possible to influence the blood fats as well, depending on the expression profile of the probands (cf. Material & Methods section).

Vitamin D, Calcium Metabolism and Development Processes

Along with parathormone (PTH), calcitonin (CT) and PTH related peptides (PTHrP), vitamin D is also a main regulator of calcium metabolism. From 1966 it has been known that vitamin D must be present in an active form if it is to fulfil its functional tasks (143). The active form results from hydroxylation at the C25 and the C1 atom. This form is termed 1.25-dihydroxycholecalciferol or vitamin D₃. One of the functions of calciferol is to counteract any reduction in the plasma calcium level. This is achieved via increased intestinal calcium resorption, by increased renal calcium resorption and increased calcium mobilisation from the skeletal system (144). Intestinal resorption needs an active transport system (calcium-binding proteins, calbindin) that is only found if vitamin D₃ is present. In addition vitamin D₃ is also responsible for ensuring the intestinal mucous cells remain capable of functioning. Calbindins, osteocalcin, matrix Gla protein, osteopontin, collagen type I etc., are all induced by calcerifol. Calciferol also influences proliferation, differentiation, hormone secretion and apoptosis. The importance of vitamin D₃ becomes clear in deficiency symptoms, so-called hypovitaminoses (rachitis, osteomalacia) (144).

Once the vitamin D receptors (VDR; 145, 146) were discovered, the mechanism of action could be investigated. Using the VDR it could be shown that vitamin D is located in the nucleus of keratinocytes of the skin, and islets of Langerhans of the pancreas, lymphocytes, promyelocytes etc. The active form of vitamin D is lipophil, meaning that via diffusion through the cell membrane it reaches the cell and then the cell nucleus and once there binds to its receptor with high affinity. The VDR belongs to the family of ligand-controlled transcription factors. After being activated by its ligands, VDR modulates the transcription of those gene segments that carry a DNA element assigned to the receptor, so-called hormone response elements (HREs). For the VDR these gene segments are called vitamin D response elements (VDREs). Genes containing VDREs are, for instance, osteocalcin (OC), calbindin D9k (CALB3) and D28k (CALB1), vitamin D 24-hydroxylase (CYP24), osteopontin (OPN), atrial natriuretic peptide (ANP), parathormone (PTH), carbon anhydrase II (CA-II), integrin beta 3 (ITGB3), fibronectin (FN1), c-fos, parathormone related peptide (PTHrP), “slow myosin heavy chain 3” (slowMyHC3), tissue specific plasminogen activator (t-PA), growth factor 1 (PIT1; POU1F) and involucrin (IVL).

The study by DeLuca et al. (147) has shown that the active vitamin form is also present in lymphocytes (CD4 and CD8), especially in activated T lymphocytes. Immune suppression of autoimmune diseases could be traced back to the active form of vitamin D₃ and Ca²⁺. Given that it could be shown as part of the present invention that diabetes incidence in BB.6S rats could be increased from 15 to 45% if a Ca²⁺ rich diet was used, there must be another regulatory mechanism of YY1 at work here.

Guo et al. (148) were the first to be able to demonstrate the role of YY1 in the calcium metabolism for the osteocalcin gene. YY1 represses transactivation of the bone osteocalcin gene mediated by 1.25-dihydroxycholecalciferol. Based on its interaction with the two components, VDR and transcription factor IIB (TFIIB), YY1 is able to regulate vitamin D dependent transcriptions. In addition the VDR/RXR (retinoid X receptor) heterodimer competes with YY1 for the binding sites of the VDR elements in the osteocalcin gene. Because VDREs are present in many genes, YY1 takes on additional regulatory roles there.

In addition, it is possible for YY1 to intervene directly in calciferol synthesis. As a result of transcription repression of the enzyme 25-hydroxyvitamin D3 24-hydroxylase [24 (OH)ase; CYP24], the catabolism is interrupted. Enhanced repression was observed with TFIIB or CBP (CREB-binding protein) present (149). In addition, in transgenic rats in which CYP24 was overexpressed, it was demonstrated that these animals developed almubininuria and hyperlipidaemia. This is an observation that in this connection reminds one of the increase in blood fat of BB.6S rats compared to the parental strain of BB/OK rats. These transgenic rats also developed atherosclerotic lesions of the aorta (150).

Chang et al. (151) have been able to show that the VDR is connected with type 1 diabetes. The active form of vitamin D also acts as a direct negative factor in the renin-angiotensin system. This system plays an important role in the regulation of fluid and electrolyte balance and therefore also in controlling blood pressure. If there is fluctuation of the vitamin D level, there is direct inhibition of renin gene expression. Thus vitamin D does not just play a role as regulator in maintaining calcium haemostasis but also in haemostasis of the electrolytes, the blood volume and blood pressure (152). A study that lends support to this is that of Bhalla et al. (153). In this it was shown that YY1 activates “brain natriuretic” peptide (BNP) gene transcription via CBP/p300 in synergy with GATA-4 as transcriptional complex. BNP belongs to the family of natriuretic peptides that consists of 3 peptides (atrial natriuretic peptide, ANP; BNP; natriuretic peptide type C, CNP). Their primary effect is to increase sodium excretion, bring about widening of the blood vessels (vasodilation) and a decrease in aldosterone secretion (the most important mineralocorticoid, influencing sodium, potassium and water balance in all tissues), making them the effective counterpart of the renin-angiotensin aldosterone system. GATA-4 belongs to the family of GATA zinc finger transcription factors and is expressed in adult heart tissue, intestinal epithelial cells and gonads. During foetal development GATA-4 plays a role in heart formation. This is why GATA-4 and YY1 function equally as a key element in myocardial differentiation and function. Both influence numerous heart genes (154-158). Amongst other things, it could be shown that YY1 is linked to hypertrophy of cardiac myocytes (mediated via IL1β) (159).

In view of the research hitherto available, as well as our own results (see above), it may be concluded that YY1 itself intervenes in calcium metabolism or else is influenced by calcium (and their components). Apart from that, it may be assumed that YY1 plays an essential role in regulating blood pressure and muscle contraction. This is why YY1 should be downregulated or upregulated depending on the tissue in question and sex.

Using mouse embryos it has been shown that YY1 has an important role in the development of bones. It is able to activate the MSX2 gene (160). MSX2 belongs to the class of homeobox genes and is expressed in numerous embryonal tissues. MSX2 plays a role as a key mediator in many different development processes, such as skull, teeth and eye formation, as well as in craniofacial morphogenesis. It is involved in epithelial-mesenchymal interaction and apoptosis (161-165). During dysregulation of the expression level of Mxs2 abnormal growth was noted. Two factors, BMP4 (bone morphogenetic protein type 4) and YY1, regulate the expression of MSX2 in embryonal tissues (166). Independently of BNP4, YY1 can bind to three sites of the MSX2 promotor and thereby bring about activation (160). The involvement of YY1 in embryogenesis, and neuronal, skeletomuscular and bone development allows one to conclude that YY1 definitely has a determining role in mutation or dysregulation of diseases of the muscles, bones and brain (167). This is why YY1 can also be downregulated or upregulated using this, depending on the tissue in question and sex.

Use of the Findings

1. Diagnostics

DNA Sequence and Gene Expression

Based on the present YY1 sequence, YY1 sequence changes are used for the diagnosis of possible dysfunctions using the primers listed in FIG. 11. For this, genomic DNA and ssDNA from mononuclear blood cells are amplified and sequenced with the primers, or point mutation analyses (SNPs) are performed (cf. Kwok P. Y. SNP genotyping with fluorescence polarisation detection. Hum. Mutat. 19, 2002, 315-323).

In addition, RNA from mononuclear blood cells and tissue is extracted using biopsy (muscle, fatty tissue, skin) or renal and hepatic puncture, transcribed into ssDNA and sequenced (ssDNA transcription), and used for gene expression analysis, as described above (cf. pp.22/23, “Gene Expression Studies”). It is assumed that different expression patterns and splice variants (see shortened zinc finger for BB.6S in liver, pancreas and brain) may occur and give important pointers regarding regulation of YY1 in the tissues and regarding the illness itself.

These tissues come into play because in type 1 diabetes secondary diabetic illnesses can occur that manifest themselves in different tissues (diabetic nephropathy, retinopathy, neuropathy etc.). Secondary illnesses are, for example, lipometabolic disorders (fatty tissue, liver), high blood pressure (kidney), cardiovascular diseases (muscle tissue), as well as dermatological illnesses (skin). Because not all diabetics suffer equally from all secondary illnesses, it is expected that the expression profile will differ from proband to proband.

ELISA (Enzyme-Linked Immunosorbent Assay)

So as to be able to perform serological tests for the purposes of diagnosis, the sequence can be used to establish various ELISA procedures. For this monoclonal and polyclonal antibodies are generated.

Manufacture of Polyclonal and Monoclonal Antibodies (See Ref. 89)

In accordance with the present invention, polyclonal antibodies against YY1 are manufactured. Rabbits may be used in their manufacture.

YY1 protein, synthetically and individually manufactured for the proband, is emulsified with what is known as Freund's complete adjuvant. The adjuvant protects the YY1 protein from degrading. Freund's complete adjuvant consists of a mixture of paraffin oil and mannide monooleate, to which inactivated and dried tuberculosis bacteria (mycobacterium tuberculosis) have been added. This results in general immune reactivity, the primary immune response, in the animal. Injection of the mixture into the rabbit is intradermal, subcutaneous or intramuscular. After around four to six weeks the same protein is reinjected with incomplete adjuvant (no bacteria), to trigger a secondary immune response. Depending on the antibody titre, the animal is fully exsanguinated and the antiserum extracted.

For production of monoclonal antibodies (mABs) the hybridoma technique developed by Köhler and Milstein is used.

Mice are immunised with the YY1 protein, manufactured synthetically based on the DNA sequence of the proband. The antibody-producing spleen lymphocytes are then fused in culture with myeloma cells (cancer cells) in the presence of polyethylene glycol. After fusion the so-called hybridoma are distributed in microtitre plates (0.2 ml volume) and cultivated with the HAT (hypoxanthine-aminopterin-thymidine) selection medium. The medium ensures that there is only growth of hybridoma. After 10 days of culture the hybridoma are separated out and propagated on in culture. The mABs are isolated from the cell-culture supernatants.

Antibody Purification (Affinity Chromatography) (See Ref. 89)

Since antisera, along with the specific immunoglobulins that are directed against YY1, contain other antibodies as well, purification is to be performed using affinity chromatography. Here YY1 is bound to a solid matrix. Only antibodies that are specifically directed against YY1 will bind. The other antibodies pass through the column. The specific antibodies are eluted from YY1 by changing the pH value (to 2.5 or over 11).

The specifically purified antibodies to YY1 can then be used in subsequent stages in the ELISA procedure or Western blot.

The subject of the present invention are therefore also antibodies that are directed against an aforementioned protein or peptide of the invention. The antibodies in question are preferably monoclonal antibodies.

In this connection, sandwich ELISAs can also be used to detect antigens, and detection of specific antibodies to an antigen using enzyme-marked secondary antibodies is also suitable.

Establishing ELISA Procedures (See Ref. 89)

Detecting specific antibodies to YY1 using enzyme-marked secondary antibodies

Synthetically manufactured YY1 proteins are coupled to a solid carrier (plastic surfaces, microtitre plate). Next, a coating of the proband material to be examined (serum) is applied. There is binding of specific antibodies in the serum that are directed against YY1. Unbound components are eluted. Using a secondary antibody, chemically labelled with an enzyme, the substrate is converted during colour reaction. Using a standard which is carried along, direct correlation with the antibody concentration is possible once the calibration curve has been established.

Sandwich ELISA (Antigen Detection) (See Ref. 89)

The antibodies that are specifically directed against YY1 are bound to a solid carrier (plastic surfaces, microtitre plate). Next a coating of the proband material is applied. The YY1 proteins bind specifically to the antibody. Unbound components are eluted. Using monoclonal antibodies and an anti-antibody (enzyme labelled) the amount of YY1 proteins is determined.

Western Blot (See Ref. 144)

It is assumed that YY1 proteins differ as to size (molecular weight) between the probands, this being determined by alternative splicing or else being already genetically determined.

For this reason, YY1 proteins can be separated by size using SDS-polyacrylamide gelelectrophoresis (SDS-PAGE). The proteins acquire negative charges via the SDS (SDS-binding). After gelelectrophoresis, the separation gel is transferred onto an immobilised membrane (nitrocellulose). The proteins are stained using alkaline phosphatase and specific antibodies directed against YY1.

The subject of the invention is therefore a method to determine the tendency of falling ill with type 1 diabetes, autoimmune diseases in general (particularly arthritis, multiple sclerosis, autoimmune encephalomyelopathy, celiac disease), type 2 diabetes, cancer (particularly lung cancer, breast cancer, and colo-rectal cancer etc) or disorders of the mineral and lipoid metabolism, where genomic DNA is amplified from isolated mononuclear blood cells, and sequenced, and where alterations or modifications of the nucleic acid sequence are determined, said sequence encoding a protein having the amino acid sequence illustrated in SEQ ID NO:6, where deviations of codons (non-silent mutations) display increased tendency for falling ill from one of the aforementioned diseases.

Other mutations that can point to increased tendency to diabetes are in the following sequence regions and can be detected using the following primers: Intron 4: primers: K815-F/K817-R; K815-F/K875; K821-F/K817-R; K821-F/K870-R; K874-F/K870-R. For amplification, preferably the following primers are used: K823-F/K825-R; K884-F/K806-R; K801-F/K804-R; K814-F/KK832-R; K828-F/K833-R; K831-F/K817-R; K815-F/K870-R; K815-F/K818-R; K816-F/K819-R; K834-F/K836-R, F15/R12; F15/R14; F15/R13; F57/R16; F57/R20; F57/R21; F59/RR25; F59/RR30; F59/R33; F95/R34; F96/R39; F95/R48; F95/R50; F60/R7; F60/R8; F60/R66; F60/R67; F96/R76; F96/R77; F96/R81 F96/R83; F33/R1; F33/R4; F33/R15; F39/R28; F40/R3; F41/R5; or other combinations.

Also included is a method to determine the tendency of falling ill with type 1 diabetes, autoimmune diseases in general (particularly arthritis, multiple sclerosis, autoimmune encephalomyelopathy, celiac disease), type 2 diabetes, cancer (particularly lung cancer, breast cancer, and colo-rectal cancer etc.) or disorders of the mineral and lipoid metabolism, where RNA is isolated from isolated mononuclear blood cells or tissue biopsies (fatty tissue, muscle tissue, skin), or tissue punctures (liver, kidney), amplified and quantified, where enhanced/reduced expression indicates an increased/reduced tendency to fall ill with type 1 diabetes, autoimmune diseases in general (particularly arthritis, multiple sclerosis, autoimmune encephalomyelopathy, celiac disease), type 2 diabetes, cancer (particularly lung cancer, breast cancer, and colo-rectal cancer etc.) or disorders of the mineral and lipoid metabolism. (cf. above under ‘Gene Expression’).

2. Treatment

2.1 Using DNA or Antisense

For downregulation of YY1, DNA or antisense is to be generated from the present sequence using PCR and applied. Both DNA and antisense oligonucleotides are modified so as to enhance their stability with regard to most exo- and endonucleases (168). Application of the stabilised DNA or antisense is subcutaneous (s.c.) or intramuscular (i.m.). The dose depends on individual expression of YY1, which must be checked at certain intervals so as to obtain a good balance of YY1 with regard to other genes.

Antisense Oligonucleotides

Because antisense oligonucleotides have high specificity, they can bind specifically, block RNA, and thereby prevent expression. Antisense oligonucleotides must be distributed at intervals of 2 to 20 bases across the sequence. Preferably one starts at position 73.

Important fragments where several antisense oligonucleotides are produced (always displaced by one position), are: 73 to 564, 565 to 717, 718 to 834, 835 to 1071; 955 to 999, 955 to 1041, 1000 to 1041, 1042-1125, 1759 to 1848, 1849 to 1938, 967 to 1125, 1071 to 1938 and the intron sequence 1126-1758. PCR amplification is as described (12).

Oligonucleotide Modifications

For improving efficiency/stability using:

1.) Methylphosphonates

2.) Phosphorothioates
Nuclease resistant, water soluble, strong hybridization with mRNA
Increases RNase H activity, which is responsible for the reduction of mRNA in the double strand

3.) Phosphodithionate

4.) Polyamide backbone (PNA-DNA chimera) instead of pentose phosphate (Uhlmann E., Peyman A., Breipohl G., Angew. Chem. 1998, 110, 2954-2983.)
Chimera better for cell uptake

For improving transport characteristics using:

1.) Alkylation (increase in hydrophobicity)

2.) Coupling to sugars

Ribozymes

Ribozymes are catalytic RNA, with enzymatic characteristics (phosphate ester hydrolysis; Nobel prize Chech/Altmann). Ribozymes recognise certain base sequences in the mRNA and cut the molecule at these points. This means they have a double function: recognition of a given structure, and then hydrolytic cleavage of a phosphoric diester binding at a given site. Two classes of ribozymes are of particular importance.

These are the ones with a hammerhead structure and others that have a hairpin structure. The endonuclease activity developed by the ribozymes can become effective against practically all RNA structures.

Triple Helix Binding

In addition, a third nucleotide strand is to be bound to a DNA double helix via an additional (Hoogsteen) base pairing. This results in a piece of triple strand that cannot be transcribed further. The positions in the DNA depend on the sequence structure of the probands.

Disadvantage: Recognition is only in purine-rich regions

The subject of the invention is therefore also the use of an aforementioned nucleic acid or antisense oligonucleotide for the preparation of a pharmaceutical composition. The pharmaceutical composition is particularly for the treatment of type 1 diabetes, autoimmune diseases in general (particularly arthritis, multiple sclerosis, autoimmune encephalomyelopathy, celiac disease), type 2 diabetes, cancer (particularly lung cancer, breast cancer, and colo-rectal cancer etc) or disruptions to the mineral and lipoid metabolism.

Moreover, the invention relates to a pharmaceutical composition that contains an aforementioned nucleic acid or an antisense oligonucleotide, as well as, in certain cases, additional pharmaceutically compatible excipients and/or carriers. The compounds are preferably formulated in a form suitable for intravenous (i.v.), subcutaneous (s.c.) or intramuscular (i.m.) application.

2.2 Use of the Protein

YY1 protein or peptides of the same are synthesised in order to be able to be applied. Preferred AA fragments are: 1 to 165, 166 to 215, 216 to 254, 255 to 323, 255 to 302, 255 to 309 (with and without mutation), 310 to 323 (with and without mutation), 324 to 351, 352 to 381, 382 to 411. In addition, the truncated BB.6S region from 299 to 351 is preferred. In this connection, the nucleic acid sequence encoding a protein with the SEQ ID NO:6, and in particular the sequence of SEQ ID NO:5, or fragments thereof, can be brought into an expression vector and expressed under conditions that are suited or advantageous to the chosen vector system. The relevant procedures are well known to the practitioner.

Here modifications of the protein or the peptides by:
a) Acetylations
b) Deacetylations
c) Methylations
d) Phosphorylations
e) O- and N-glycosylations

come to the fore, so as to enhance or to alter stability and activity.

The invention also relates to the use of one or several of the aforementioned proteins and/or peptides for the manufacture of a pharmaceutical composition. The pharmaceutical composition is especially for the treatment of type 1 diabetes, autoimmune diseases in general (particularly arthritis, multiple sclerosis, autoimmune encephalomyelopathy, celiac disease), type 2 diabetes, cancer (particularly lung cancer, breast cancer, and colo-rectal cancer etc) or disorders of the mineral and lipoid metabolism.

Included are also pharmaceutical composition that contain one or several of the aforementioned proteins and/or peptides, where the proteins and peptides may be modified as described above, if required. In certain cases, the composition contain additional pharmaceutically compatible excipients and/or carriers preferably formulated in a form suitable for intravenous (i.v.), subcutaneous (s.c.) or intramuscular (i.m.) application.

Splice Variants

In addition, all possible splice variants (see below) can be identified using the sequence investigated as part of the invention, so as to be able to manufacture a very wide range of proteins. The splice variants of differing size are displayed below. These proteins are preferably modified again (see above), to facilitate a specific application. They can either be used individually or in combination.

Potential Splice Variants (http://www.itba.mi.cnr.it)

DONOR SITES:

| POSITION | EXON | INTRON | SCORE |
|---|---|---|---|
| 157 | GAG | GTGGAG | 78. |
| 379 | GAG | GTGATT | 85. |
| 406 | GAG | GTAGTG | 81. |
| 409 | GTA | GTGGGT | 81. |
| 475 | CCG | GTACCC | 75. |
| 668 | CGG | GTAATA | 80. |
| 694 | CAG | GTGCAG | 78. |
| 1129 | CAG | GTAGAG | 79. |
| 1172 | CTG | GTCAGG | 83. |
| 1214 | GGG | GTATTT | 73. |
| 1273 | CAG | GTGTTA | 77. |
| 1363 | GTA | GTGAGT | 80. |
| 1370 | GTA | GTGTGT | 72. |
| 1423 | CAG | GTGACA | 84. |
| 1453 | CTC | GTGAGT | 79. |
| 1605 | CCA | GTGTGT | 78. |
| 1671 | ATA | GTAGGT | 80. |
| 1675 | TAG | GTGGTT | 77. |
| 1693 | GCA | GTGAGC | 79. |
| 2172 | AAG | GTGTTT | 78. |

ACCEPTOR SITES:

| POSITION | INTRON | EXON | SCORE |
|---|---|---|---|
| 31 | CTCCCGCAG | CCCA | 87. |
| 36 | GCAGCCCAG | GAGC | 85. |
| 335 | GCGCTGCAG | CCGC | 78. |
| 747 | GGTCTTCAG | ATGA | 80. |
| 882 | ACCTCTCAG | ACCC | 80. |
| 1036 | CGGTCCCAG | AGTC | 78. |
| 1053 | TCTGTGCAG | AATG | 77. |
| 1274 | GGACTGCAG | GTGT | 80. |
| 1333 | TTCTAGCAG | GTTT | 79. |
| 1365 | GTTTTGTAG | TGAG | 80. |
| 1418 | TGGCTACAG | CTCC | 77. |
| 1424 | CAGCTCCAG | GTGA | 81. |
| 1447 | TGCTTATAG | AAGA | 80. |
| 1510 | ACTTCCTAG | AGTG | 81. |
| 1572 | TTTCTCAAG | AACT | 84. |
| 1713 | GATCCCCAG | GTTC | 80. |
| 1734 | TTTGCCAAG | AGGG | 78. |
| 1763 | CCTTGACAG | TGCA | 85. |
| 1989 | CCTCTTCAG | GAGT | 79. |
| 2037 | TATTTCTAG | GAAG | 83. |

The splice variants are obtained by corresponding restriction enzymes. The use of the splice variants depends on the individual situation of the probands.

Antibodies

In addition, monoclonal antibodies are generated for regions of the YY1 protein and used for downregulation. Here too, the antibodies must be adapted to the individual situation.

2.3 Enhancing YY1 Expression

The full DNA sequence or partial sequences of YY1 (positions 73 to 564, 565 to 717, 718 to 834, 835 to 1071; 955 to 999, 955 to 1041, 1000 to 1041, 1042-1125, 1759 to 1848, 1849 to 1938, 967 to 1125, 1071 to 1938, as well as the intron sequence 1126-1758), which depend on the individual situation of the proband, are cloned into plasmids under the control of eukaryotic tissue-specific promoters and applied for the long-term expression of YY1 itself or its partial sequences, so as to achieve tissue-specific enhancement of the expression of YY1 or its partial sequences. The vector systems are used according to the state of the art (169).

2.4. Regulation of Other Genes by YY1 Antisense

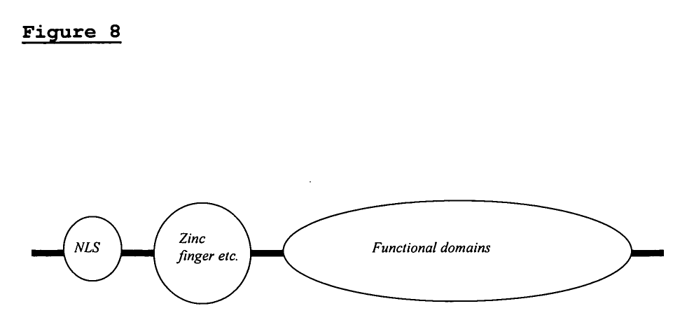

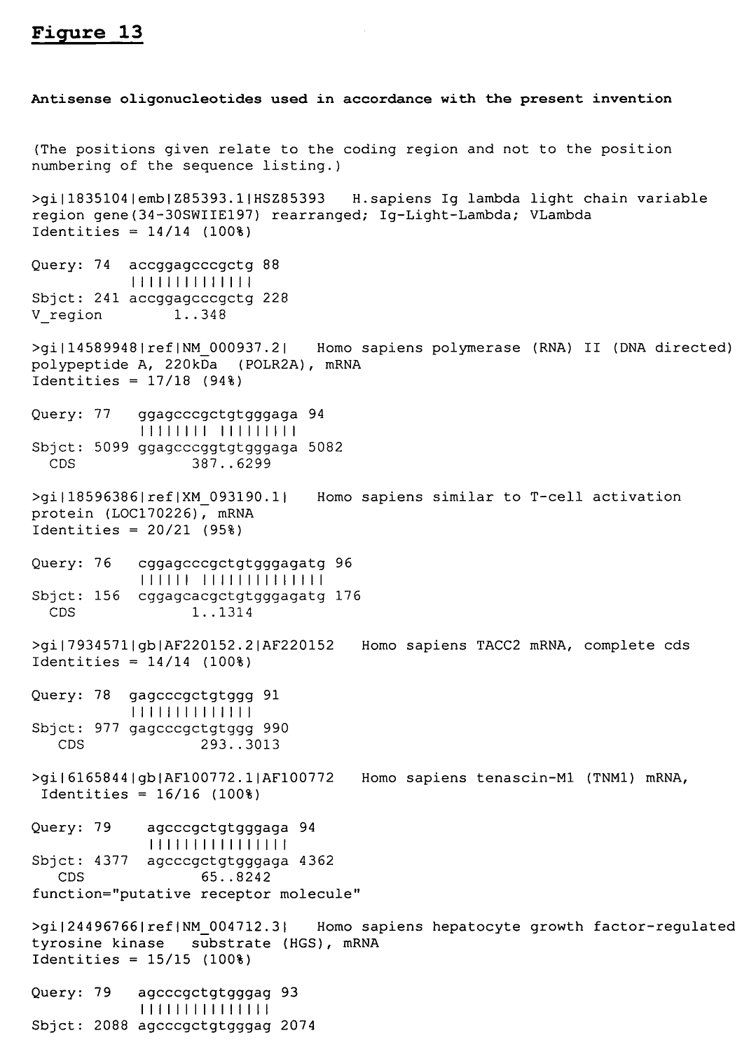

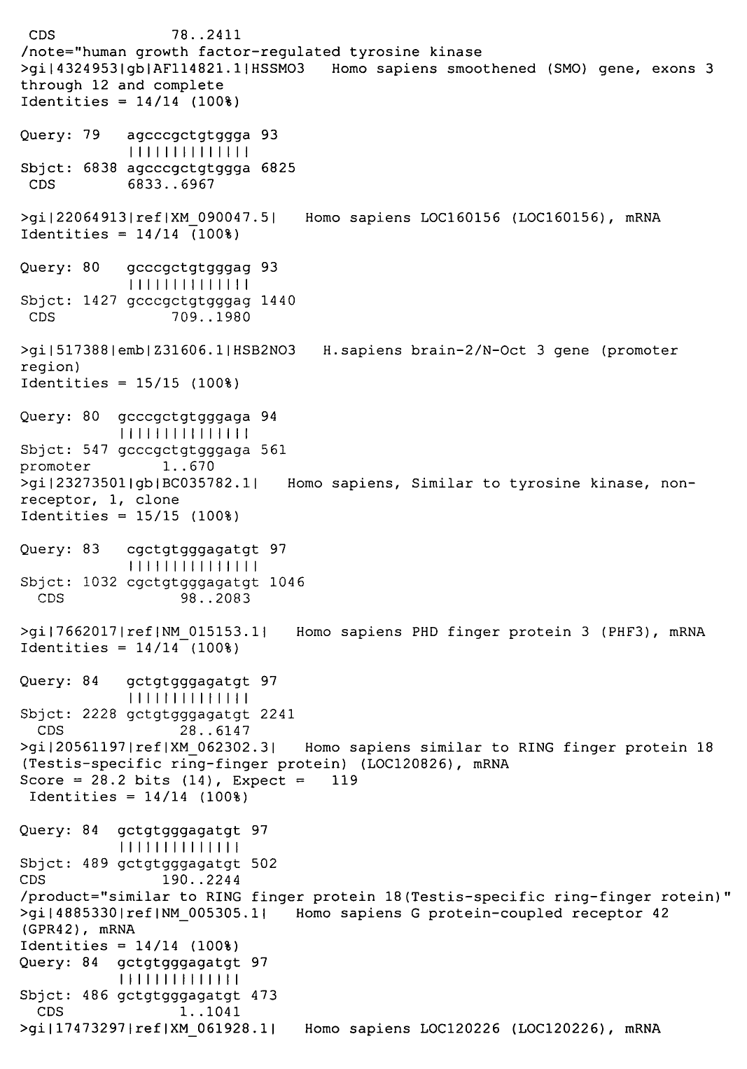

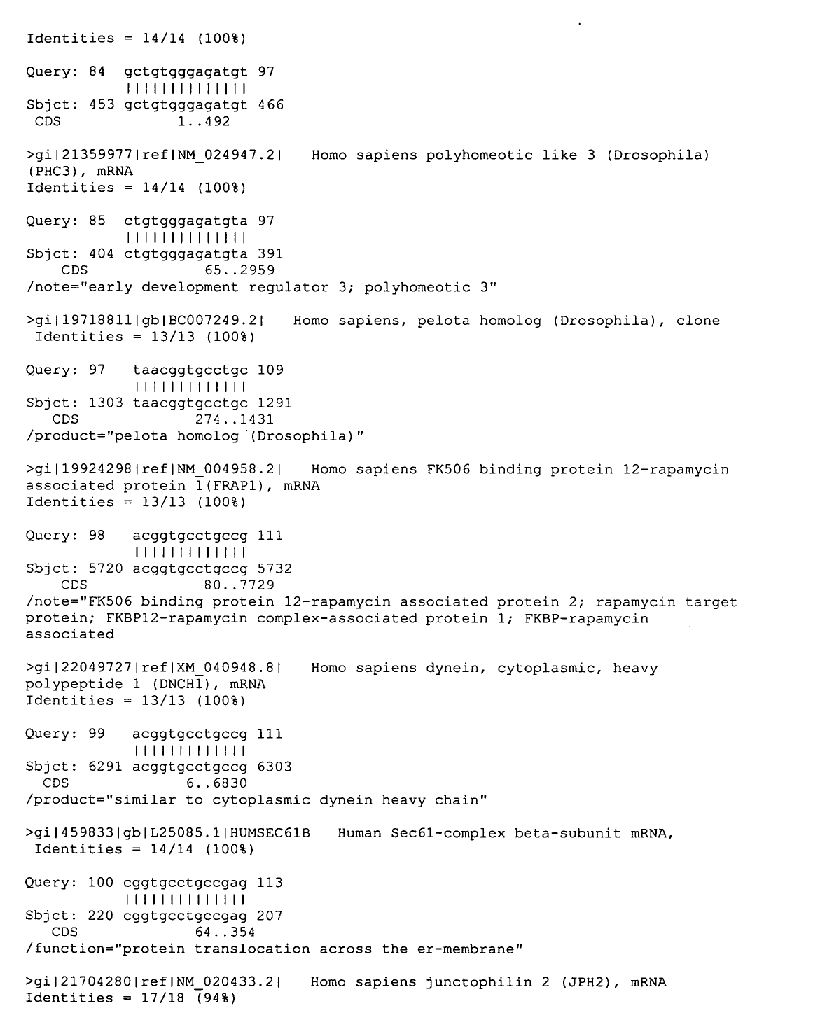

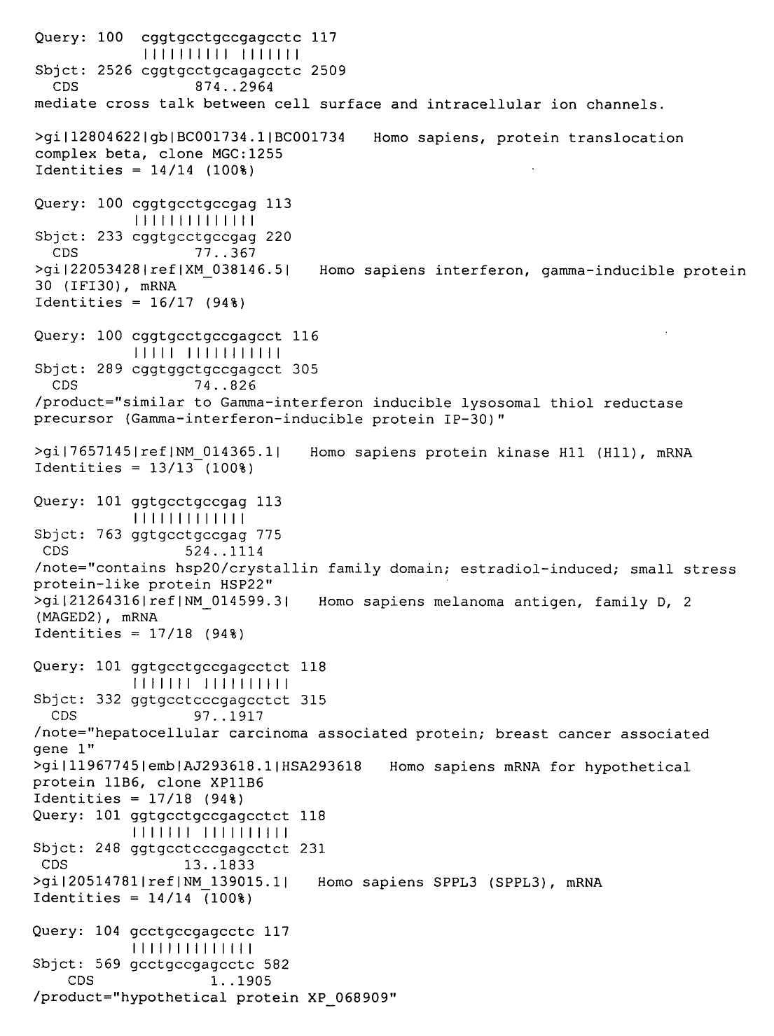

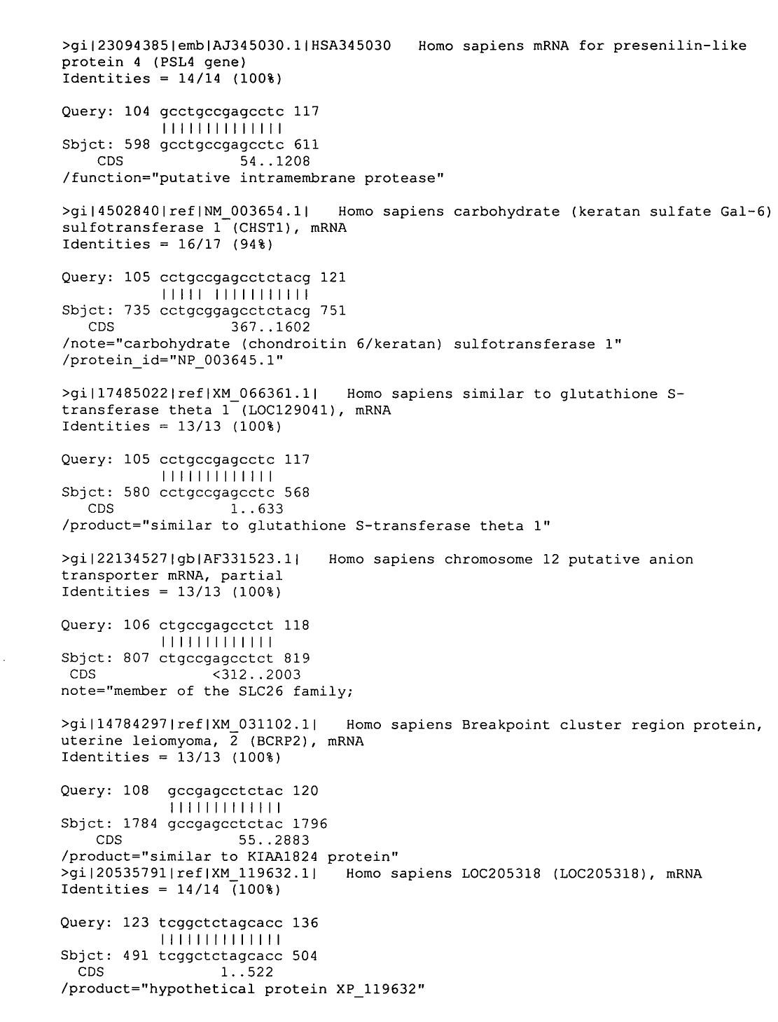

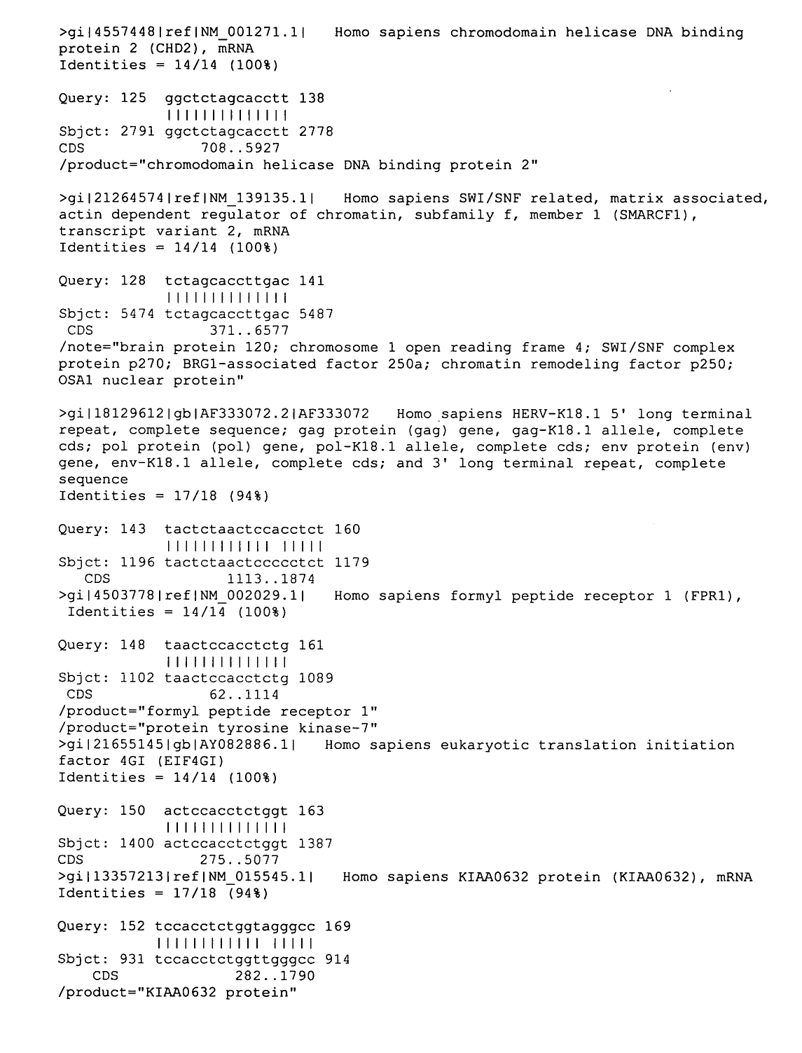

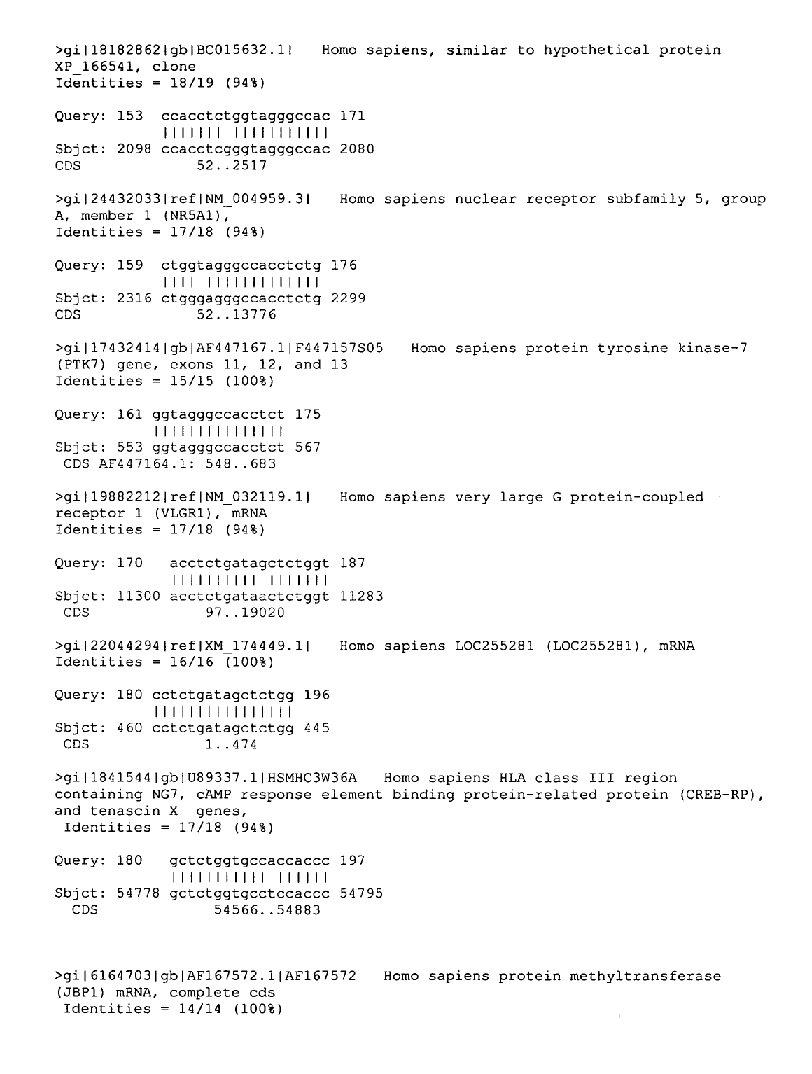

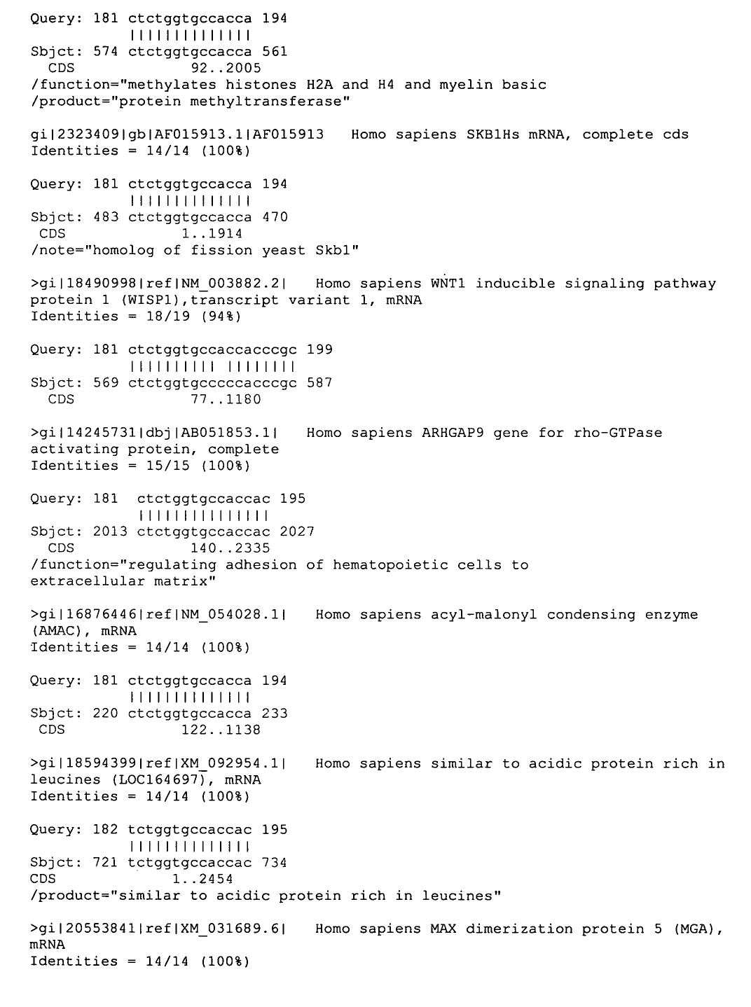

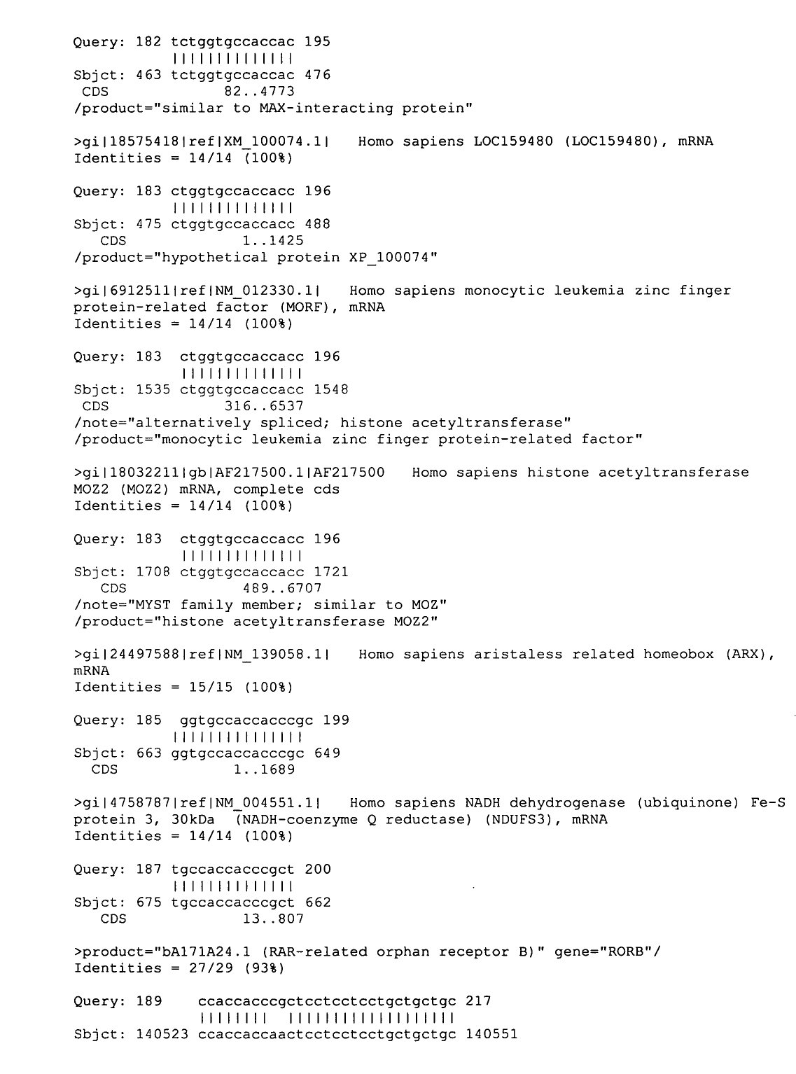

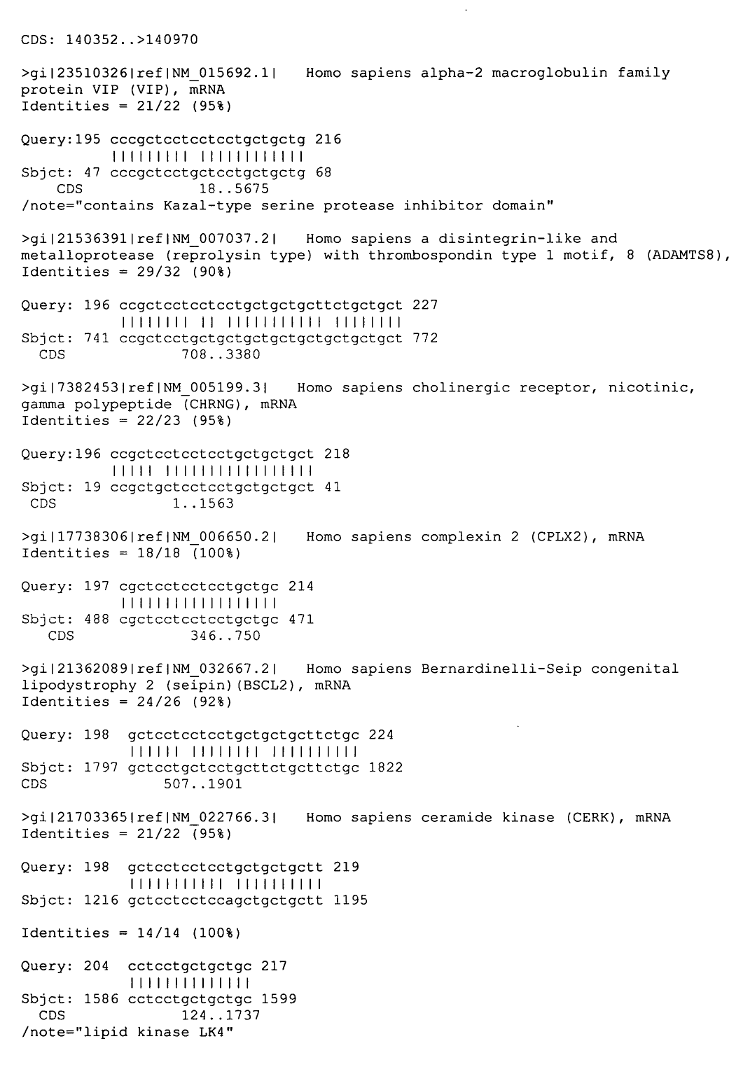

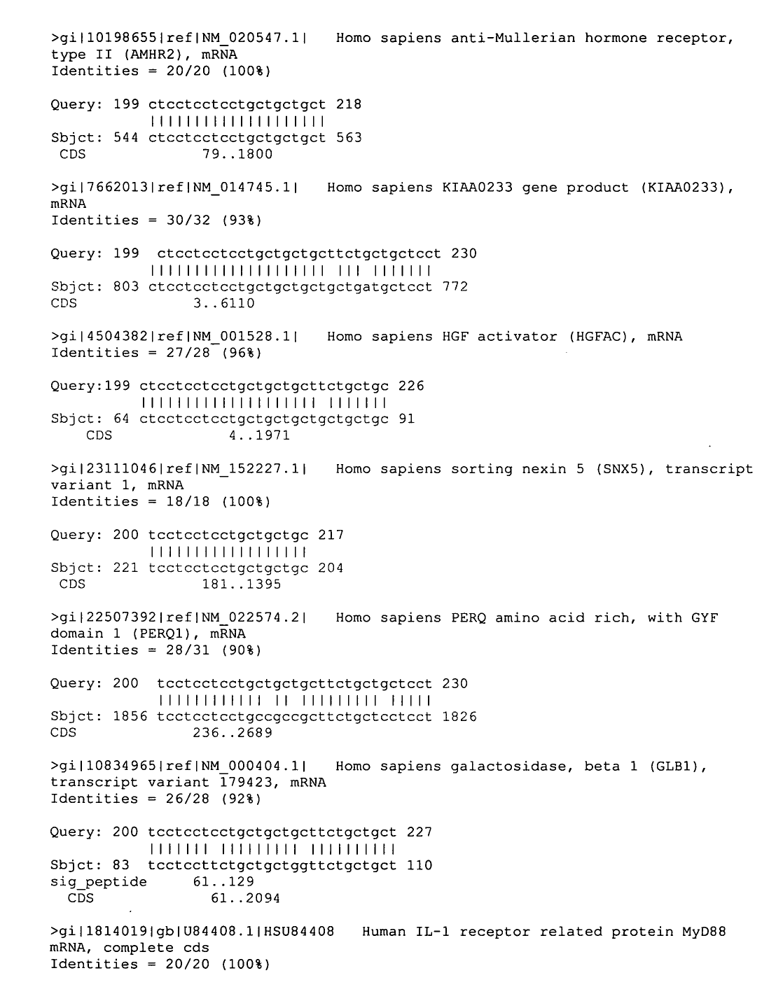

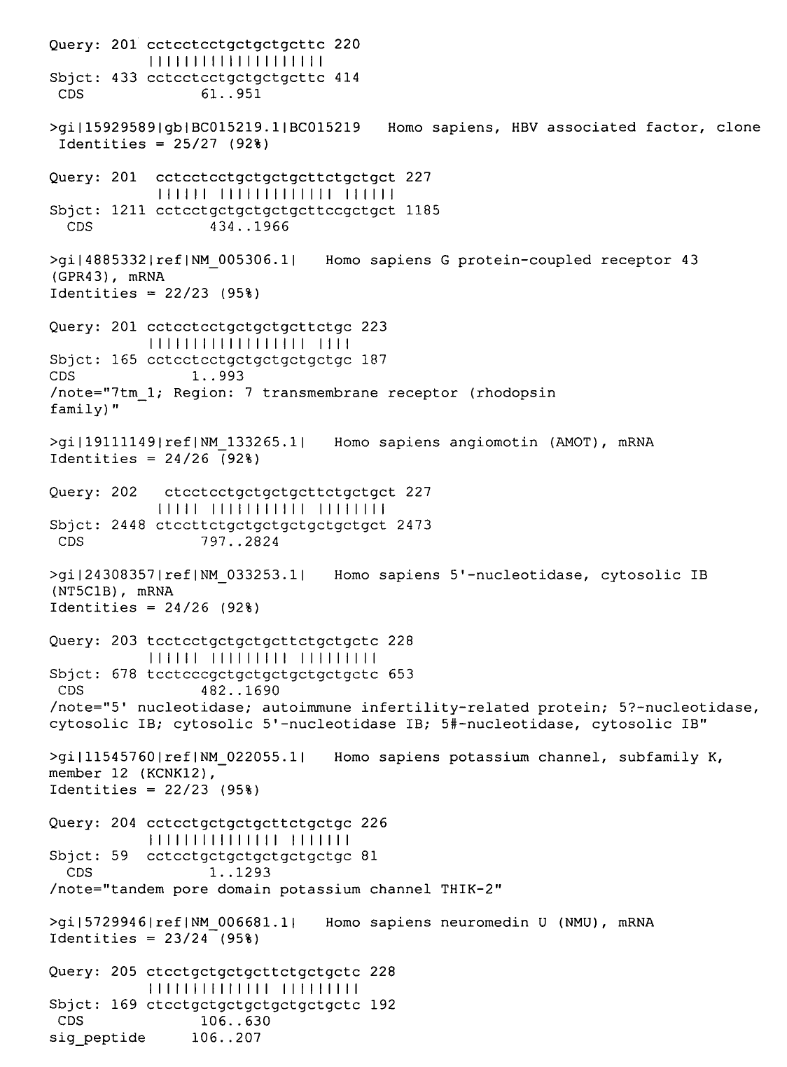

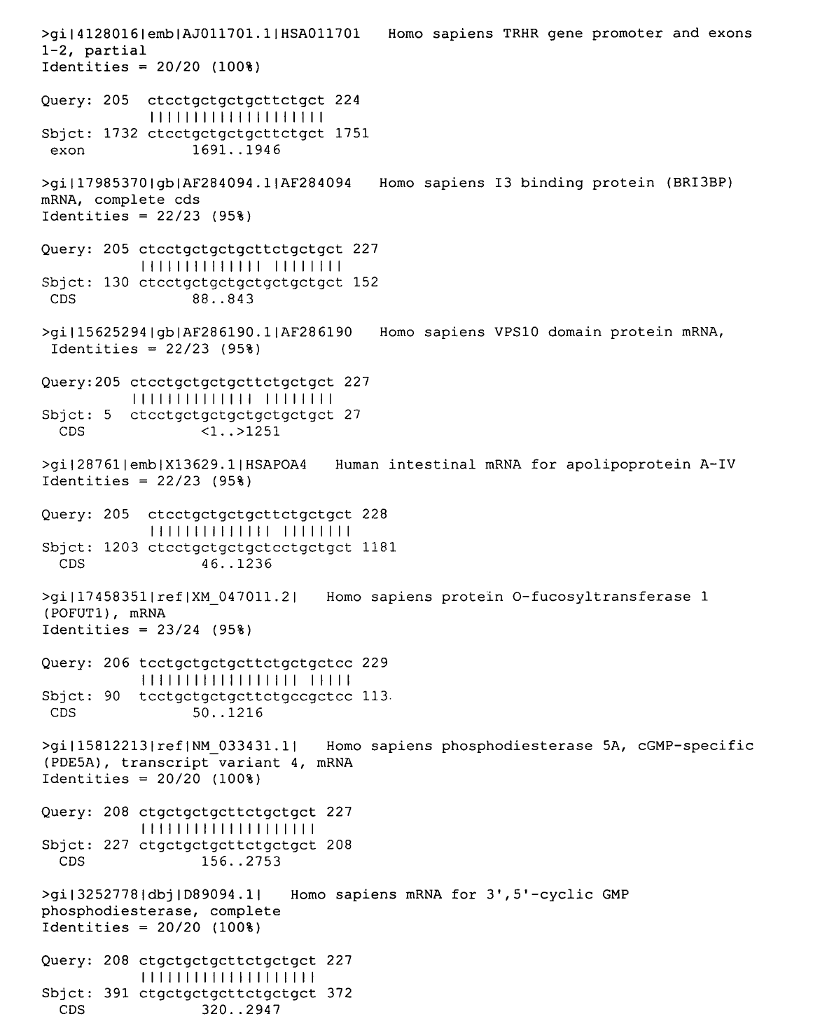

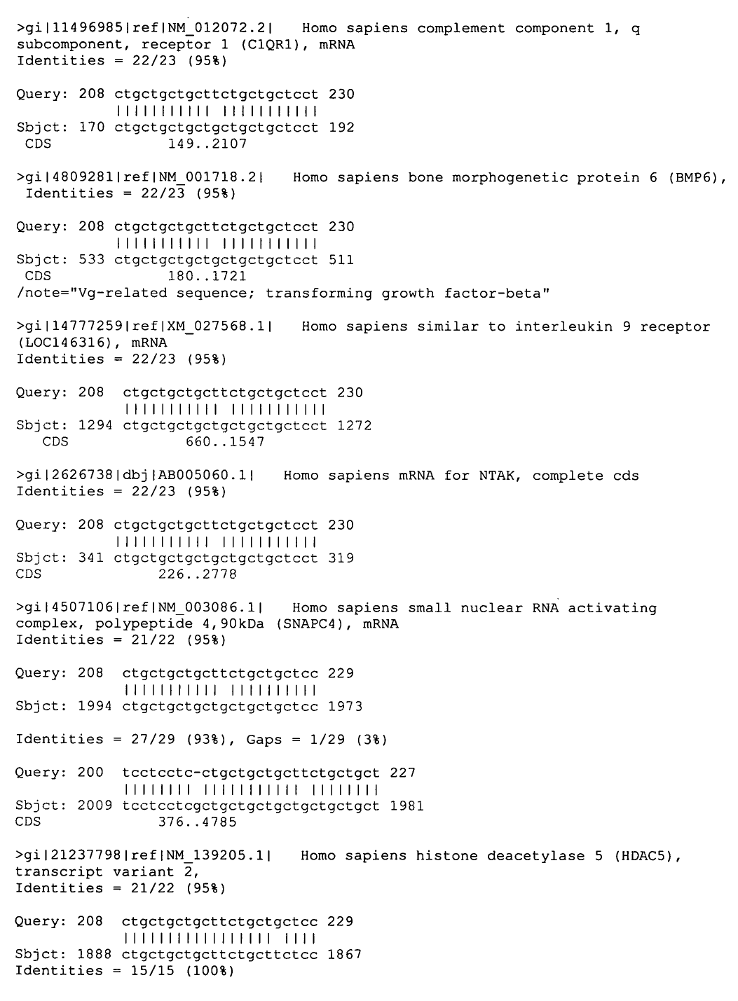

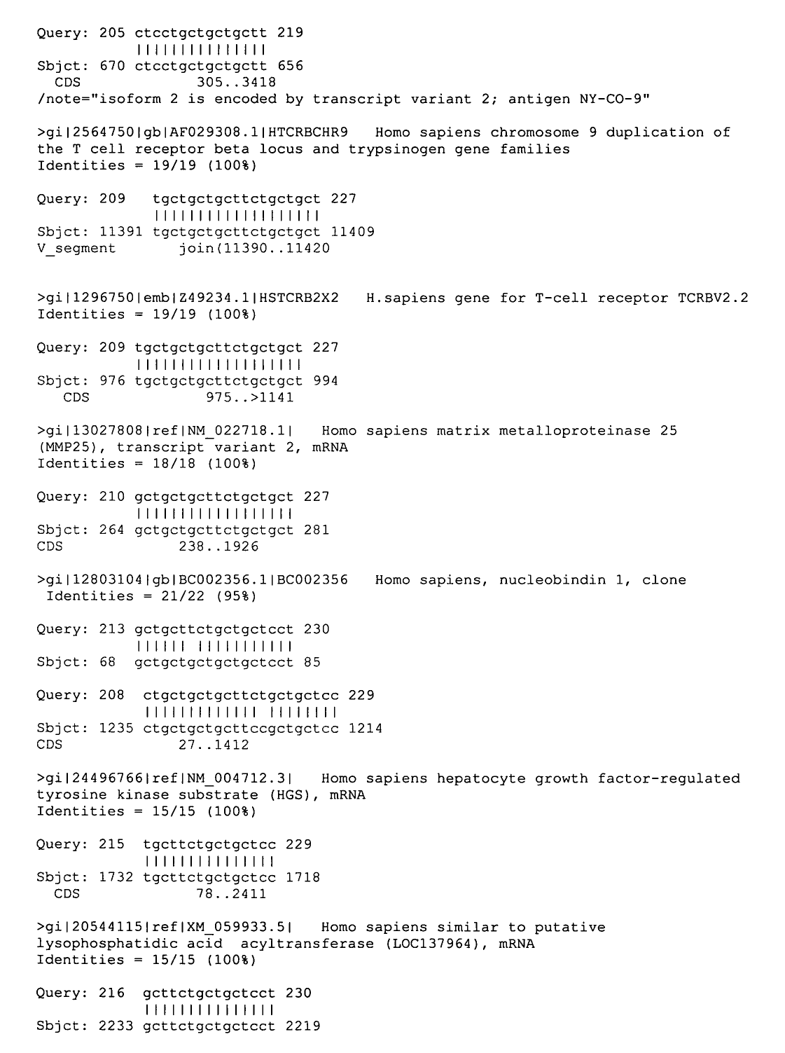

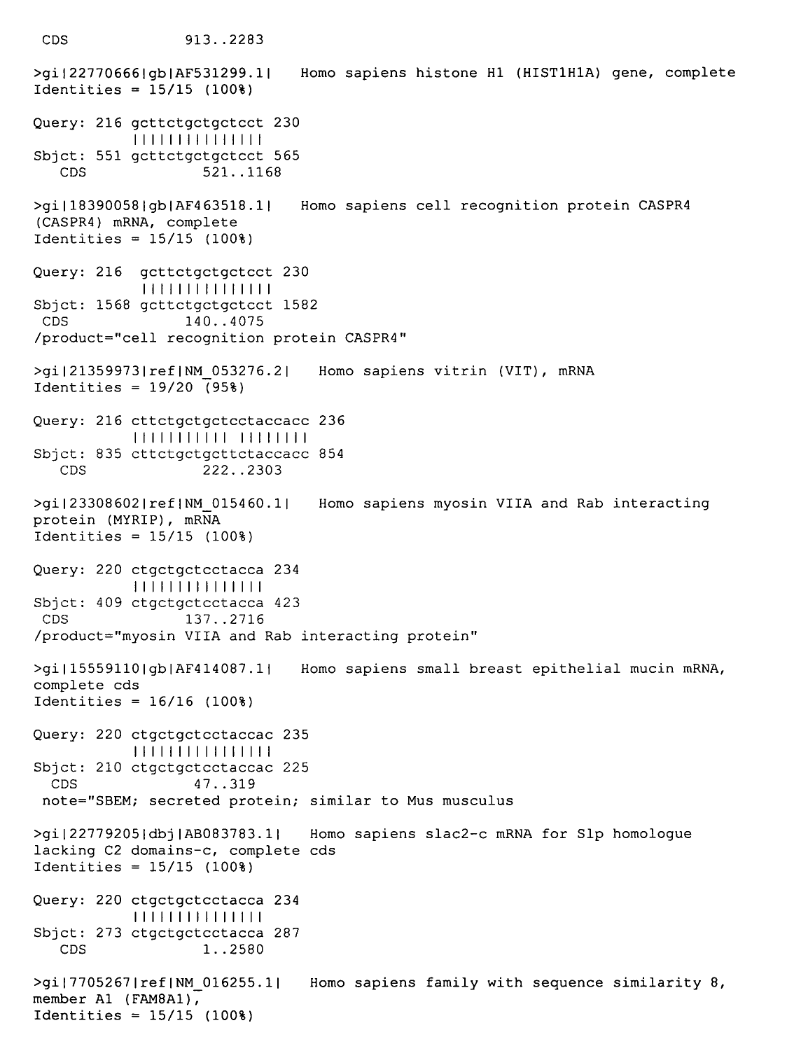

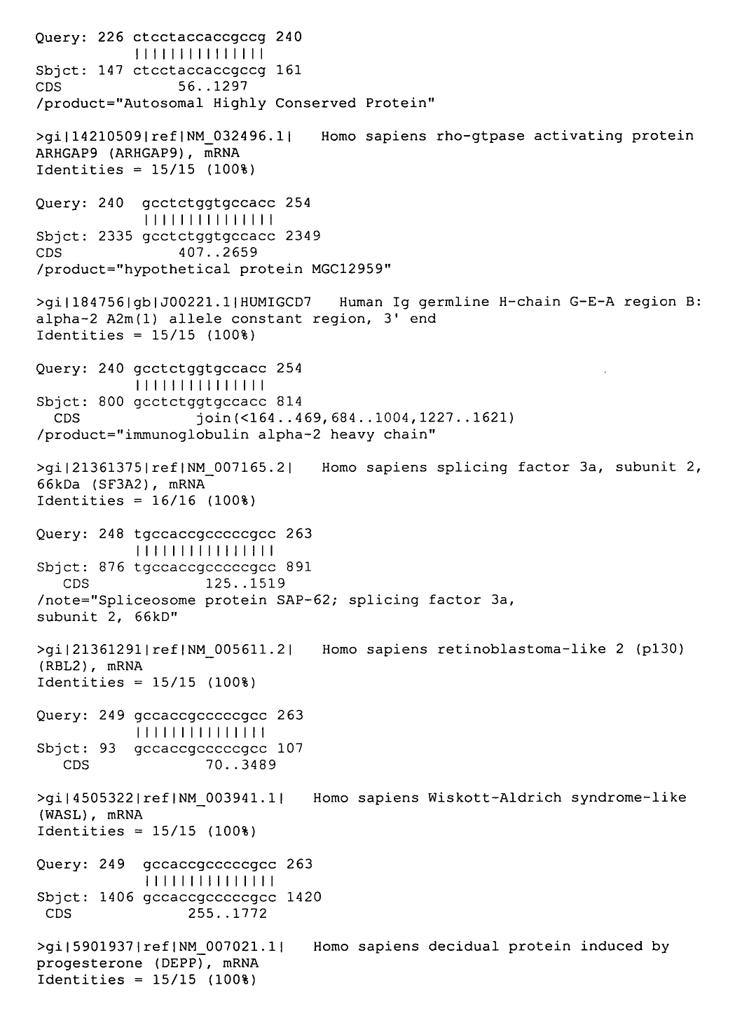

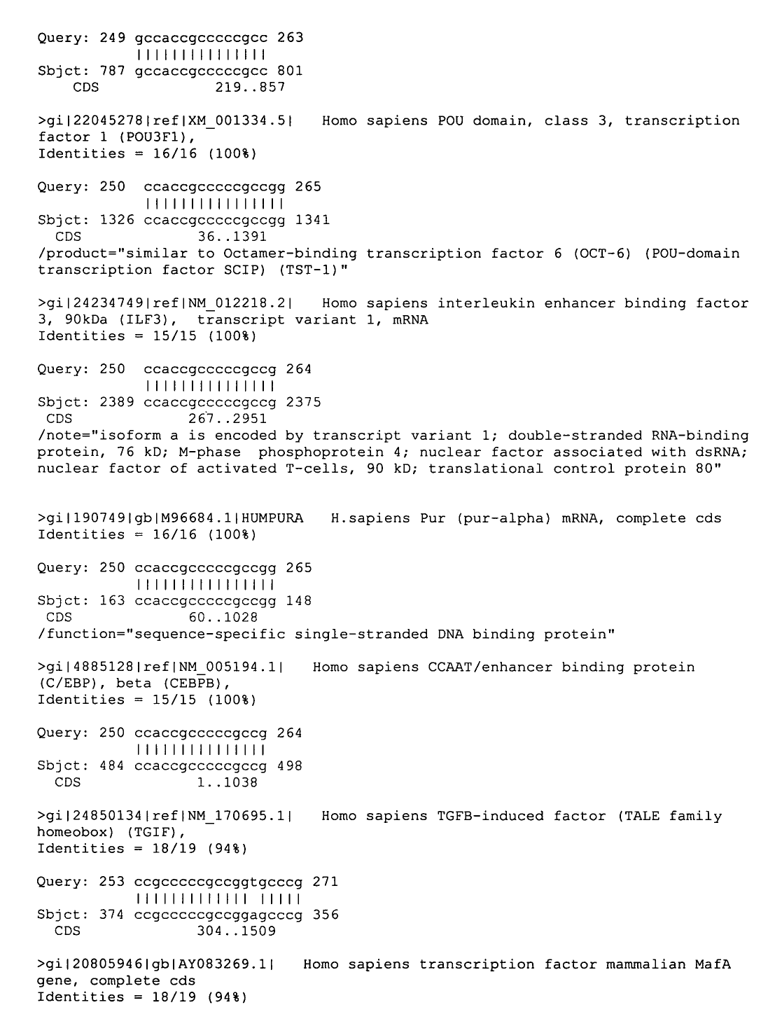

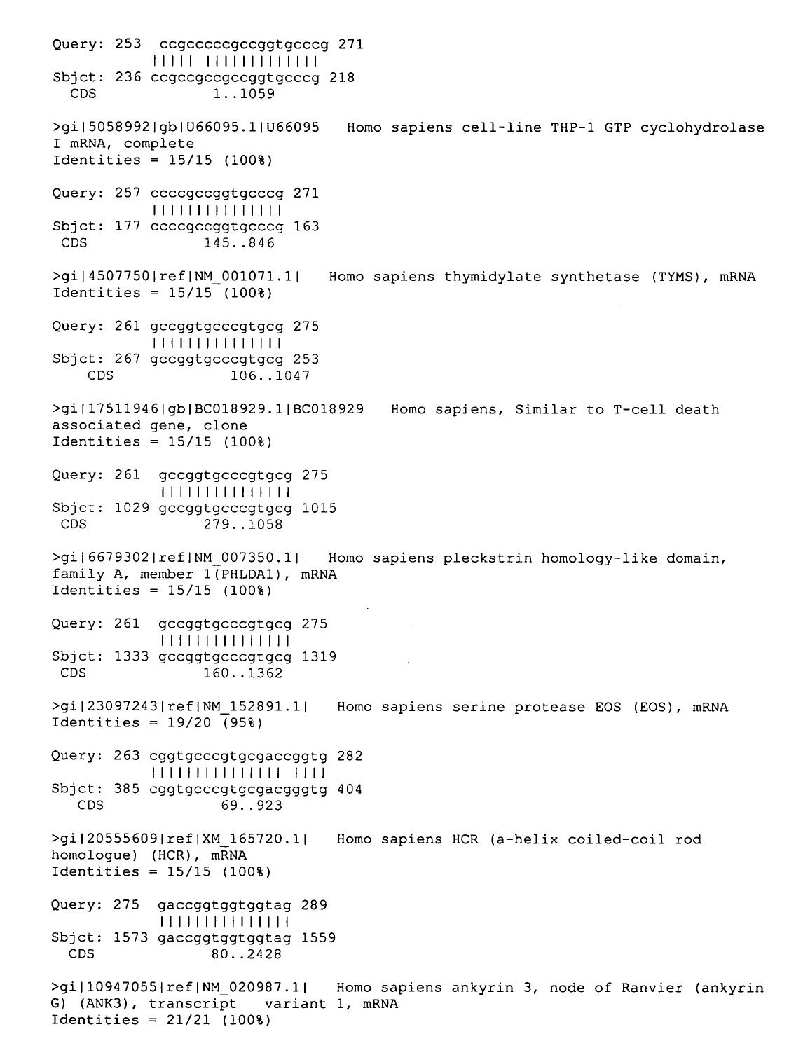

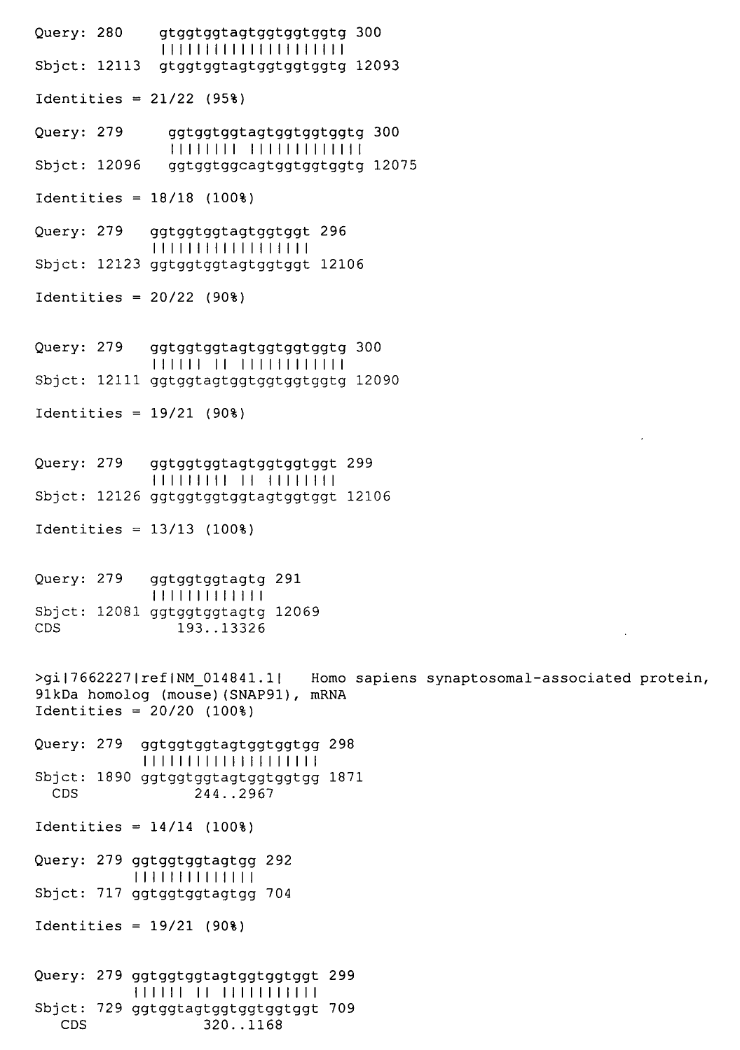

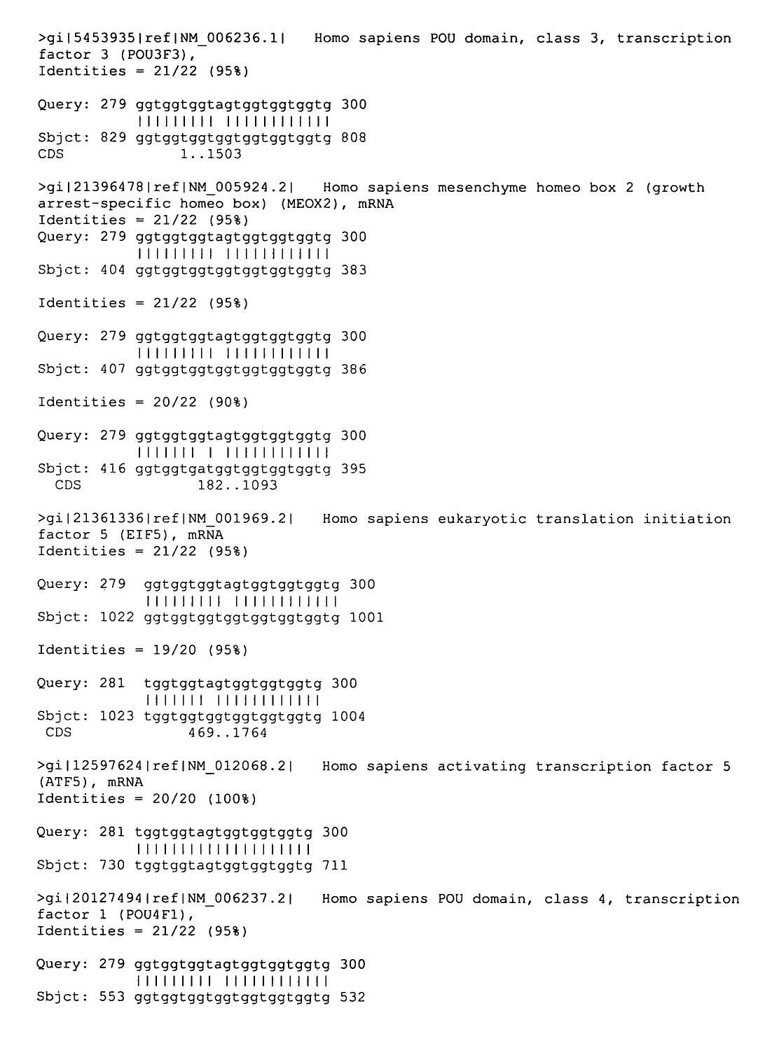

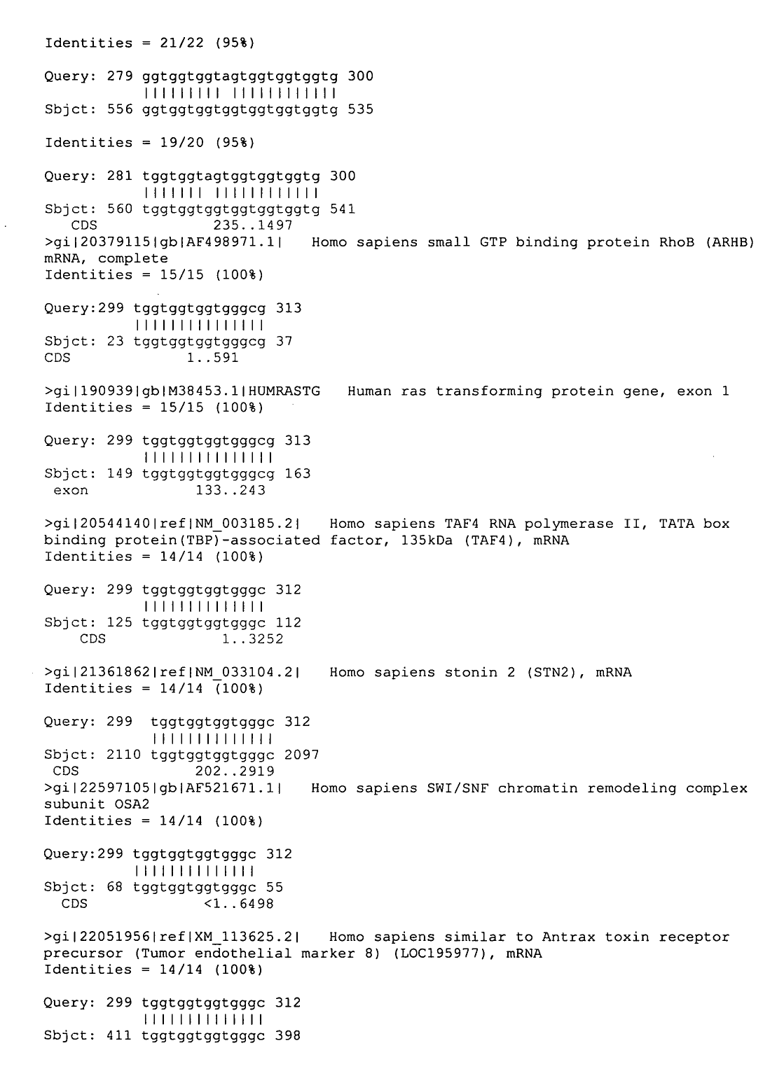

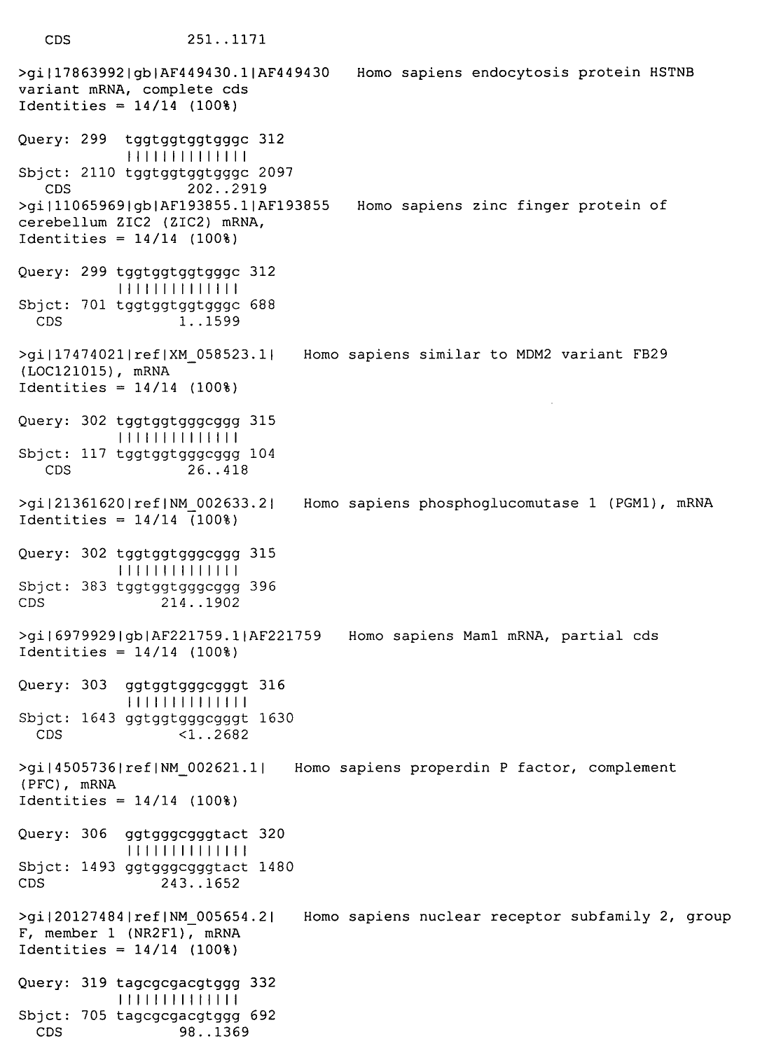

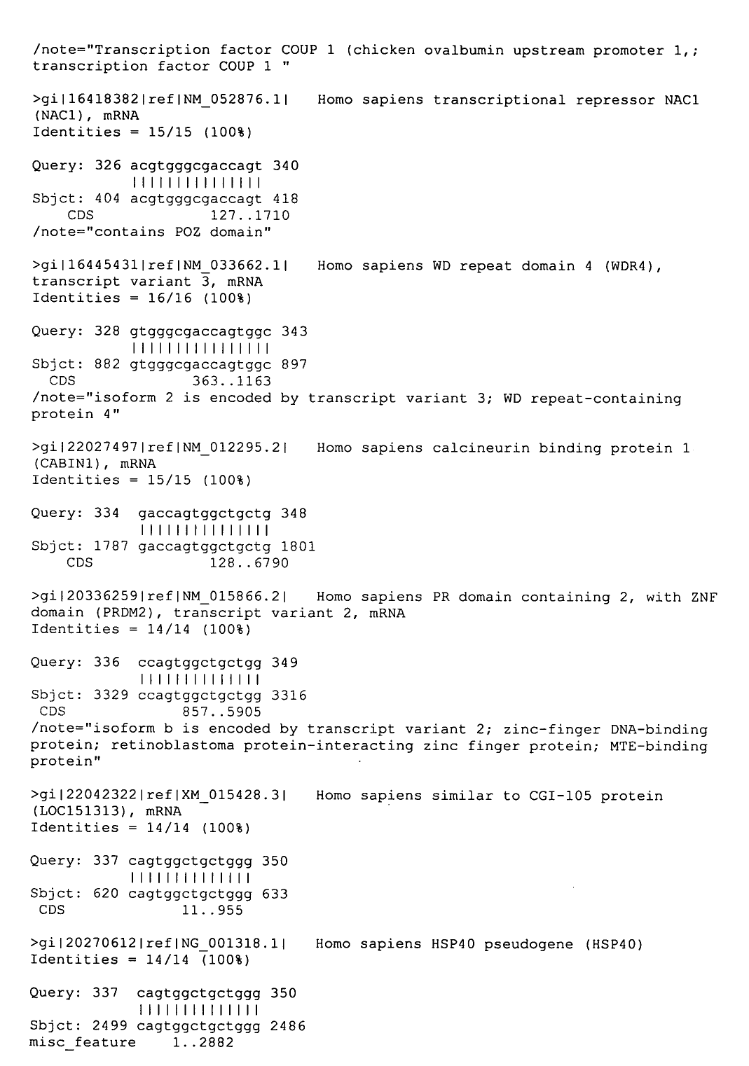

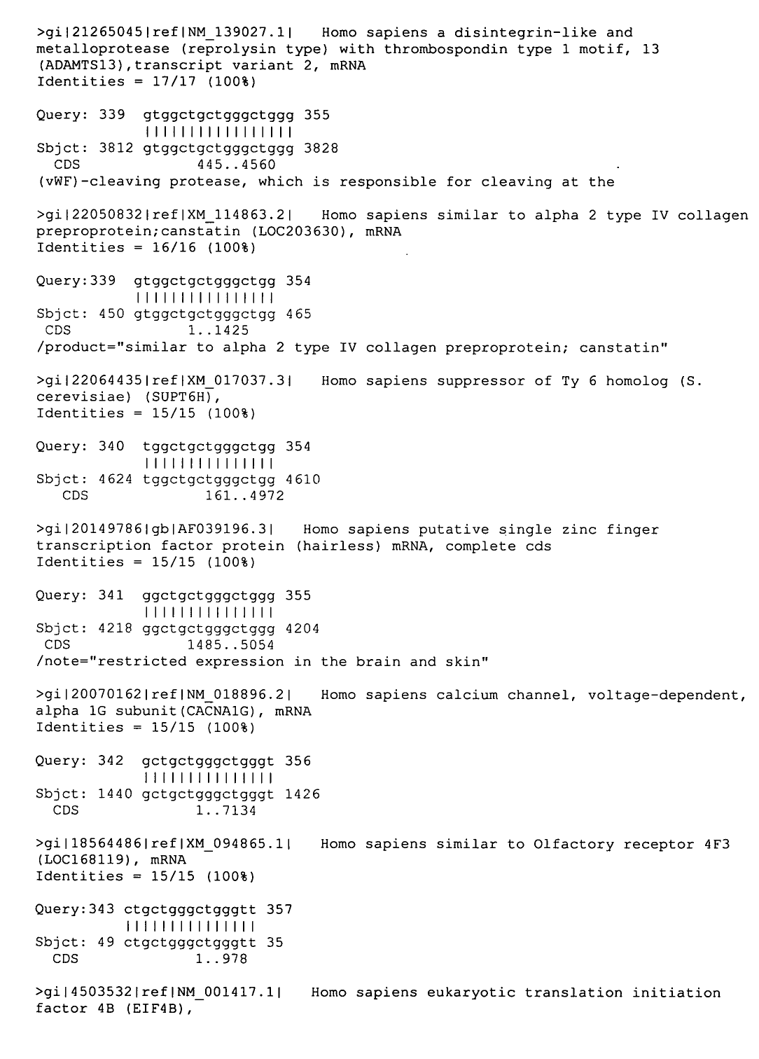

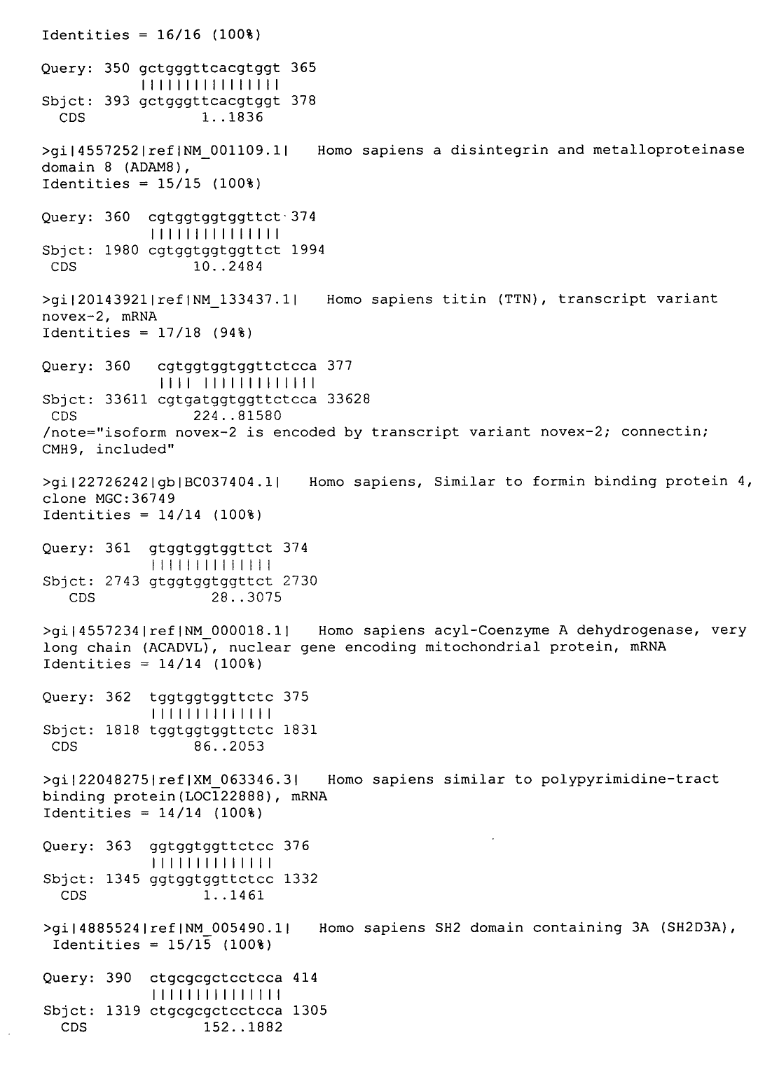

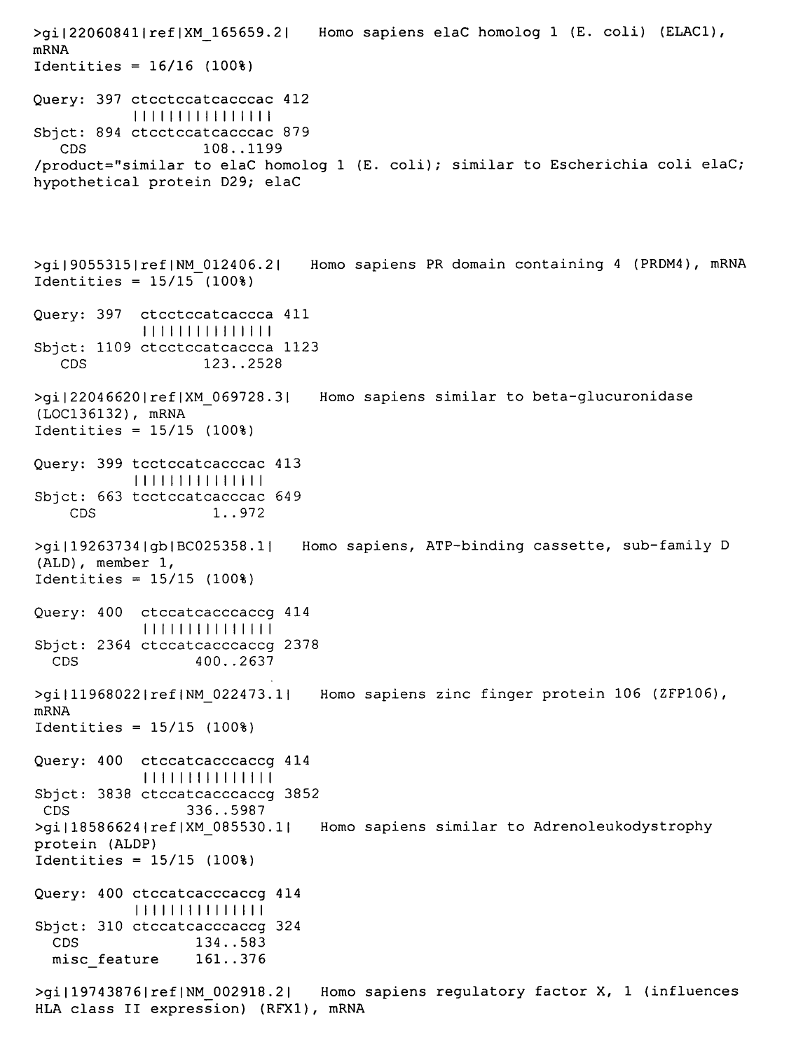

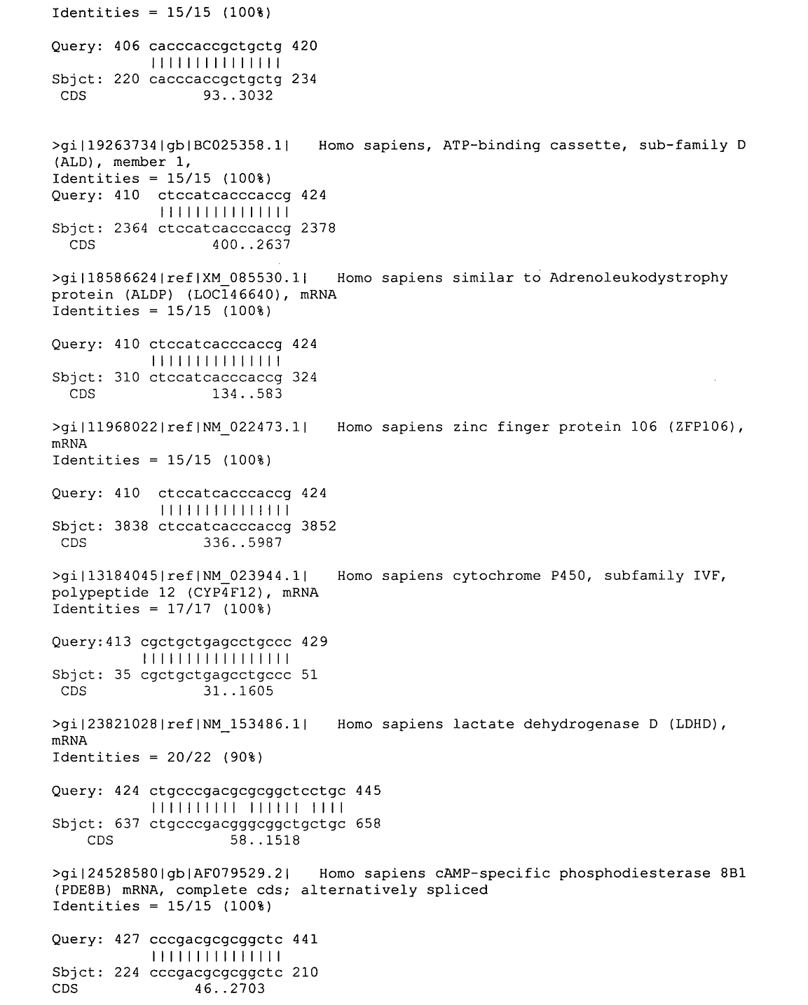

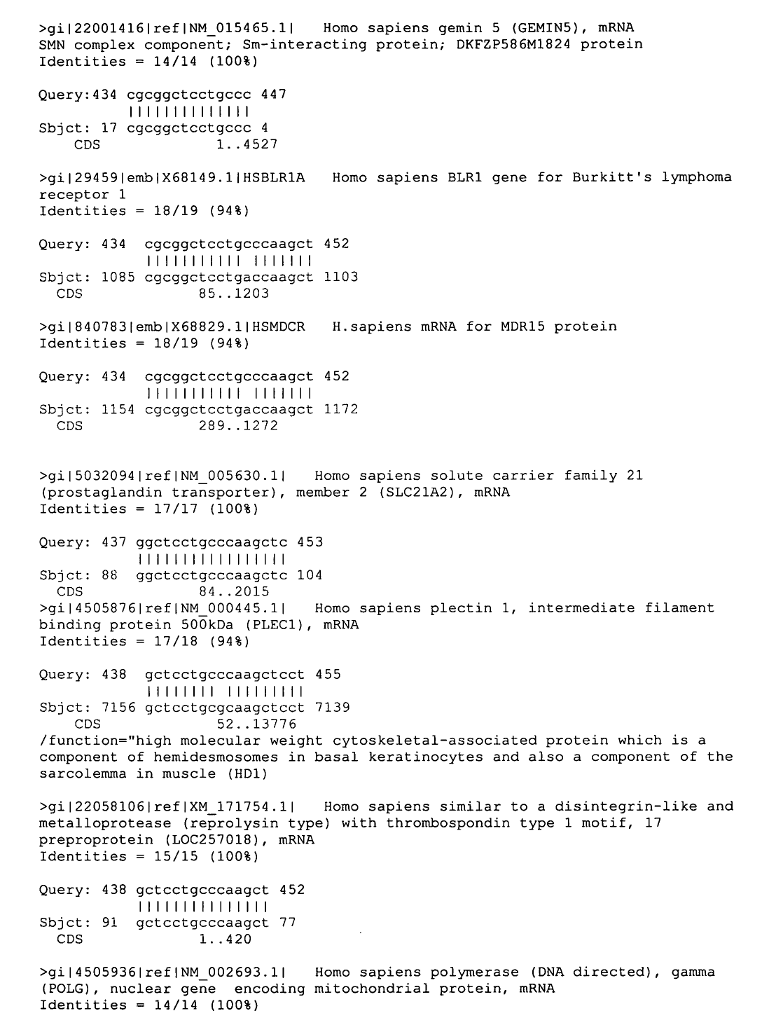

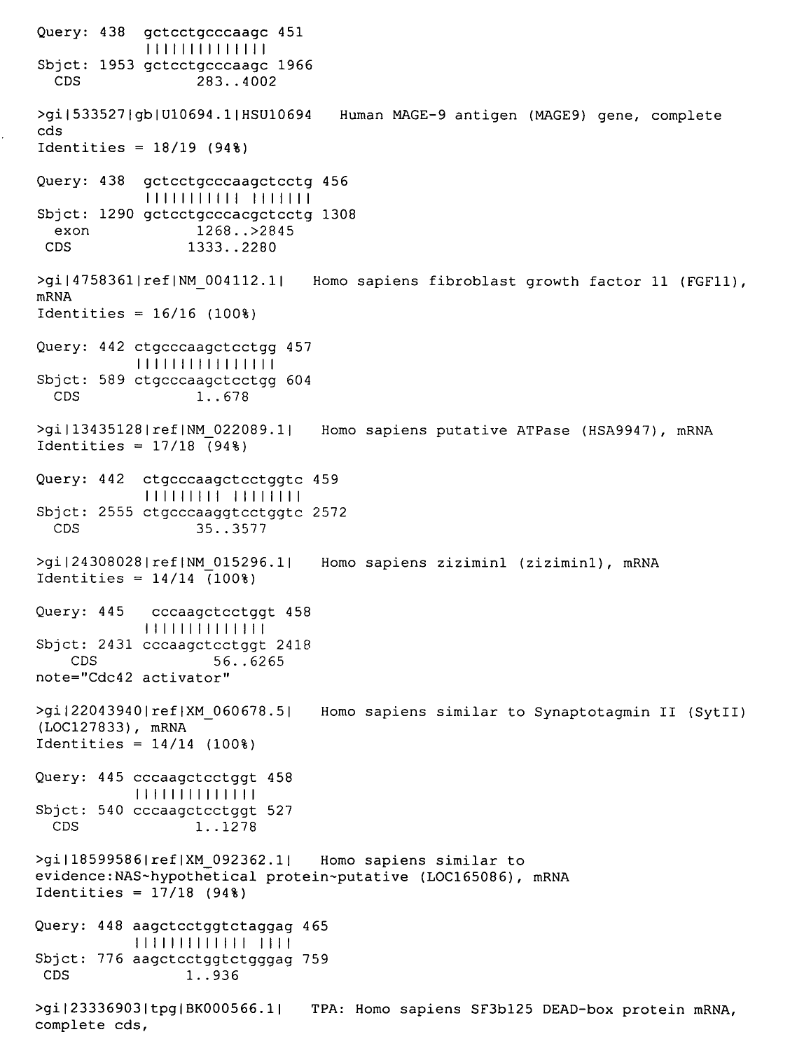

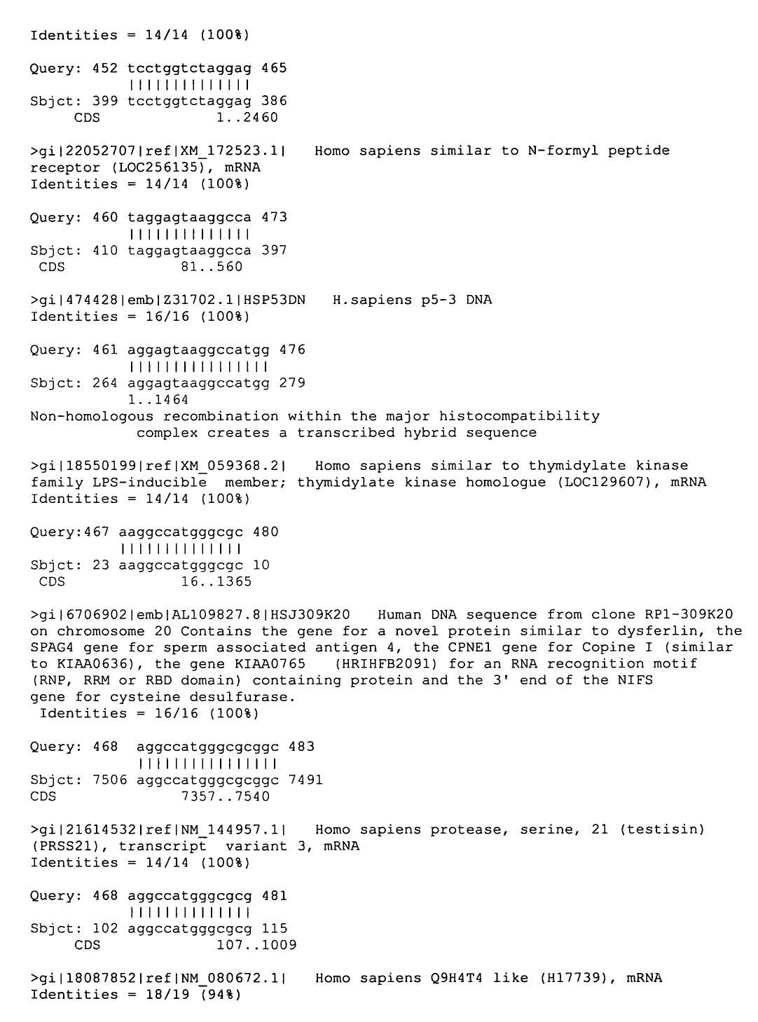

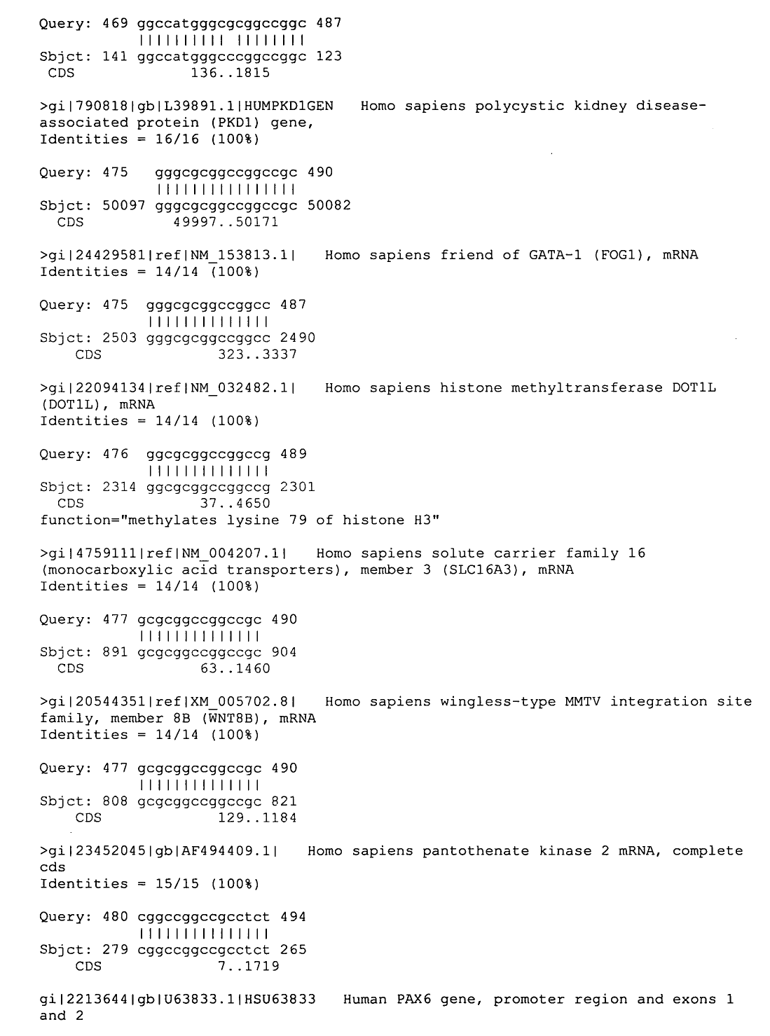

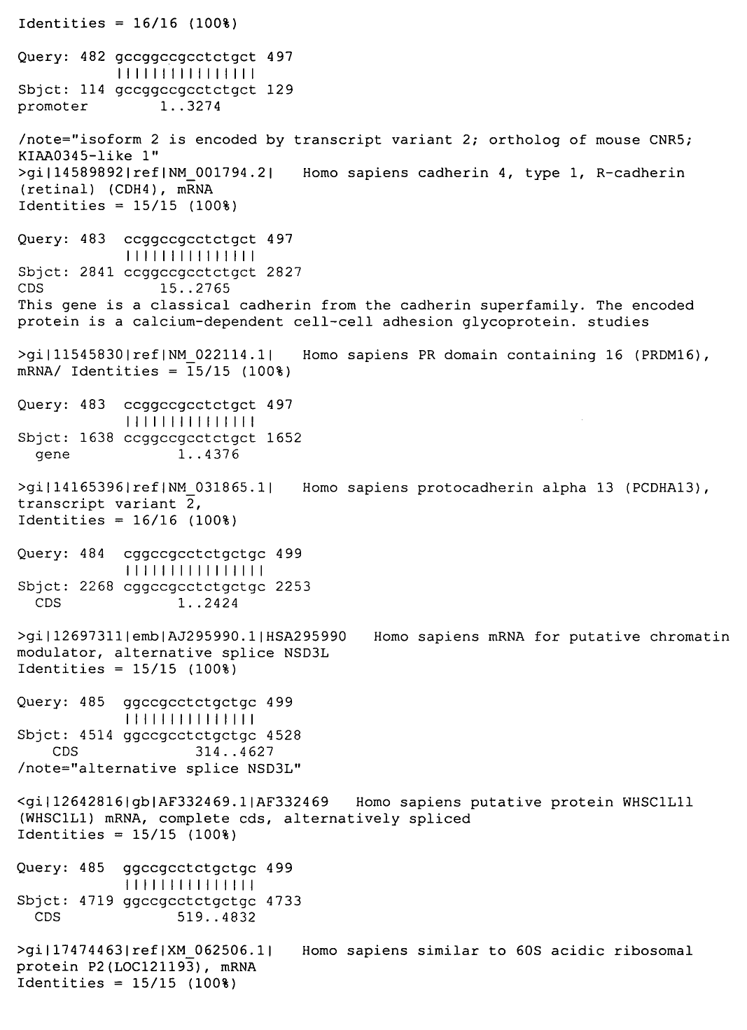

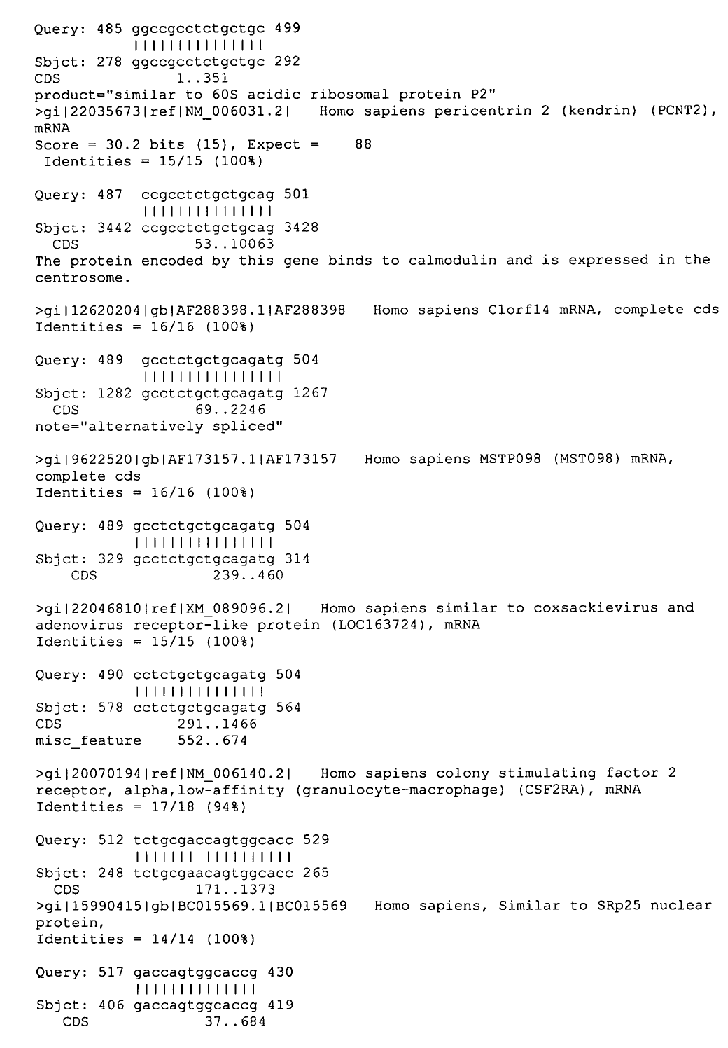

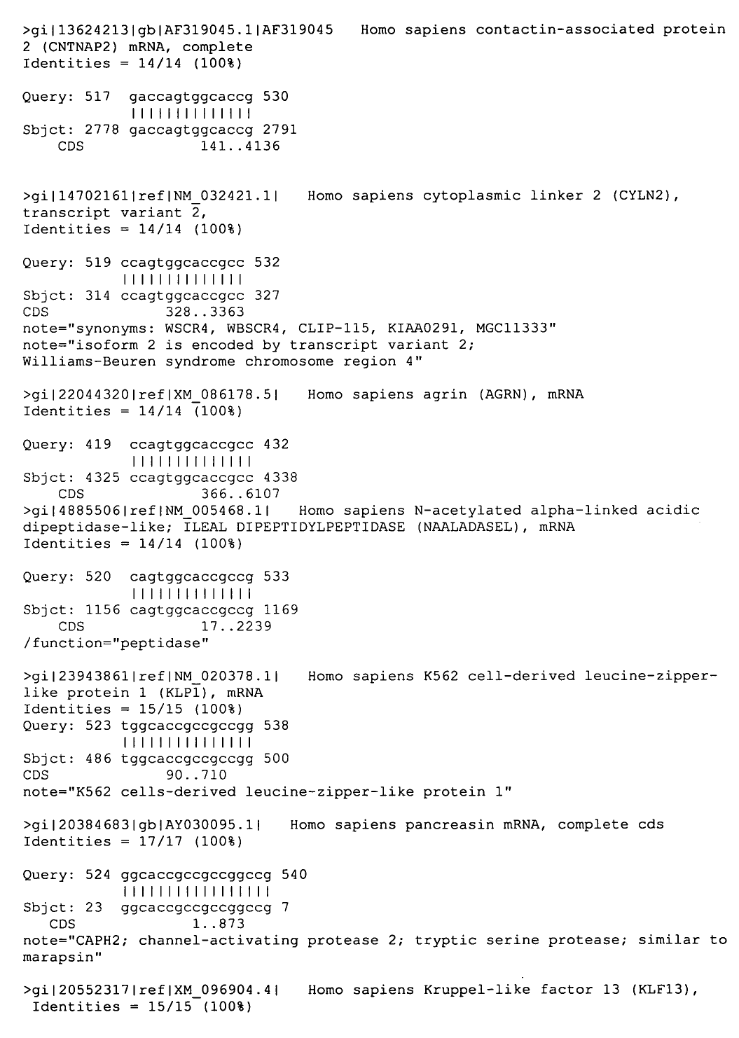

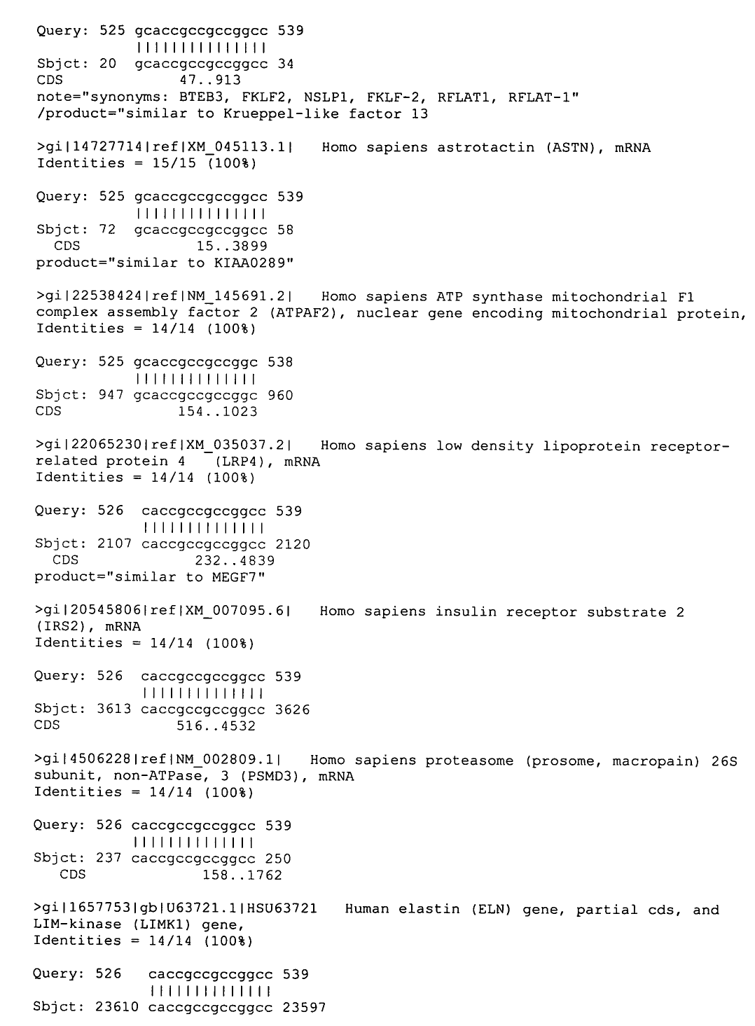

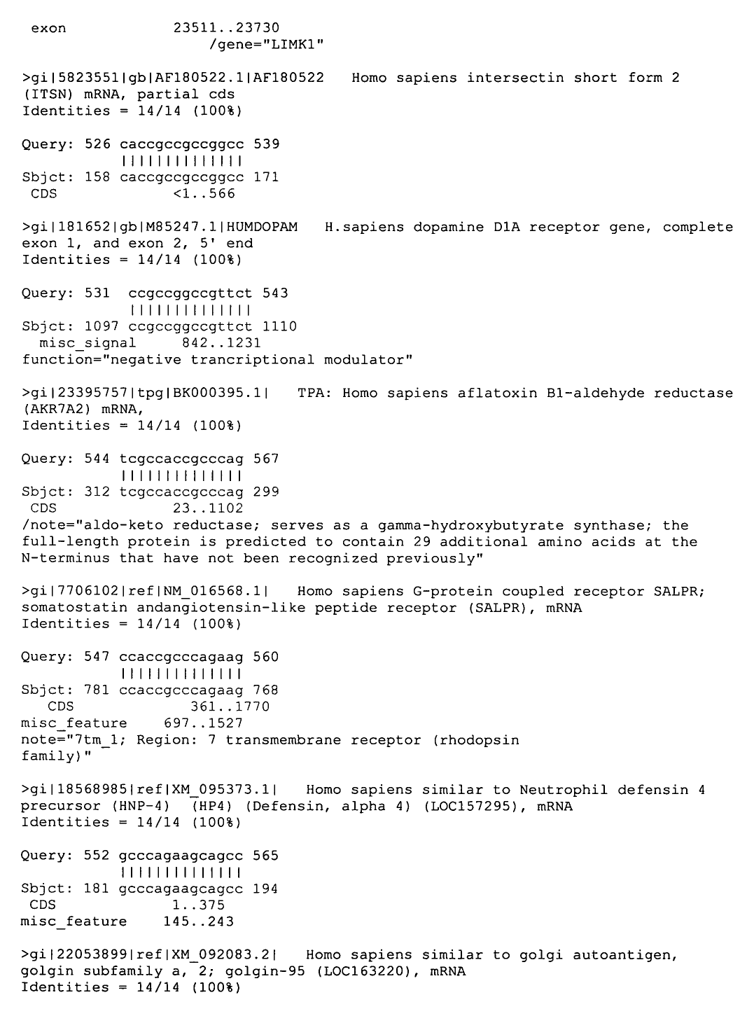

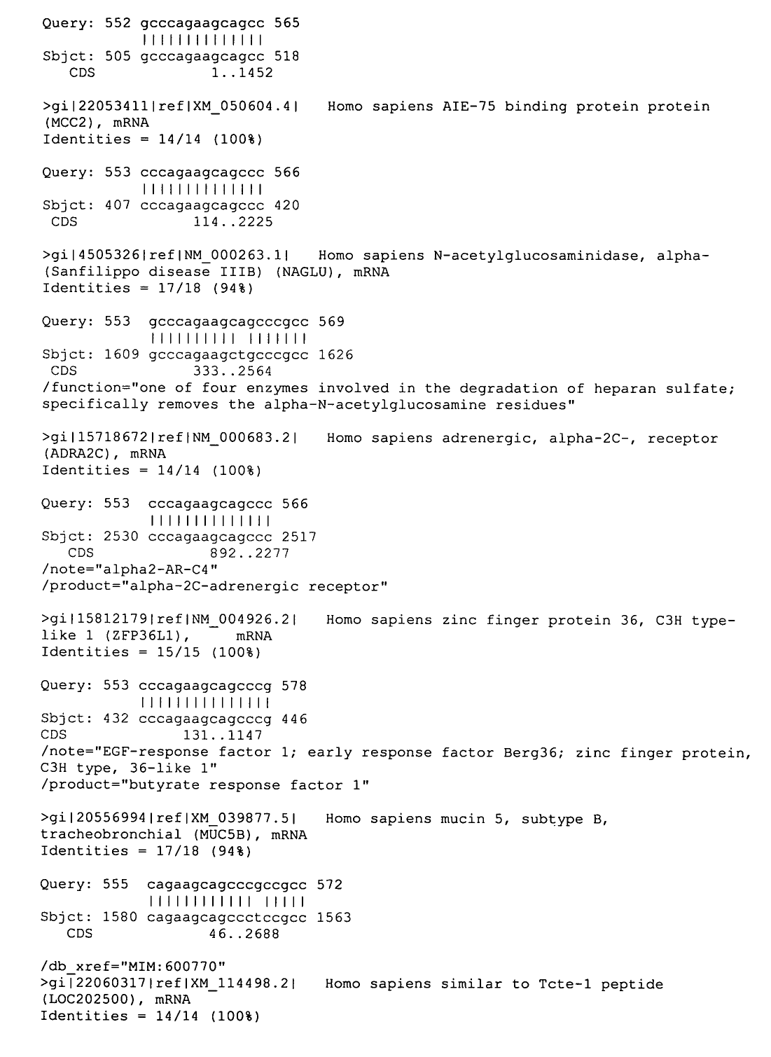

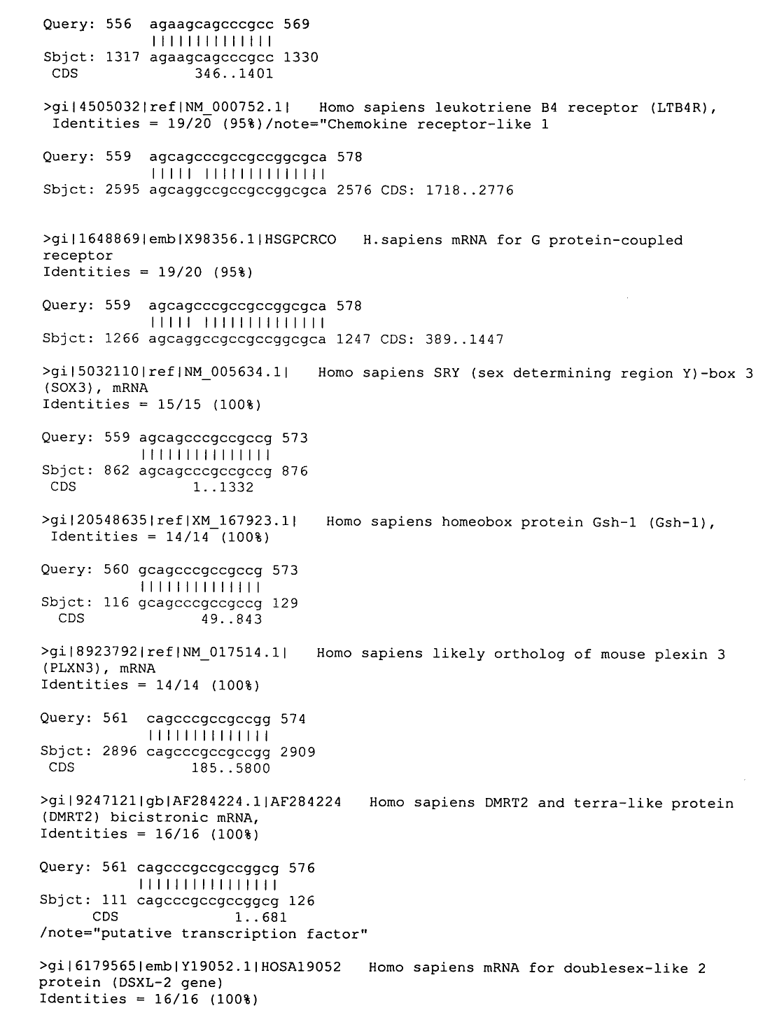

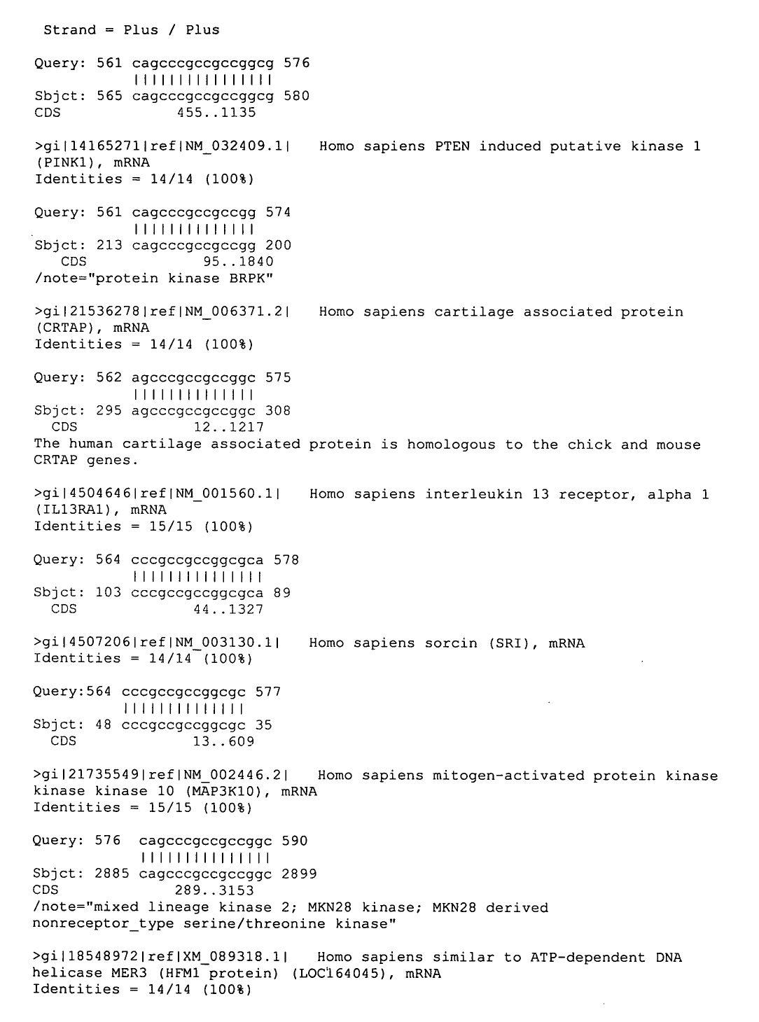

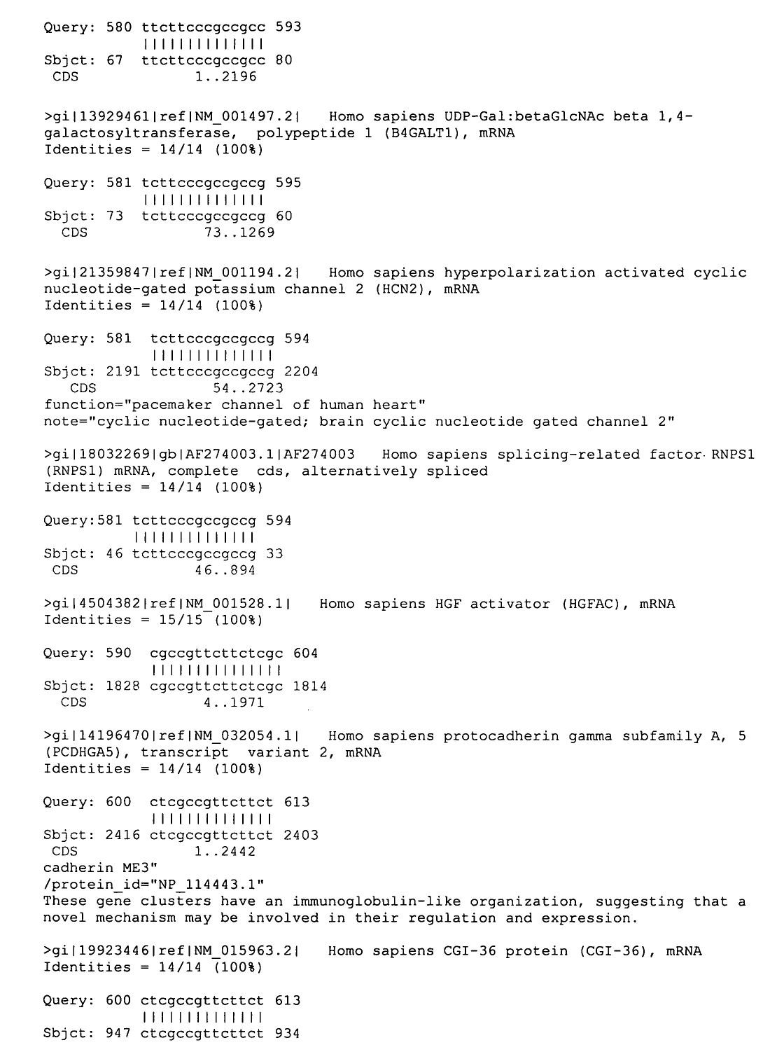

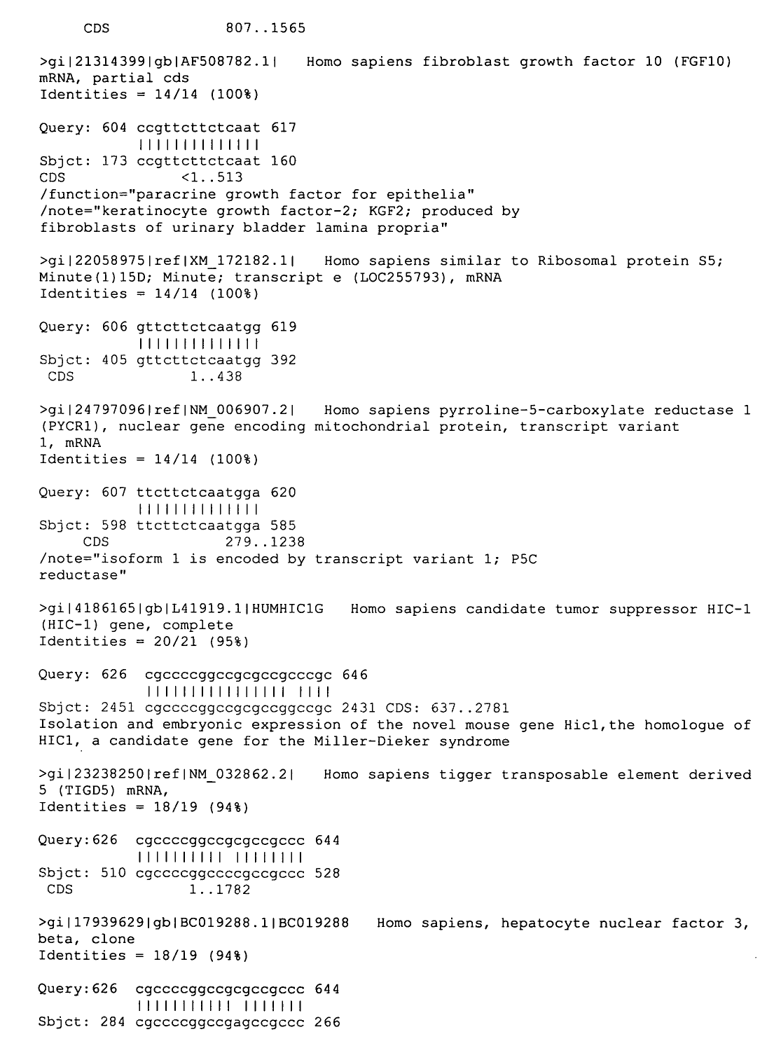

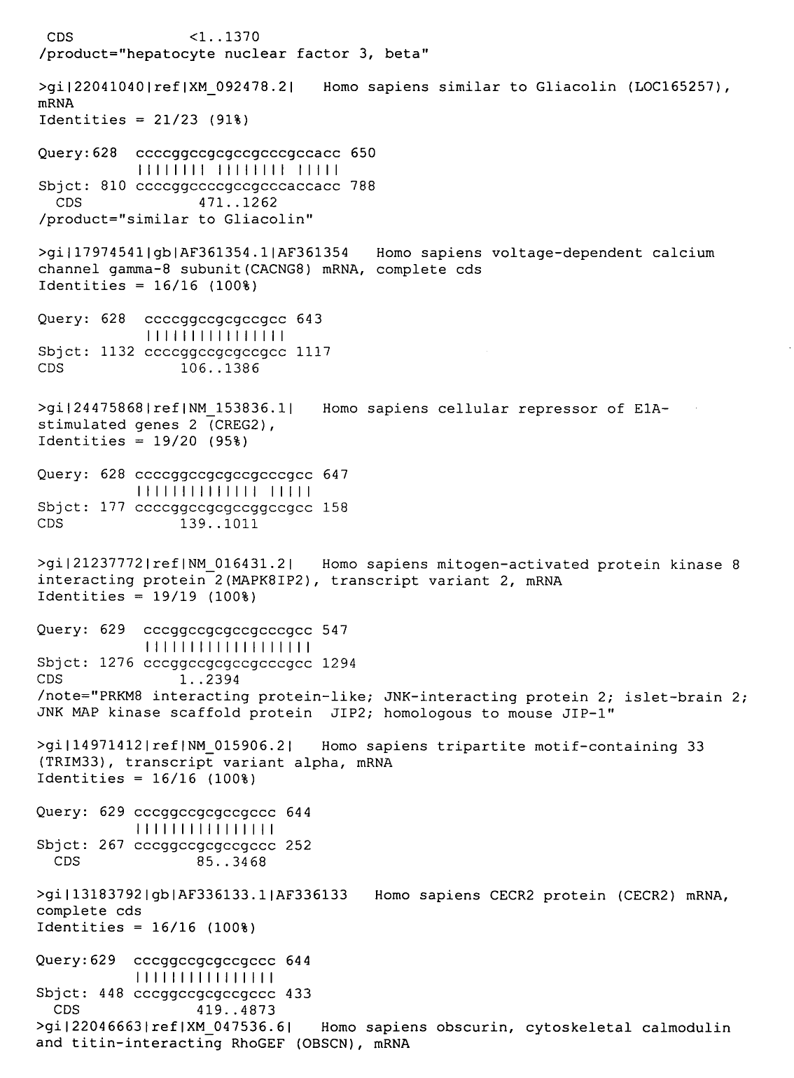

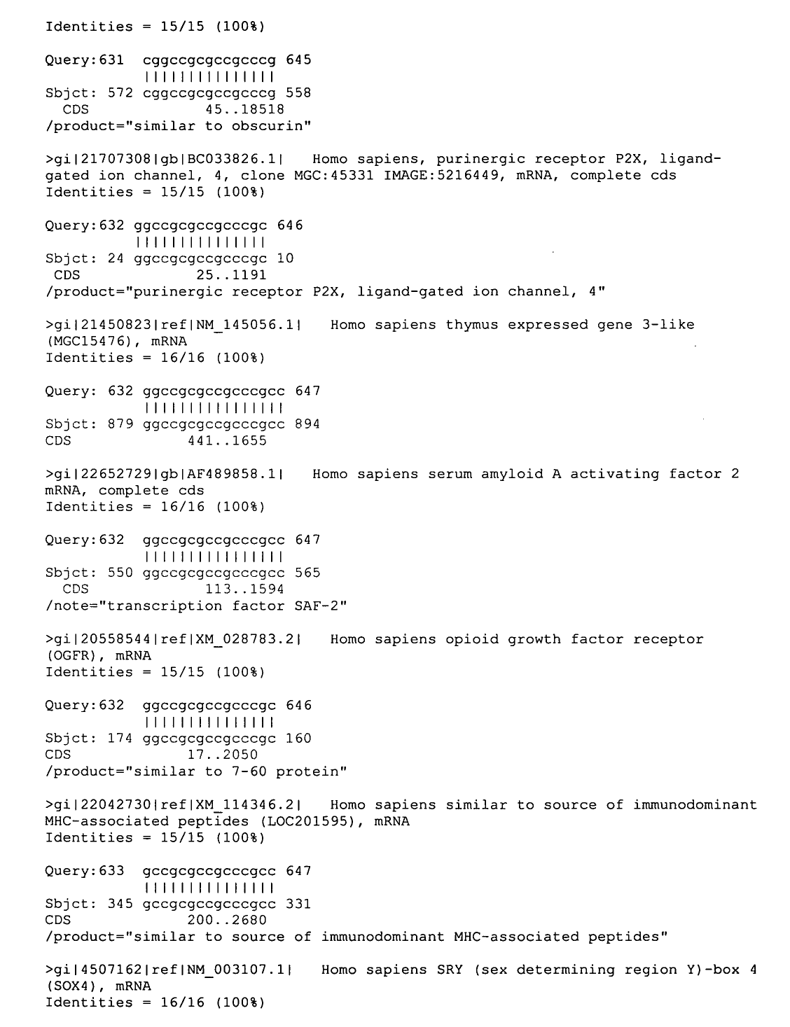

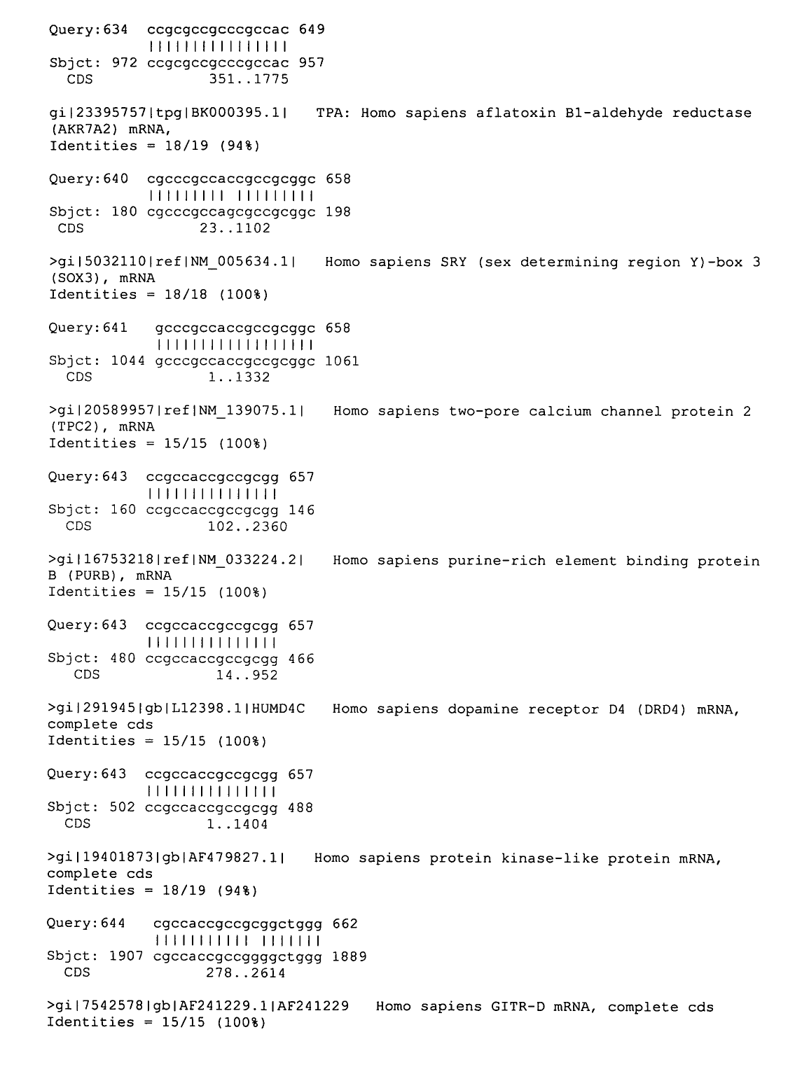

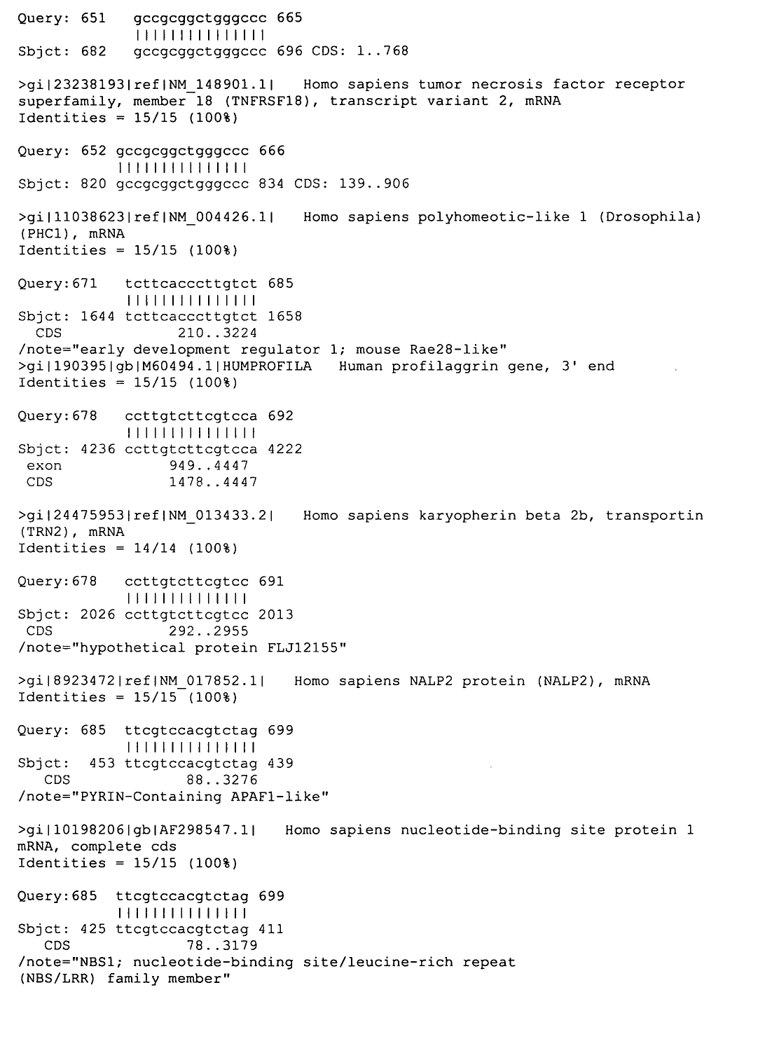

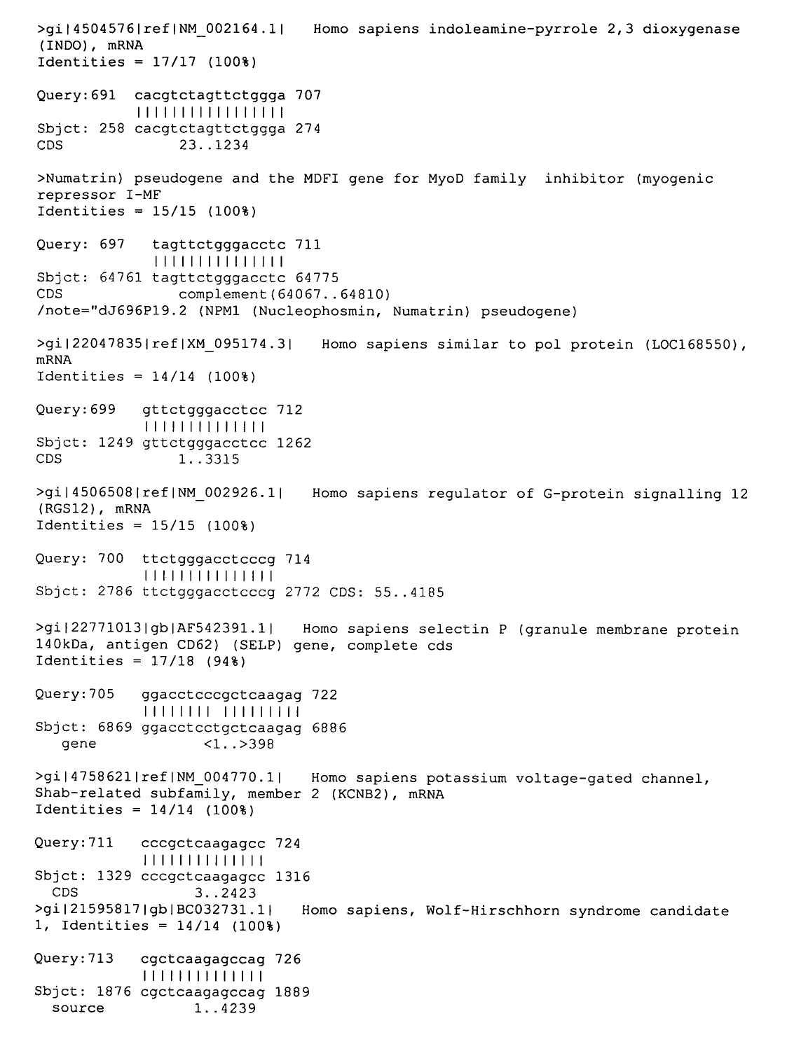

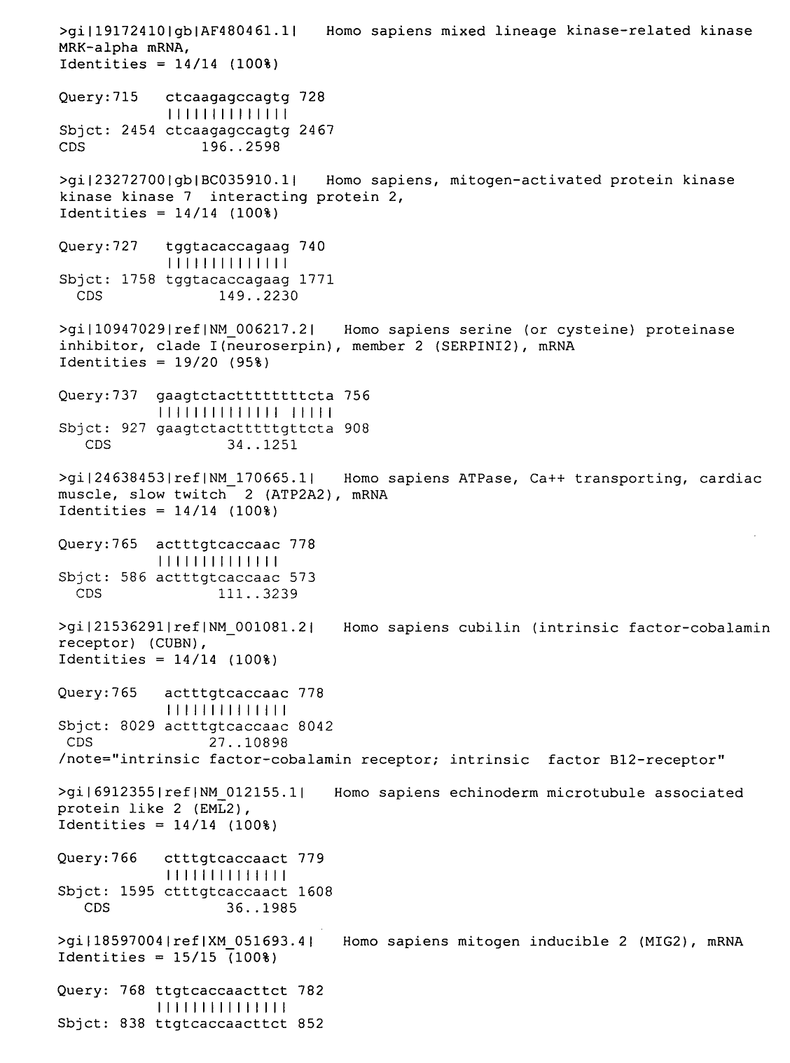

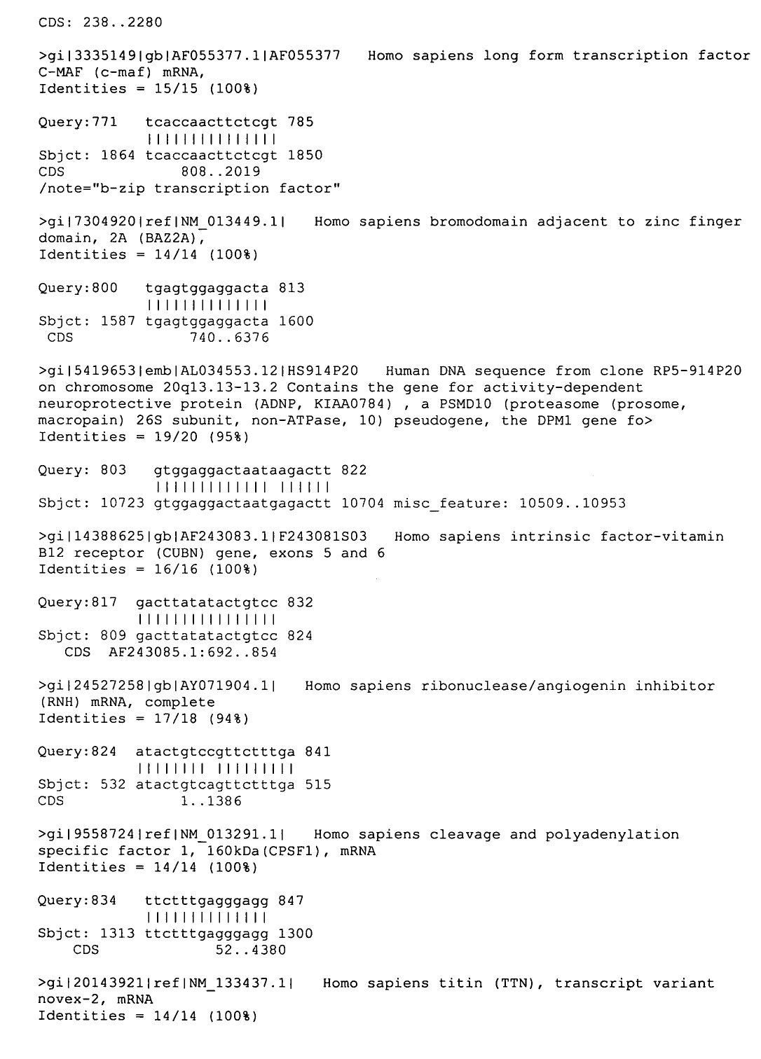

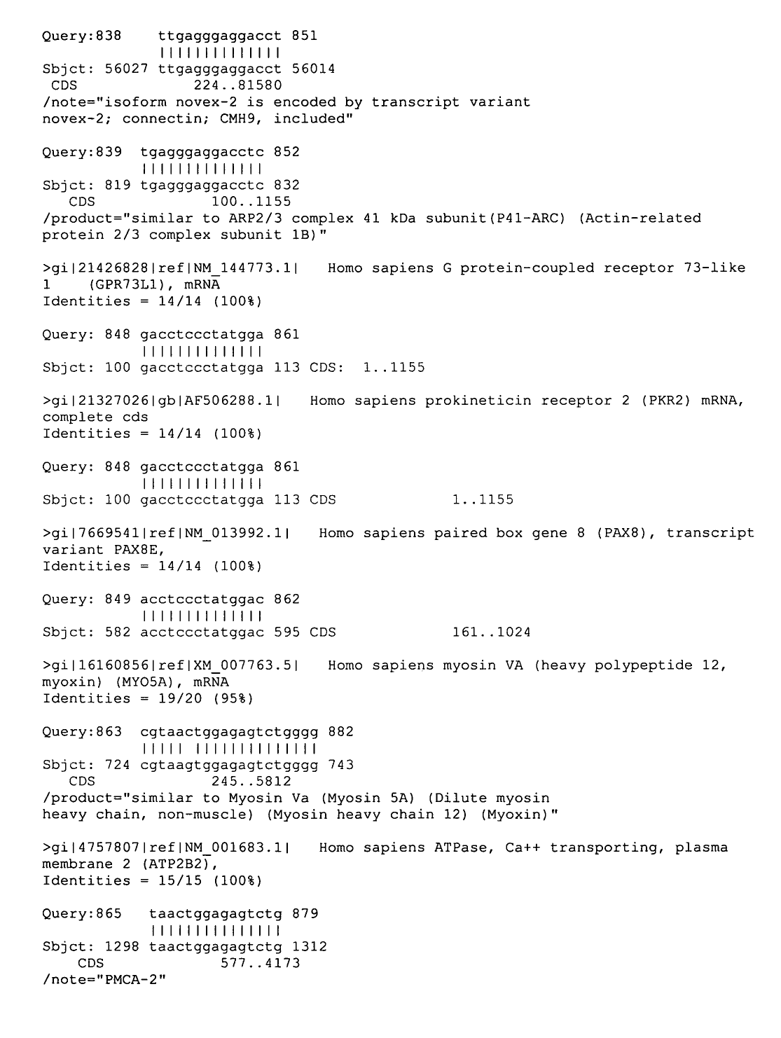

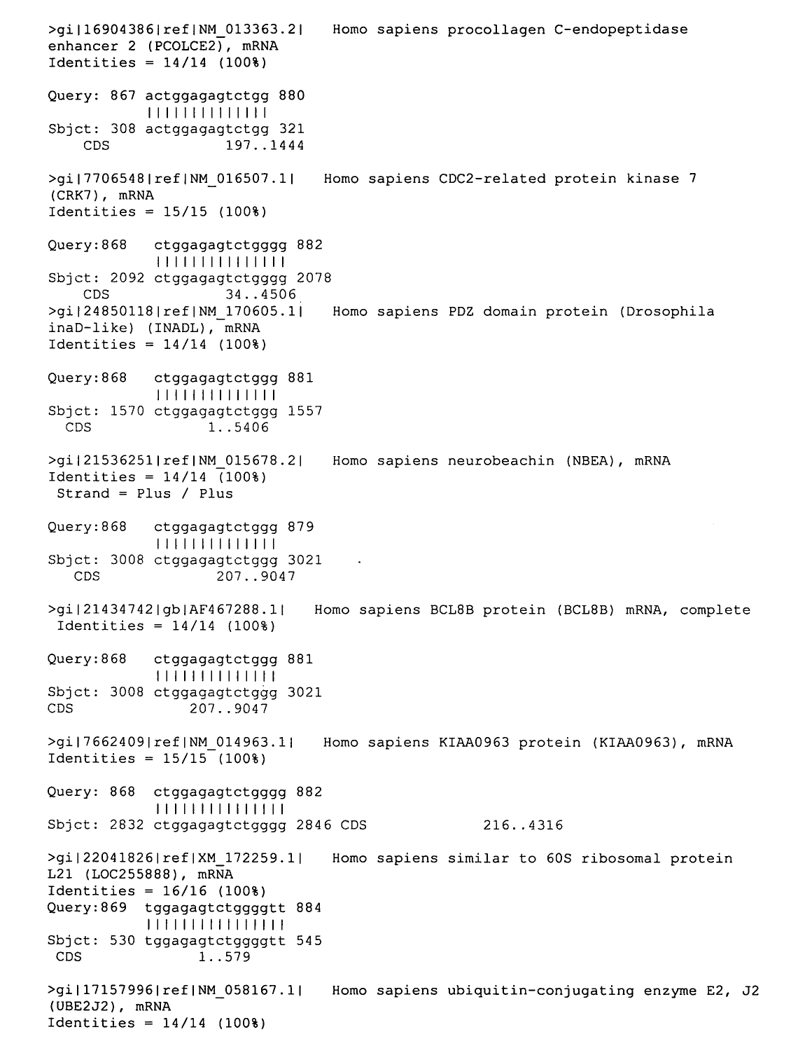

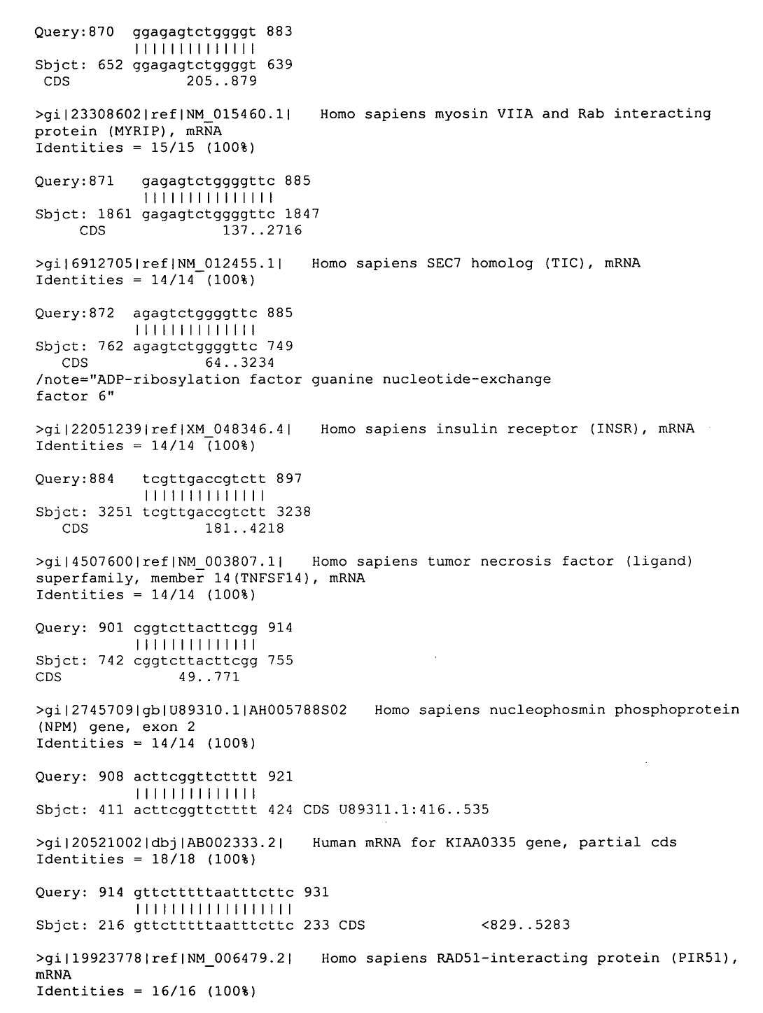

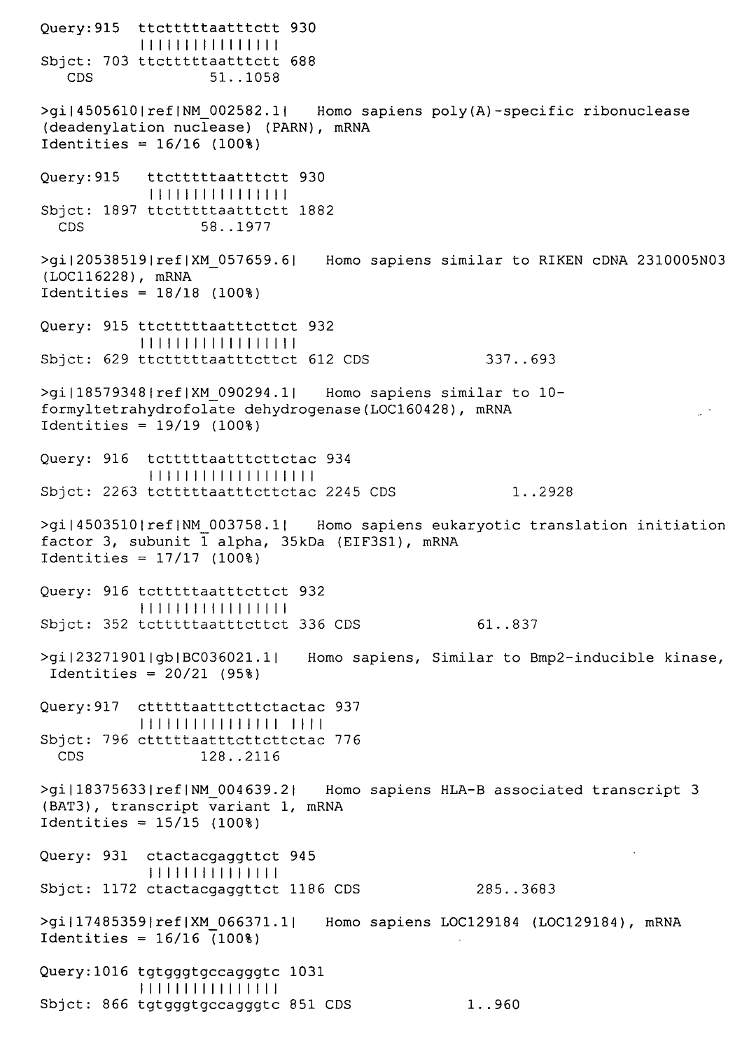

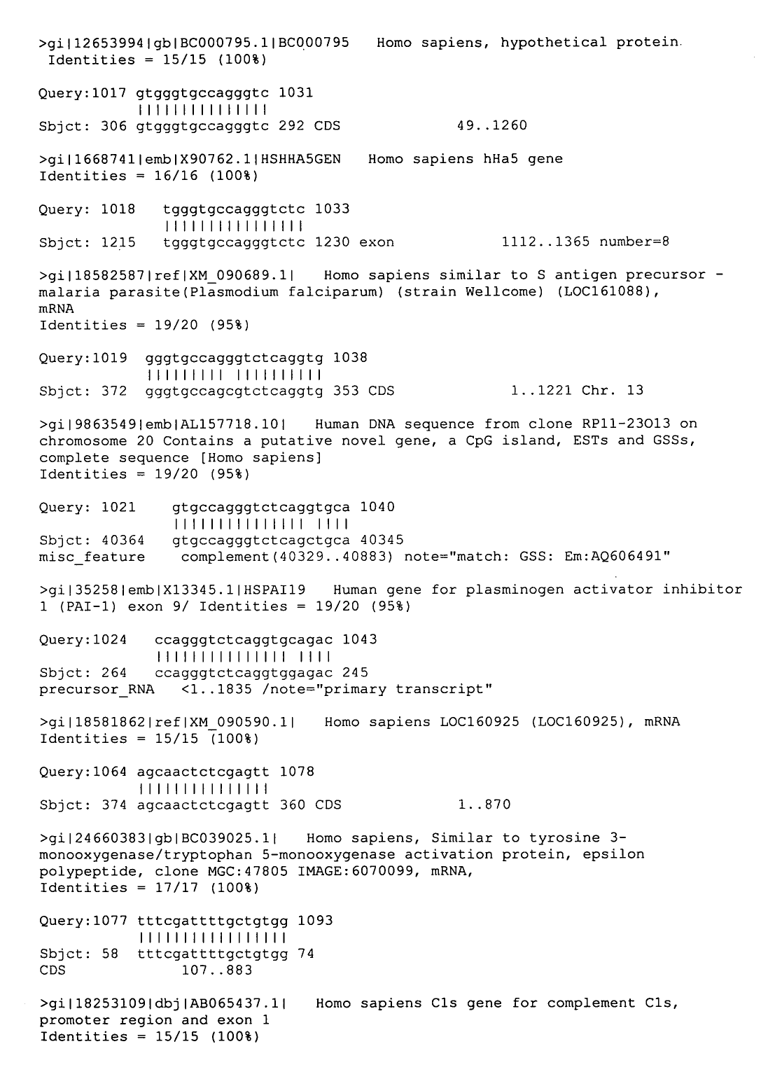

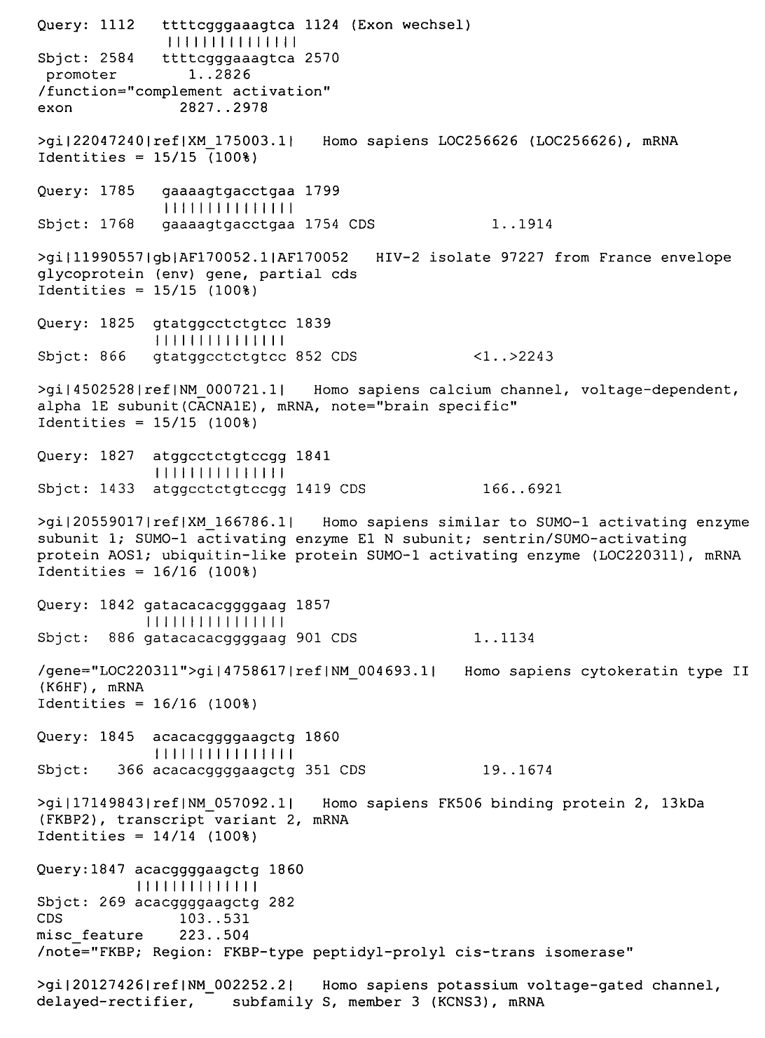

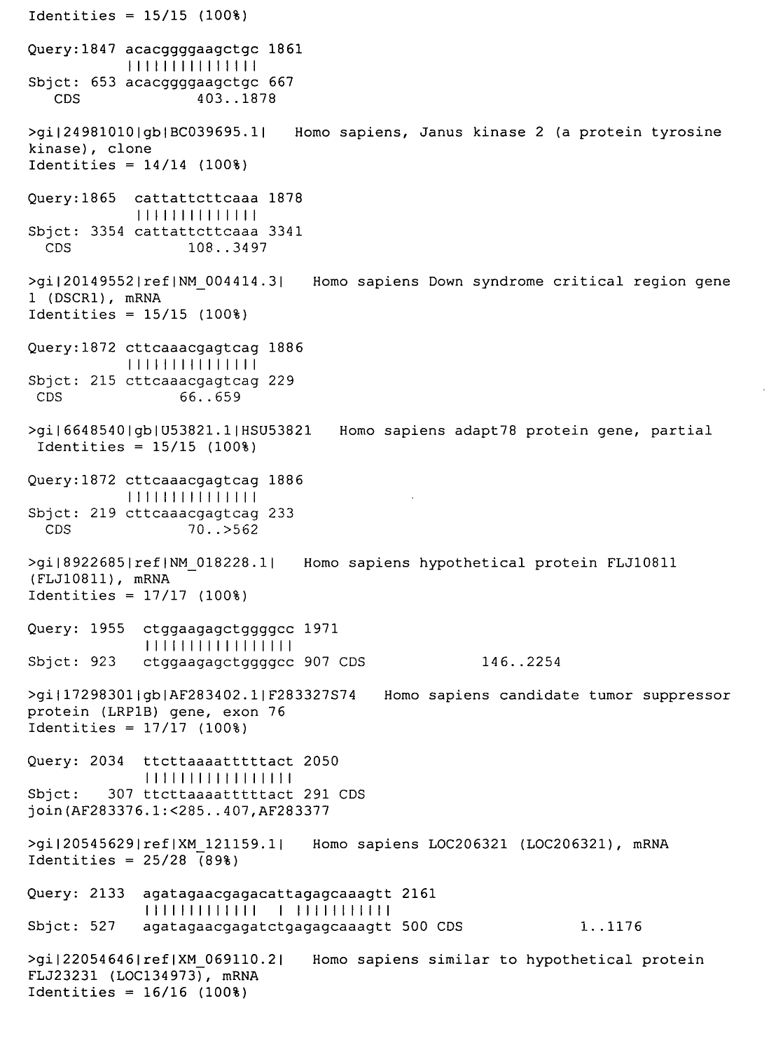

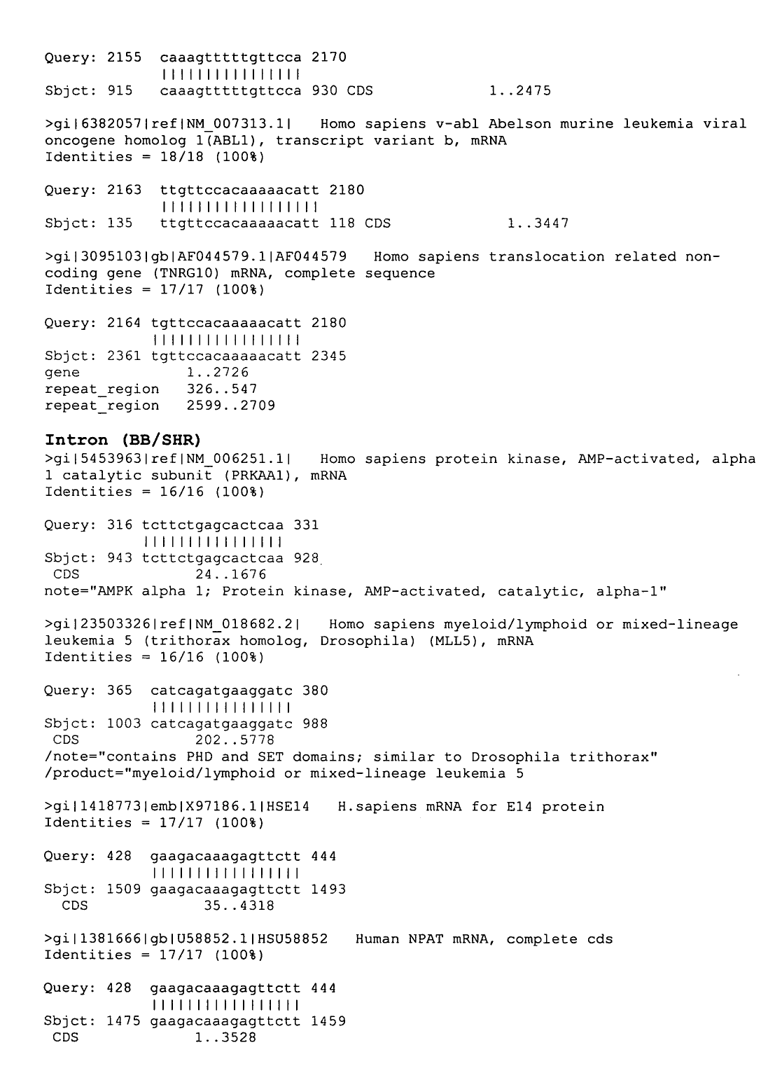

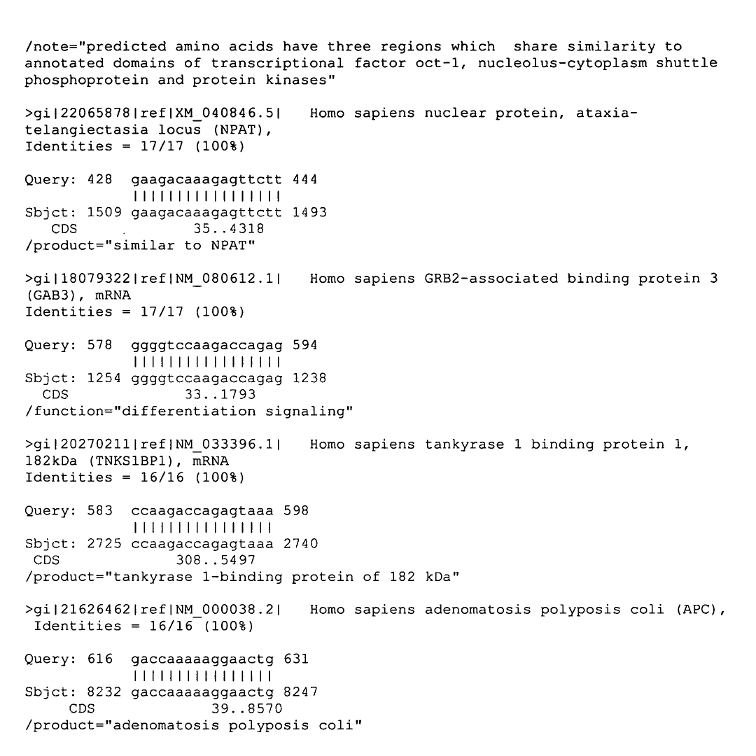

In view of the multifunctionality of YY1, antisense DNA of YY1 was checked against the sequences in the gene bank for match with other genes (see FIG. 13; the positions given (numberings) relate to the coding region and not to the nucleotide numbering of the Sequence Listing). These corresponding sequences must be applied in a targeted way, using nuclear localisation signals (NLS), with domains of YY1, to influence these genes (functional domains) (cf. FIG. 8).

The activation domains, repression domains and zinc fingers of YY1 must be created with the antisense DNA, both alone and in all combinatorial possibilities with each other. The combination depends on the disease condition, the organs involved and the sex.

Congenic and Transgenic Animals

As part of the present invention, in addition, congenic non-human mammals, preferably rats, were bred, which have a nucleic acid sequence that encodes (the mutated) YY1 based on SEQ ID NO:4. The mammal is preferably a rat. The mammals are characterised by decreased incidence of type 1 diabetes. In the same way, it is possible to breed transgenic animals, preferably rats, which are characterised by their having also a nucleic acid sequence that encodes (the mutated) YY1 (see above).

Moreover, the invention provides for the first time a method for the identification of diabetes protective agents, where the aforementioned mammals are administered potential active agents and a check is carried out as to what extent the tendency to develop type 1 diabetes is reduced. The subject of the invention is therefore also the use of a transgenic or congenic non-human mammal, whose germ and somatic cells contain a nucleic acid or a nucleic acid segment, which encodes a protein with the amino acid sequence shown in SEQ ID NO:4 (SHR) or an amino acid sequence homologous to that (see above), where the homologous amino acid sequence displays methionine at position 303 and arginine at position 311, for the purpose of identifying diabetes protective substances.

In addition, the invention relates to transgenic non-human mammals, in particular rats, whose germ and somatic cells contain a nucleic acid or a nucleic acid segment, which encodes a protein with the amino acid sequence shown in SEQ ID NO:2 or an amino acid sequence homologous to that (see above), where the homologous amino acid sequence displays arginine at position 303 and lysine at position 311.

Other Subjects of the Invention

The invention also relates to further subjects and embodiments that will be accessible without difficulty for the practitioner given the background of the present disclosure.

In this connection, devices (kits) for performing one of the aforementioned (screening) methods should also be mentioned.

Methods

1. Animal Welfare

2. Establishing Congenic BB.6S

2.1. Establishing Further Congenic Lines

3. Phenotypical Characterisation

3.1. Incidence of Diabetes and Determination of Blood Glucose

3.2. Lymphocyte Phenotypes

3.3 Blood Pressure

4. Statistical Analysis

5. Molecular Biology Methods

5.1. Isolation of the Islets of Langerhans of the Pancreas

5.2. DNA Isolation

5.3. RNA Isolation from Tissues

5.4. RT-PCR

5.5. YY1 Sequencing

5.6. Eluation of the Second Band on BB.6S

5.7. Gene Expression Studies

5.8. RNA Isolation from EDTA Blood

6. Fine Mapping of YY1 (ensuring that YY1 is in the diabetes protective region)

7. Checking Polymorphism in Intron 3 Using Other Rat Strains

8. Further In Vitro Investigations

9. Further In Vivo Investigations

10. Primer Conditions in Sequencing

11. Microsatellite Markers

1. Animal Welfare

The animals were kept under semi-barrier conditions and had free access to pelleted feed (Ssniff R-Zucht, Spezialitatën GmbH, Soest), as well as water (pH 2.5-3).

The animals were kept under a 12 hour rhythm (12 h light, 12 h darkness). There were no more than 3 animals in one cage.

All experiments involving the animals were performed in accordance with the animal protection regulations in force.

2. Establishing Congenic BB.6S

Male SHR/Mol rats that had been commercially acquired (Mollegaard Breeding Ltd, Denmark) were crossed with BB/OK females. The resultant F1 hybrids were back-crossed with diabetic BB/OK rats 7 times in all. The progeny of each back-cross generation were genetically analysed for heterozygoty in the chromosomal region of interest using PCR-analysed microsatellite markers that flank the regions of interest. Animals that were heterozygotic for this region were then mated to breed the next back-cross generation. Animals of the 7^{th} back-cross generation that were heterozygotic for the chromosomal region were then interbred (intercross) and again genetically analysed. First of all, all animals that were homozygotic for allele of the SHR rats for the chromosomal region of interest were selected. These animals were then again genetically characterised using 139 PCR analysed microsatellite markers covering about 96% of the rat genome. Animals that were then homozygotic for allele of the BB/OK rat for these 139 markers were then used to establish (founder) the congenic BB.SHR lines and characterised phenotypically.

The marker spectrum was broadened, so as, on the one hand, to secure the genetic BB/OK background (See Material & Methods section), and, on the other, to be able to recognise any recombinations that might occur in small regions: D6Rat3-D6Rat1-D6wox13-D6Rat101-Ighe/Ckb-D6Mgh2-D6Rat94-D6Rat183-D6Rat10-D6Rat7-D6Rat75-D6Rat9-D6Rat6-D6Rat160-D6Mgh9-D6Rat184.

These lines were used for further breeding with the intention of minimising the diabetes protective region. By establishing various subcongenic lines (BB.6Sa-f) with the phenotype of the BB.6S rat, the relevant chromosomal region was narrowed down to <2 cM (30-40 genes).

Mapping of the diabetes protective genes around the locus D6Mgh2.

TABLE 2

| cM | | BB.6S | BB.6a | BB.6b | BB.6c | BB.6d | BB.6e | BB.6f |
|---|---|---|---|---|---|---|---|---|
| | D6Mgh4 | BB | BB | BB | BB | BB | BB | BB |
| 2.1 | D6Rat13 | BB | BB | BB | BB | BB | BB | BB |
| 4.4 | D6Wox5 | BB | BB | BB | BB | BB | BB | BB |
| 1.5 | D6Rat66 | BB | BB | BB | BB | BB | BB | BB |
| 2.0 | D6Rat184/D6Mgh9 | SHR | BB | BB | BB | BB | BB | SHR |
| 0.8 | D6Rat160 | SHR | BB | BB | BB | BB | BB | SHR |
| 1.3 | D6Rat6 | SHR | BB | BB | BB | BB | BB | SHR |
| | D6Rat9 | SHR | BB | BB | BB | BB | BB | SHR |
| 0.4 | D6Rat75 | SHR | BB | BB | BB | BB | BB | |
| 0.4 | D6Rat7 | SHR | BB | BB | BB | BB | BB | SHR |
| 2.1 | D6Rat10 | SHR | BB | BB | BB | BB | BB | SHR |
| 1.6 | D6Rat183 | SHR | BB | SHR | BB | BB | BB | SHR |
| 0.4 | D6Rat94 | SHR | BB | SHR | BB | BB | BB | SHR |
| 1.9 | D6Mgh2 | SHR | SHR | SHR | BB | BB | BB | SHR |
| 1.7 | Ighe | SHR | SHR | BB | SHR | SHR | SHR | SHR |
| | Ckb/ | SHR | SHR | BB | SHR | SHR | BB | SHR |
| | D6Rat101 | | | | | | | |
| 1.0 | D6Rat1 | SHR | SHR | BB | BB | BB | SHR | BB |
| 1.9 | D6Rat3 | SHR | SHR | BB | BB | SHR | SHR | BB |
| | Incidence | 15% | 10% | 22% | >50% | >50% | >50% | 14% |
| | of | | | | | | | |
| | diabetes | | | | | | | |

2.1. Establishing Further Congenic Lines BB.K(arlsburg)W(ild)R(at) Rats

Along with diabetes resistant SHR rats, wild rats were also used for breeding congenic BB.K(arlsburg)W(ild)R(at)rats. Analogously to the BB.6S line, the same chromosomal region of the BB/OK of 15 cM was exchanged with that of wild rats (BB.6W).

See Establishing Congenic Rat Lines under Point 2.

3. Phenotypical Characterisation

Tye body mass of non-diabetic rats was measured in the twelfth week. (NAGEMA scales, VEB Wägetechnik Rapido, weight range 1-1000 g, e=0.1 g).

In addition, at this time, blood glucose, serum insulin, plasma cholesterol and triglyceride were measured. Blood samples were performed by puncturing the eye plexus under isoflurane anaesthesia (Abbott, Wiesbaden, Deutschland).

The blood fats were passed through automated analysis (Roche Cobas Mira Plus, Roche, Switzerland). The ELISA method (Rat Insulin ELISA, Mercodida AB, Uppsala, Sweden) was used to determine serum insulin concentration.

3.1. Incidence of Diabetes and Determination of Blood Glucose

The incidence of diabetes of the strains was determined longitudinally in one inbred generation. In addition, from the 50^{th} to the 200^{th} day of life, testing for glucosuria was performed twice a week using a test strip (Diaabur-Test 5000, Boehringer, Mannheim, Germany) and if the result was positive, blood sugar was measured (Analyzer ESAT 6660-2, Prüfgerätewerk Medingen GmbH). If within a 2 day period hyperglycaemia (blood glucose >15 mmol/l corresponds to >300 mg/dl) was identified, the rats were held to be diabetic.

3.2. Lymphocyte Phenotype

Lymphocyte isolation of non-diabetic rats was obtained by density gradient centrifugation using Histopaque 1083 (Sigma-Aldrich, Deisenhofen, Deutschland).

Analysis was by two-colour immunofluorescence technique. (EPICS Profile II Flow Cytometer (Coulter, Hialeah, USA).

A minimum number of 0.5×106 cells was specified for phenotyping. The following monoclonal antibodies were used:

| Cells | MABs | Company |
|---|---|---|
| T-cells | R73-FITC | Medizinische Hochschule, Hannover, |
| | | Germany |
| T-cells | 3G2-FITC | Medizinische Hochschule, Hannover, |
| | | Germany |
| B-cells | OX33-FITC | Pharmigen, Heidelberg, Germany |
| NK-cells | 10/78-FITC | Pharmigen, Heidelberg, Germany |
| TH-cells | W3/25-FITC | Serotec, Eching, Germany |
| Ts/c-cells | 341-FITC | Pharmigen, Heidelberg, Germany |
| Act.T-cells | OX39-PE | Pharmigen, Heidelberg, Germany |
| TH1/TH2-like | OX-22-PE | Pharmigen, Heidelberg, Germany |
| cells | W3/25-FITC | Serotec, Eching, Germany |

3.3. Blood Pressure

Systolic blood pressure was measured at the age of 12 to 14 weeks between 09:00 and 11:00 am using tail-cuff measurement (Kent Scientific Corporation, Kent, England). Three separate points were used to obtain a final value.

4. Statistical Analysis

Calculation of the means and standard deviation (SD) was carried out using SPSS statistics software. The significances were calculated using ANOVA analysis.

5. Molecular Biology Methods

5.1. Isolation of the Islets of Langerhans of the Pancreas

Isolation of the islets of Langerhans was performed using collagenase digestion (Biochrom) and dextran density gradient centrifugation (Dextran, Fluka, Switzerland). At least 3 and no more than 15 pancreatas were used per collagenase digestion.

After removal of the pancreata, these were transferred to the collagenase solution (30 mg collagenase (Biochrom) dissolved in 40 ml Hank's balanced salt solution (Sigma) at 37° C. for 10 min) and digested using hand shaking. Next, the islet sediment was washed three times in Hank's balanced salt solution. After centrifugation of the islets (1900 rpm), the residue was decanted and agitated with dextran solution (11 g dextran/39 ml Hank's balanced salt solution). The islet emulsion was then coated with 3 more density gradients each of 4 ml. After centrifugation lasting 20 minutes (1900 rpm), selection of the islets was performed by hand under the microscope. For any given gene expression analysis, 500 islets of Langerhans were used each.

5.2. DNA Isolation

Isolation of genomic DNA from liver tissue was performed according to the manufacturer's instructions (Wizard®, Genomic DNA Purification Kit, Promega).

5.3. RNA Isolation From Tissues

RNA isolation was performed in accordance with the specifications of the manufacturer (RnEasy Mini Kit, Qiagen).

5.4. RT-PCR

Transcription of the RNA into ssDNA (cDNA) was performed using the Reverse Transcription System from Promega.

The RNA concentration (Photometer, Eppendorf) was set at 1.5 μg for transcription.

The 1.5 μg RNA were mixed with 1 μl random hexamer primer (Promega) and then filled with Rnase-free water (Promega) to obtain a 13.5 μl final volume. This RNA mix was subsequently heated for 5 min at 65° C. and then cooled on ice for one minute. After adding the RT (reverse transcriptase) mix (6.5 μl) transcription took place in the cycler (Thermocycler, Techne, Cambridge) using the following program:

| | |
|---|---|
| 50 min | 42° C. |
| 10 min | 70° C. |

RT mix contains:

| Volume | | Components | Company |
|---|---|---|---|
| +4 | μl | 5x MMLV RT buffer | Promega |
| +1 | μl | 10 mM dNTPs | Promega |
| +0.5 | μl | rRNasin | Promega |
| +1 | μl | MMLV reverse | Promega |
| | | transcriptase | |
| 6.5 | μl | Final volume | |

After completion of the cycler program the ssDNA was mixed with 50 μl bidest. water.

The ssDNA was used for the expression studies and for sequencing.

5.5. YY1 Sequencing

For sequencing of the gene, genomic DNA from liver tissue (Wizard®, Genomic DNA Purification Kit, Promega) and RNA from isolated islets of Langerhans, pancreas, liver and brain were extracted from the BB/OK, SHR and BB.6S rat (Rneasy Mini Kit, Qiagen). RNA was then transcribed into ssDNA and/or cDNA (see Point 5.4) according to the manufacturer's specifications (Reverse Transcription System, Promega). Both genomic and cDNA were so amplified using PCR (using GeneAmp® High Fidelity PCR System, Applied BioBiosystems) and various primers, that overlapping gene products for sequencing became available (cf. FIG. 11; promotor: K823-F/K825-R*; promotor and exon 1: K884-F/K806-R; exon 1: K801-F/K804-R; K814-F/KK832-R*; exon 2 and 3: K828-F/K833-R; K831-F/K817-R; exon 4: K815-F/K870-R; K815-F/K818-R; K816-F/K819-R; K834-F/K836-R). PCR was performed as follows: 3 min. at 94° C., followed by 35 cycles each of 30 sec at 94° C., 1 min. at 55 or 60° C. (*), 45 sec. at 72° C. with subsequent elongation of 3 min. at 72 ° C.

After purification with Amicon®Microcon PCR centrifugal filters (Millipore, USA), the amplified gene products were used for the sequencer PCR. The purified PCR products (250 ng) were amplified using ABIPRISM® BigDye Terminators v3.0 Cycle Sequencing Kit in line with the manufacturer's instructions (Applied Biosystems) (25 cycles: 10 sec. at 96° C., 4 min. at 60° C.). The amplified gene products were then precipitated with alcohol, dried, absorbed in formamide (4 μl) and Loading Buffer (1 μl), denatured for 2 min at 90° C., and then sequenced with the ABI PRISM® 377 (Applied Biosystems). Sequence analysis, including generation of the protein sequence, was carried out using the sequence analysis system software v3.4.1 (Applied Biosystems, USA). The gene products were repeatedly sequenced and analysed to exclude artefacts.

Checking of the sequence with the gene bank was done online (www.ncbi.nlm.nih.gov/BLAST).

5.6. Elution of the Second Band on BB.6S

After gelelectrophoretic separation, the second band (pancreas, liver, brain) was cut out and eluted with the Wizard®PCR Preps DNA Purification System from Promega. After amplification with the primers K831-F/K870-R, sequencing was carried out on the ABI PRISM® 377 (Applied BioBiosystems (cf. FIG. 12 and SEQ ID NO:7).

5.7. Gene Expression Studies

Transcription into ssDNA was performed from extracted RNA of isolated islets of Langerhans, pancreas, liver, brain, thymus and spleen of 8 day old rats of the inbred strains BB/OK, SHR, BB.6S and LEW (“neutral” control) (see Point 5.4). The optimum PCR conditions were developed for each primer pair using ssDNA diabetes resistant LEW rats. GAPDH gene expression was used as the basis for the control situation (housekeeping gene). Tissue-specific cDNA was amplified with the primers for GAPDH (F: 5′TCCCTCAAGATTGTCAGCAA3′; R: 5′AGATCCACAAACGGATACATT3′, product size=308 bp) and the YY1 primers K801-F/KK804-R (exon 1) and K831-F/K8818-R and/or K831-F/K870-R (zinc finger). The gene products were eletrophoretically separated and the expression strength quantified between the strains allowing for GAPDH and using the Typhoon 8600 Variable Mode Imager (Amersham-Pharmacia Biotech, U.K.).

5.8 RNA Isolation From EDTA Blood

| Product: | Company |
|---|---|
| QIAamp ® RNA Blood Mini Kit | QIAGEN |

Additional use of:

| Product: | Company |
|---|---|
| QIAshredder | QIAGEN |
| RQ1-RNase free DNase | Promega |

Full instructions etc. in the QIAGEN Mini Handbook supplied

6. Fine Mapping of YY1 (Ensuring that YY1 is in the Diabetes Protective Region)

Fine mapping was performed with the aid of the mouse gene sequence. Using this gene sequence, primers could be established.

7. Checking Polymorphism in Intron 3 Using Other Rat Strains

For this the following strains, kept under the same conditions as BB.6S, were used: 2 wild animals (wild type, M+F), 1 animal of each of diabetes resistant, inbred DA/K, BN/Mol, LEW/K and WOKW rats.

8. Further In Vitro Investigations

Morphological examinations of the pancreas of normoglycaemic BB/OK and BB.6S rats (Lucke, S., Klöting, I., Pusch, A., Heinrich, H. W. and H. J. Hahn. Endocrine pancreas histology of congenic BB-rat strains with reduced diabetes incidence after genetic manipulation on chromosomes 4, 6 and X. Autoimmunity 36, 2003, 143-149).

Examinations of isolated islets of Langerhans of BB/OK and BB.6S rats using variation in Ca concentration in the culture medium.

Calbindin-D28k protein expression using Western blot analysis under basal conditions and with increasing calcium concentration in the medium.

9. Further In Vivo Investigations

Studies on fracture healing in BB/OK and diabetes resistant LEW.1A rats (38).

BB/OK and BB.6S rats were fed a standard diet (0.8% Ca), a high Ca (2%) and a low Ca diet (0.4%) (produced by Ssniff R-Zucht, Spezialitäten GmbH, Soest) and observed for development of diabetes during their first 30 weeks of life (unpublished).

Administration of Ca channel blockers or various Ca antagonists (Quercitin). (unpublished)

10. Primer Conditions in Sequencing

The following conditions apply for all primers:

Denaturation: 3 min at 94° C.

Annealing: 35 cycles each of 30 sec at 94° C.
1 min at 55 or 60° C. (*)
45 sec at 72° C.

Elongation: 3 min at 72° C.

11. Microsatellite Markers

| Chr. | Internal | Marker | Total |
|---|---|---|---|
| 1 | R152 | Igf2 | |
| 1 | K6 | D1Mgh10 | |
| 1 | K7 | D1Mgh12 | |
| 1 | K57 | D1Mgh7 | |
| 1 | K58 | D1Mit3 | |
| 1 | K109 | D1Mgh6 | |
| 1 | K136 | D1Mgh4 | |
| 1 | K220 | D1Mgh18 | |
| 1 | K228 | D1Mgh14 | |
| 1 | K524 | D1Rat249 | |
| 1 | K526 | D1Rat167 | |
| 1 | K533 | D1Rat60 | |
| | | | 12 |
| 2 | K110 | D2Mit8 | |
| 2 | K111 | D2Mgh9 | |
| 2 | K138 | D2Mit1 | |
| 2 | K139 | D2Mit4 | |
| 2 | K140 | D2Mit15 | |
| 2 | K230 | D2Mgh14 | |
| 2 | K238 | D2Mit7 | |
| 2 (5) | K280 | D5Mgh7 | |
| 2 | K374 | D2Wox8 | |
| 2 | K376 | D2Wox34 | |
| 2 | K384 | D2Wox1 | |
| | | | 11 |
| 3 | R142 | Svs2p | |
| 3 | K63 | D3Mit1 | |
| 3 | K66 | D3Mgh11 | |
| 3 | K68 | D3Mit10 | |
| 3 | K386 | D3Wox1 | |
| 3 | K393 | D3Wox16 | |
| 3 | K395 | D3Wox25 | |
| | | | 7 |
| 4 | R88 | Il-6 | |
| 4 | K147 | Eno | |
| 4 | K215 | Nos | |
| 4 | K253 | D4Mit9 | |
| 4 | K262 | D4Mit16 | |
| 4 | K264 | D4Mgh9 | |
| 4 | K267 | D4Mgh11 | |
| 4 | K547 | D4Rat9 | |
| | | | 8 |
| 5 | R44 | A2ug | |
| 5 | Cjun | Cjun | |
| 5 | K72 | D5Mgh15 | |
| 5 | K151 | D5Mgh5 | |
| 5 | K270 | D5Mgh1 | |
| 5 | K404 | D5Wox26 | |
| 5 | K412 | D5Wox17 | |
| | | | 7 |
| 6 | R99 | Ckb | |
| 6 | K73 | D6Mgh4 | |
| 6 | K74 | D6Mit5 | |
| 6 | K154 | D6Mit1 | |
| 6 | K155 | D6Mgh5 | |
| 6 | K156 | D6Mit4 | |
| 6 | K158 | D6Mit9 | |
| 6 | K159 | D6Pas1 | |
| 6 | K284 | D6Mgh2 | |
| 6 | K288 | D6Mit8 | |
| 6 | K289 | D6Mit2 | |
| 6 | K290 | D6Mit3 | |
| 6 | K416 | D6Wox2 | |
| 6 | K417 | D6Wox17 | |
| 6 | K420 | D6Wox10 | |
| 6 | K506 | D6Kyo4 | |
| 6 | K549 | D6Rat184 | |
| 6 | K554 | D6Rat160 | |
| 6 | K556 | D6Rat101 | |
| 6 | K557 | D6Rat31 | |
| 6 | K625 | D6Rat66 | |
| | | | 21 |
| 7 | K26 | D7Mit9 | |
| 7 | K77 | D7Mit7 | |
| 7 | K162 | D7Mgh7 | |
| 7 | K163 | D7Mgh9 | |
| 7 | K300 | D7Mit16 | |
| 7 | K427 | D7Wox28 | |
| 7 | K428 | D7Wox15 | |
| 7 | K435 | D7Wox2 | |
| | | | 8 |
| 8 | R102 | Apoc3 | |
| 8 | K84 | D8Mit4 | |
| 8 | K116 | D8Mit2 | |
| 8 | K117 | D8Mgh11 | |
| 8 | K166 | D8Mgh4 | |
| 8 | K437 | D8Wox23 | |
| 8 | K444 | D8Wox12 | |
| 8 | K560 | D8Rat51 | |
| | | | 8 |
| 9 | R27 | Cryg | |
| 9 | Inhb | Inhb | |
| 9 | K87 | D9Mit1 | |
| 9 | K88 | D9Mgh2 | |
| 9 | K171 | Aep2 | |
| 9 | K451 | D9Wox20 | |
| 9 | K453 | D9Mgh5 | |
| | | | 7 |
| 10 | R126 | Aep | |
| 10 | Il-4 | Il-4 | |
| 10 | K30 | D10Mgh2 | |
| 10 | K32 | D10Mit10 | |
| 10 | K119 | D10Mgh6 | |
| 10 | K644 | D10Rat4 | |
| 10 | K646 | D10Rat8 | |
| | | | 7 |
| 11 | R22 | Smst | |
| 11 | K39 | D11Mgh5 | |
| 11 | K91 | D11Mit2 | |
| 11 | K120 | D11Mgh3 | |
| 11 | K459 | D11Wox4 | |
| 11 | K516 | D11Rat22 | |
| | | | 6 |
| 12 | K42 | D12Mit5 | |
| 12 | K179 | D12Mit2 | |
| 12 | K182 | D12Mgh4 | |
| 12 | K183 | D12Mit4 | |
| 12 | K468 | D12Wox15 | |
| | | | 5 |
| 13 | Atpa | Atpa | |
| 13 | K11 | D13UW | |
| 13 | K93 | D13Mit1 | |
| 13 | K122 | D13Mgh8 | |
| 13 | K189 | D13Mgh7 | |
| | | | 5 |
| 14 | R40 | Alb | |
| 14 | R130 | Gck | |
| 14 | K99 | D14Mgh2 | |
| 14 | K100 | D14Mgh1 | |
| 14 | K128 | Csna | |
| 14 | K130 | D14Mgh3 | |
| 14 | K337 | D14Mgh4 | |
| 14 | K584 | D14Rat65 | |
| 14 | K588 | D14Rat37 | |
| | | | 9 |
| 15 | K45 | D15Mgh3 | |
| 15 | K102 | D15Mgh6 | |
| 15 | K123 | D15Mgh5 | |
| 15 | K482 | D15Wox5 | |
| 15 | K483 | D15Wox3 | |
| | | | 5 |
| 16 | K48 | D16Mgh4 | |
| 16 | K49 | D16Mit2 | |
| 16 | K170 | D9Mgh1 | |
| 16 | K486 | D16Wox7 | |
| | | | 4 |
| 17 | K35 | D17Mgh3 | |
| 17 | K36 | D17Mgh5 | |
| 17 | K104 | D17Mit7 | |
| 17 | K105 | D17Mit5 | |
| 17 | K491 | D17Wox8 | |
| | | | 5 |
| 18 | R66 | Olf | |
| 18 | R98 | Gjai | |
| 18 | Mit9 | D18Mit9 | |
| 18 | K12 | D18Mgh3 | |
| 18 | K202 | D18Mit4 | |
| 18 | K511 | D18Kyo1 | |
| 18 | K559 | D18Rat44 | |
| | | | 7 |
| 19 | K206 | D19Mit7 | |
| 19 | K569 | D19Rat12 | |
| 19 | K572 | D19Rat3 | |
| 19 | K575 | D19Rat34 | |
| | | | 4 |
| 20 | R145 | Tnf | |
| 20 | K108 | D20Mgh1 | |
| 20 | K127 | Prkacn | |
| | | | 3 |
| X | Mgh3 | DXMgh3 | |
| X | K15 | DXMgh1 | |
| X | R87 | Pfkfb | |
| X | R47 | Ar | |
| X | K657 | DXRat17 | |
| X | K654 | DXRat19 | |
| X | K661 | DXRat103 | |
| X | K661 | DXRat103 | |
| X | K364 | DXWox29 | |
| X | K352 | DXWox17 | |
| X | K359 | DXWox24 | |
| | | | 11 |
| Total | | | 160 |

### REFERENCES

1. She, J -X. and M. P. Marrron. Genetic susceptibility factors in typ1 diabetes: linkage, disequilibrium and functional analyses, Cur. Opin. Immunol., 10, 1998, 682-689
2. Morahan, G., Huang, D., Ymer, S. I. et al. Linkage disequilibrium of a type 1 diabetes susceptibility locus with a regulatory IL12B allele. Nature Genet. 27, 2001, 218-221
3. Klöting, I. and L. Vogt. BB/O(ttawa)K (arlsburg) rats: Features of a subline of diabetes-prone BB rats. Diabetes Res., 1991,18,79-87
4. Lühder, F., Woltanski, K. P., Hamann, J. et al. Detection of antibodies against both isoforms of glutamate decarboxylase in BB/OK rats by western blotting and immuno trapping enzyme activity assay. Diabetes Res. 20, 1992, 97-107
5. Ziegler, B., Witt, S., Kohnert, K. D. et al. Characterization of monoclonal islet cell reactive autoantibodies from the diabetic Biobreeding (BB/OK) rat. Acta Diabetol. 30, 1993, 201-206
6. Schröder, D., Schmidt, S., Klöting, I. and B. Hehmke. Effect of syngeneic islet antigen administration on complement-dependent antibody-mediated cytotoxicity to islet cells and diabetes onset in diabetes-prone BB/OK rats. Autoimmunity 14,1993, 283-289
7. Vogt, L. and I. Klöting. Genetic analysis of frequencies of phenotypes in the spontaneously diabetic BB rat—a main animal model of autoimmune type-I-diabetes. Diabetes Res., 22, 1993, 105-113
8. Klöting, I., Stark, O. and U. Fischer. Incidence of diabetes in F2 and first backcross hybrids of BB rats of different origin. In: Lessons from animal diabetes II/Eds. Shafrir, Renold, -London, Paris, J. Libbey, 1988, 85-87
9. Klöting, I., and Vogt, L. (1990). Coat colour phenotype, leucopenia and insulin-dependent diabetes mellitus in BB rats. Diabetes Res. 15, 37-39
10. Klöting, I., Stielow, M. and L. Vogt. Development of new animal models in diabetes research: spontaneously hypertensive-diabetic rats. Diabetes Res. 29, 1995, 127-138
11. Klöting, I. and P. Kovacs. Crosses between diabetic BB/OK and wild rats confirm that a 3^{rd} gene is essential for diabetes development. Acta Diabetol. 35, 1998, 109-111
12. Klöting, I., Voigt, B. and P. Kovács. Comparison of genetic variability at microsatellite loci in wild rats and inbred rat strains (Rattus norvegicus). Mamm. Genome. 8, 1997, 589-591
13. Klöting, I., Vogt, L. and T. Serikawa. Locus on chromosome 18 cosegregates with diabetes in the BB/OK rat subline. Diabet. Metab. 21, 1995, 338-344
11. Klöting, I., S. Schmidt and P. Kovacs. Mapping of novel diabetes predisposing and protecting genes in the spontaneously diabetic BB/OK rat. Biochem. Biophys. Res. Commun. 245, 1998, 483-486
15. Klöting, I. and P. Kovacs. Phenotypic differences between diabetes-prone BB rat sublines cosegregate with loci on chromosomes X and 10. Biochem. Mol. Biol. Int. 45, 1998, 865-870
16. Klöting, I. and P. Kovacs. Genes of the immune system cosegregate with the age at onset of diabetes in the BB/OK rat. Biochem. Biophys. Res. Commun. 242, 1998, 461-463
17. Klöting, I. and P. Kovacs. Loci of the immune system are implicated in diabetes frequency and age at onset of diabetes in BB rats. J. Exp. Anim. Sci. 41, 2000, 19-21
18. Klöting, I., Vogt, L., Reetz, I. C. et al. New congenic rat strains for the separate study of MHC and non-MHC genetic effects in the development of diabetes in BB rats. Diabetes Res. 15, 1990, 41-45
19. Voigt, B., Berg, S., Kovács, P. et al. Congenic spontaneously diabetic hypertensive BB.SHR rats. Transplant. Proc. 29, 1997, 1677-1678
20. Klöting, I., Kovacs, P. and B. Kuttler. Phenotypic consequences after restoration of lymphopenia in the diabetes-prone BB/OK rat. Biochem. Biophys. Res. Commun. 239, 1997, 106-110
21. Klöting, I. Voigt, B. and P. Kovacs. Metabolic features of newly established congenic diabetes-prone BB.SHR rat strains. Life Sci. 62, 1998, 973-979
22. Kovacs, P. and I. Klöting. Congenic strain confirms putative quantitative trait locus for body weight in the rat. Mamm. Genome 9, 1998, 294-296
23. van den Brandt, J., Kovacs, P. and I. Klöting. Congenic diabetes-prone BB.Sa and BB.Xs rats differ from their progenitor strain BB/OK in frequency and severity of insulin-dependent diabetes mellitus. Biochem. Biophys. Res. Commun. 263, 1999, 843-847
24. Klöting, I., van den Brandt, J. and B. Kuttler. Genes of SHR rats protect spontaneously diabetic BB/OK rats from diabetes: Lessons from congenic BB.SHR rat strains. Biochem. Biophys. Res. Commun. 283, 2001, 399-405
25. Podolin, P. L., Denny, P., Lord, C. J. et al. Congenic mapping of the insulin-dependent diabetes (idd) gene, Idd10, localises two genes mediating the Idd10 Effect and eliminates the candidate Fcgr1. J. Immunology 159, 1997, 1835-1843
26. Podolin, P. L., Denny, P., Armitage, N. et al. Localisation of two insulin-dependent diabetes genes to the Idd10 region on mouse chromosome 3. Mamm. Genome 9, 1998, 283-286
27. Lyons, P. A., Hancock, W. W., Denny, P. et al. The NOD Idd9 genetic interval influences the pathogenicity of insulitis and contains molecular variants of Cd30, Tnfr2, and Cd137. Immunity 13, 2000, 107-115
28. McDuffie, M. Derivation of diabetes-resistant congenic lines from the nonobese diabetic mouse. Clinical Immunology 96, 2000, 119-130
29. Norman, R. A., Dzielak, D. J., Bost, K. L. et al. Galloway. Immune dysfunction contributes to the aetiology of spontaneous hypertension. J. Hypertens. 3, 1985, 261-268
30. Fannon, L. D., Braylan, R. C. and M. I. Phillips. Alterations of lymphocyte populations during development in the spontaneously hypertensive rat. J. Hypertens. 10, 1992, 629-634
31. Ofosu-Appiah, W. and C. Ruggiero. Abnormal activation and loss of suppressor T cells in the spontaneously hypertensive rat. Cell Immunol. 145, 1992, 130-145
32. Klöting, I., van den Brandt, J. and B. Kuttler Different and gradual protection from spontaneous diabetes by alleles of SHR and Wild rats in congenic BB.SHR and BB.KWR rat strains. 10^{th} International Symposium on SHR and Molecular Medicine, 2.-4.5.2001, Berlin-Buch. J. Mol. Med. 79, 2001, B12, Abstr.
33. Watanabe, T. K., Bihoreau, M. T., McCarthy, L. C. et al. A radiation hybrid map of the rat genome containing 5,255 markers. Nat. Genet. 22, 1999, 27-36
34. Tomas, E., Lin, Y. S., Dagher, Z. et al. Hyperglycemia and insulin resistance: possible mechanisms. Ann. N.Y. Acad. Sci. 967, 2002, 43-51
35. Lawlor, M. A. and D. R. Alessi. PKB/Akt: a key mediator of cell proliferation, survival and insulin response. J. Cell Sci. 114, 2001, 2903-2910
36. Summers, S. A., Yin, V. P., Whiteman, E. L. et al. Signaling pathways mediating insulin-stimulated glucose transport. Ann. N.Y. Acad. Sci. 892, 1999, 169-186
37. Lucke, S., Klöting, I., Pusch, A., et. al. Endocrine pancreas histology of congenic BB-rat strains with reduced incidence after genetic manipulation on chromosoms 4,6 and X. Autoimmunity 36, 2003, 143-149)
38. Follak, N., Klöting, I. Ganzer, D. et al. Scanning electron microscopic examinations on retarted bone defect healing in spontaneously diabetic BB/O(ttawa)K(arlsburg) rats. Histol. Histopathol. 18, 2003,111-120
39. Pondel, M. Calcitonin and calcitonin receptors: bone and beyond. Int. J. Exp. Pathol. 81, 2001, 405-422
40. Ahren, B. Autonomic regulation of islet hormone secretion-Implications for health and disease. Diabetologia 43, 2000, 393-410
41. Rabinovitch, A., Suarez-Pinzon, W. L., Sooy, K. et al. Expression of calbindin-D(28k) in a pancreatic islet beta-cell line protects against cytokine-induced apoptosis and necrosis. Endocrinology 142, 2001, 3649-3655
42. Sooy, K., Schermerhorn, T., Noda, M. et al. Calbindin-D(28k) controls (Ca(2+) (i) and insulin release. Evidence obtained from calbindin-d(28k) knockout mice and beta cell lines. J. Biol. Chem. 274, 1999, 34343-34349
43. Lee, J. S., See, R. H., Galvin, K. M. et al. Functional interactions between YY1 and adenovirus E1A. Nucleic Acids Res. 23, 1995, 925-931
44. Bushmeyer, S. M. and M. L. Atchison. Identification of YY1 sequences necessary for association with the nuclear matrix and for transcriptional repression functions. J. Cell Biochem. 68, 1998, 484-499
45. Galvin, K. M. and Y. Shi. Multiple mechanisms of transcriptional repression by YY1. Mol. Cell Biol. 17,1997, 3723-3732
46. Houbaviy, H. B., Usheva, A., Shenk, T. et al. Cocrystal structure of YY1 bound to the adeno-associated virus P5 initiator. Proc. Natl. Acad. Sci. 93, 1996, 13577-13582
47. Lewis, B. A., Tullis, G., Seto, E. et al. Adenovirus E1A proteins interact with the cellular YY1 transcription factor. J. Virol. 69, 1995,1628-1636
48. Yang, W. M., Inouye, C., Zeng, Y. et al. Transcriptional repression by YY1 is mediated by interaction with a mammalian homolog of the yeast global regulator RPD3. Proc. Natl. Acad. Sci. USA 93,1996,12845-12850
49. Thomas, J. M. and E. Seto. Unlocking the mechanisms of transcription factor YY1: are chromatin modifying enzymes the key. Gene 236, 1999, 197-208
50. Shrivastava, A., Saleque, S., Kalpana, S. et al. Inhibition of transcriptional regulator Yin-Yang-1 by association with c-Myc. Science 262,1993,1889-1892
51. Austen, M., Luscher, B. and J. M. Luscher-Firzlaff. Characterization of the transcriptional regulator YY1. The bipartite transactivation domain is dependent of interaction with the TATA box-binding protein, transcription factor IIB, TAFII55, or c-AMP-responsive element-binding protein (CBP)-binding protein. J. Biol. Chem. 272, 1997,1709-1717
52. Seto, E., Lewis, B. and T. Shenk. Interaction between transcription factor SP1 and YY1. Nature 365, 1993, 462-464
53. Lee, J. S., Galvin, K. M. and Y. Shi. Evidence of physical interaction between the zinc-finger transcription factors YY1 and SP1. Proc. Natl. Acad. Sci. USA 90,1993, 6145-6149
54. Lee, J. S., Zang, X. and Y. Shi. Differential interaction of the CREB/ATF family of transcription factors with p300 and adenovirus E1A. J. Biol. Chem. 271, 1996, 17666-17674
55. Lee, J. S., Galvin, K. M., See, R. H. et al. Relief of YY1 transcriptional repression by adenovirus E1A is mediated by E1A-associated protein p300. Genes Devel. 9, 1995, 1188-1198
56. Hyde-DeRuyscher, R. P., Jennings, E. and T. Shenk. DNA binding sites for the transcriptional activator/repressor YY1. Nucl. Acid Res. 23, 1995, 4457-4465
57. Tauton, J., Hassig, C. A. and S. L. Schreiber. A mammalian histone deacetylase related to the yeast transcriptional regulator Rpd3p. Science 272,1996, 408-411
58. Yang, W. M., Yao, Y. L., Sun, J. M. et al. Isolation and characterization of c-DNAs corresponding to an additional member of the human histone deacetylase gene family. J. Biol. Chem. 272, 1997, 28001-28007
59. Hassig, C. A. and S. L. Schreiber. Nuclear histone acetylases and deacetylases and transcriptional regulation: HATs off to HDACs. Curr. Opin. Chem. Biol. 1, 1997, 300-308
60. Hassig, C. A., Tong J. K., Fleischer T. C. et al. A role of histone deacetylase activity in HDAC1-mediated transcriptional repression. Proc. Natl. Acad. Sci. USA 95, 1998, 3519-3524
61. Shi, Y., Seto, E., Chang, L. et al. Transcriptional repression by YY1, a human GLI-Krueppel-related protein and relief of repression by adenovirus E1A protein. Cell 67, 1991, 377-388
62. Usheva, A. and T. Shenk. TATA-binding-protein-independent initiation: YY1, TFIIB and RNA polymerase II direct basal transcription on supercoiled template DNA. Cell 76,1994, 1115-1121
63. Chiang, C. M. and R. G. Roeder. Cloning of an intrinsic human TFIID subunit that interacts with multiple transcriptional activators. Science 267,1995, 531-536
64. Seto, E., Shi, Y. and T. Shenk. YY1 is an initiator sequence-binding protein that directs and activates transcription in vitro. Nature 354,1991, 241-245
65. Kwok, R. P., Lundblad, J. R., Chrivia, J. C. et al. Nuclear protein CBP is a coactivator for the transcription factor CREB. Nature 370,1994,223-226
66. Yao, Y. L., Yang, W. M. and E. Seto. Regulation of the transcription factor YY1 by acetylation and deacetylation. Mol. Cell Biol. 17, 2001, 5979-5991
67. van den Brandt, J., Kovacs, P. and I. Klöting. Features of the metabolic syndrome in the spontaneously hypertriglyceridemic W(istar) O(ttawa)K(arlsburg)W(RT1^{u}) rat. Metabolism 49, 2000, 1140-44
68. van den Brandt, J., Kovacs, P. and I. Klöting. Metabolic features in disease-resistant as well as in hypertensive SHR and newly established obese WOKW inbred rats. Int. J. Obesity 24, 2000, 1618-1622
69. Rosmalen, J. G., Leenen, P. J., Pelegri, C. et al. Islet abnormalities in the pathogenesis of autoimmune diabetes. Trends. Endocrinol. Metab. 13, 2002, 209-214
69b. Nishiyama C., Yokota T., Nishiyama M. et al. Molecular clonong of the rat transcription factor YY1. Biosci. Biotechnol. Biochem. 67, 2003, 654-658
70. Kolb, H. Benign versus destructive insulitis. Diabetes Metab. Rev. 13, 1997, 139-146
71. Foulis, A. K., Farquharson, M. A. and A. Meager. Immunoreactive alpha-interferon in insulin-secreting beta cells in type 1 diabetes mellitus. Lancet 2(8573), 1987,1423-1427
72. Huang, X., Yuang, J., Goddard, A. et al. Interferon expression in the pancreas of patients with type 1 diabetes. Diabetes 44, 1995, 658-664
73. Foulis, A. K., McGill, M. and M. A. Farquharson. Insulitis in type 1 (insulin-dependent) diabetes mellitus in man: macrophages, lymphocytes, and interferon-γ containing cells. J. Pathol. 165, 1991, 97-103
74. Yamagata, K., Nakajima, H., Tomita, K. et al. Dominant TCR α chain clontypes and interferon-γ are expressed in the pancreas of patients with recent-onset insulin-dependent diabetes mellitus. Diabetes Res. Clin. Pract. 34, 1996,37-46
75. Huang, X., Hultgren, B., Dybdal, N. et al. Islet expression of interferon-α precedes diabetes in both the BB rat and streptozotocin-treated mice. Immunity 1, 1994,469-478
76. Campbell, I. L., Hobbs, M. V., Dockter, J. et al. Islet inflammation and hyperplasia induced by the pancreatic islet-specific overexpression of interleukin-6 in transgenic mice. Am. J. Pathol. 145, 1994, 157-166
77. DiCosmo, B. F., Picarella, D. and R. A. Flavell. Local production of human IL-6 promotes insulitis but retards the onset of insulin-dependent diabetes mellitus in non-obese diabetic mice. Int. Immunol. 6, 1994,1829-1837
78. Wogensen, L., Lee, M. and N. Sarvetnick. Production of interleukin 10 by islet cells accelerates immune-mediated destruction of β cells in non-obese diabetic mice. J. Exp. Med. 179, 1994,1379-1384
79. Moritani, M., Yoshimoto, K., Tashiro, F. et al. Transgenic expression of IL-10 in pancreatic islet A cells accelerates autoimmune diabetes in non-obese diabetic mice. Int. Immunol. 6, 1994,1927-1936
80. Mueller, R., Krahl, T. and N. Sarvetnick. Pancreatic expression of interleucin-4 abrogates insulitis and autoimmune diabetes in non-obese diabetic (NOD) mice. J. Exp. Med. 184, 1996, 1093-1099
81. Rabinovitch, A. and W. L. Suarez-Pinzon. Cytokines and their roles in pancreatic islet β-cell destruction and insulin-dependent diabetes mellitus. Biochem. Pharmacol. 55, 1998, 1139-1149
82. Klöting, I., van den Brandt, J. and B. Kuttler. Genes of SHR rats protect spontaneously diabetic BB/OK rats from diabetes: Lessons from congenic BB.SHR rat strains. Biochem. Biophys. Res. Commun. 283, 2001, 399-405
83. Guo, J., Casolaro, V., Seto, E., et al. Yin-Yang 1 activates interleukin-4 gene expression in T cells. J. Biol. Chem. 276, 2001, 48871-48878
84. Ye, J., Cippitelli, M., Dorman, L. et al. The nuclear factor YY1 suppresses the human gamma interferon promoter through two mechanisms: inhibition of AP1 binding and activation of a silencer element. Mol. Cell Biol. 16, 1996, 4744-4753
85. Hänninen, A., Jalkanen, S., Salmi, M. et al. Macrophages, T-cell receptor usage, and endothelial cell activation in the pancreas at the onset of insulin-dependent diabetes mellitus. J. Clin. Invest. 90, 1992,1901-1910
86. Kay, T. W. H., Campbell, I. L., Oxbrow, L. et al. Overexpression of class I major histocompatibility complex accompanies Insulitis in the non-obese diabetic mouse and is prevented by anti-interferony-antibody. Diabetologia, 34, 1991, 779-785
87. Garban, H. J. and B. Bonavida. Nitric oxide inhibits the transcription repressor Yin-Yang 1 binding activity at the silencer region of the Fas promoter: a pivotal role for nitric oxide in the up-regulation of Fas gene expression in human tumor cells. J. Immunol. 167, 2001, 75-81
88. Like, A. A., Butler, L., Williams, R. M. et al. Spontaneous autoimmune diabetes mellitus in the BB rat. Diabetes 31 (Suppl. 1), 1982, 7-13
89. Janeway, C. A. and P. Travers. Immunologie. Spektrum Akademischer Verlag Heidelberg, Berlin, Oxford. 1997; 2 Auflage: 208-236.
90. Reizis, B. and P. Leder. Expression of the mouse Pre-T cell receptor a Gene is controlled by an upstream region containing a transcriptional enhancer. J. Exp. Med. 189,1999,1669-1678
91. Iritani, B. M. and R. N. Eisenman. c-Myc enhances protein synthesis and cell size during B lymphocyte development. Proc. Natl. Acad. Sci. USA, 96, 1999, 13180-13185
102. Dang, C., Resar, L., Emison, E. et al. Function of the c-Myc oncogenic transcription factor. Exp. Cell Res., 253, 1999, 63-77
103. Austen, M., Cerni, C., Luscher-Firzlaff, J. M. and B. Luscher. YY1 can inhibit c-Myc function through a mechanism requiring DNA binding of YY1 but neither its transactivation domain nor direct interaction with c-Myc. Oncogene 30, 1998, 511-520
104. Menssen, A. and H. Hermeking. Characterization of the c-Myc-regulated transcriptome by SAGE: Identification and analysis of c-Myc target genes. Proc. Natl. Acad. Sci. U S A 99, 2002, 6274-6279
105. Schwab, M., Varmus, H. E., Bishop, J. M. et al. Chromosome localisation in normal human cells and neuroplastomas of a gene related to c-Myc. Nature 308,1984, 288-291
106. Nau, M. M., Brooks, B. J., Battey, J. et al. L-myc, a new myc-related gene amplified and expressed in human small cell lung cancer. Nature 318, 1985, 69-73
107. Sugiyama, A., Kume, A., Nemoto, K. et al. Isolation and characterization of s-myc, a member of the rat myc gene family. Proc. Natl. Acad. Sci. U S A 86, 1989, 9144-9148
108. Asker, C., Steinitz, M., Andersson, K. et al. Nucleotide sequence of the rat B-myc gene. Oncogene 4, 1989,1523-1527
109. Blackwell, T. K., Huang, J., Ma, A. et al. Binding of myc proteins to canonical and noncanonical DNA sequences. Mol. Cell Biol. 13,1993, 5216-5224
110. Blackwood, E. M. and R. N. Eisenmann. Max: a helix-loop-helix zipper protein that forms a sequence-specific DNA-binding complex with Myc. Science 251,1991, 1211-1217
111. Grandori, C., Cowley, S. M., James, L. P. et al. The Myc/Max/Mad network and the transcriptional control of cell behaviour. Annu. Rev. Cell Dev. Biol. 16, 2000, 653-699
112. Klöting, I. and L. Vogt. Influence of age and metabolic state of mothers on the life expectancy of non-diabetic offspring in diabetes-prone BB/OK rats. Proceedings of the Fifth FELASA Symposium, Ed. Bunyan, J., 1994, 265-269
131. Stocco, D. M. Tracking the role of a StAR in the sky of the new milennium. Mol. Endocrinol. 15, 2001, 1245-1254
132. Nackley, A. C., Shea-Eaton, W., Lopez, D. et. al. Repression of the steroidogenic acute regulatory gene by the multifunctional transcription factor Yin Yang 1. Endocrinology 143, 2002,1085-1096
133. Christenson, L. K., Osborne, T. F., McAllister, J. M. et al. Conditional response of the human steroidogenic acute regulatory protein gene promoter to sterol regulatory element binding protein-1a. Endocrinology 142, 2001, 28-36
134. Ericsson, J., Usheva, A. and P. A. Edwards. YY1 is a negative regulator of transcription of three sterol regulatory element-binding protein-responsive genes. J. Biol. Chem. 274, 1999, 14508-14513
135. Amemiya-Kudo, M., Shimano, H., Yoshikawa, T. et al. Promoter analysis of the mouse sterol regulatory element-binding protein-1c gene. J. Biol. Chem. 275, 2000, 31078-31085
136. Amemiya-Kudo, M., Shimano, H., Hasty, A. H. et al. Transcriptional activities of nuclear SREBP-1a, -1c. and -2 to different target promoters of lipogenic and cholesterogenic genes. J. Lipid Res. 43, 2002, 1220-1235
137. Shea-Eaton, W., Lopez, D. and M. P. McLean. Yin Yang 1 protein negatively regulates high-density lipoprotein receptor gene transcription by disrupting binding of sterol regulatory element binding protein to the sterol regulatory element. Endocrinology 142, 2001, 49-58
138. Bennett, M. K. and T. F. Osborne. Nutrient regulation of gene expression by the sterol regulatory element binding proteins: increased recruitment of gene-specific coregulatory factors and selective hyperacetylation of histone H 3 in vivo. Proc. Natl. Acad. Sci. USA 97, 2000, 6340-6344
139. Hasty, A. H., Shimano, H., Yahagi, N. et al. Sterol regulatory element-binding protein-1 is regulated by glucose at the transcriptional level. J. Biol. Chem. 275, 2000, 31069-31077
140. Sun, L., Halaihel, N. et al. Role of sterol regulatory element-binding protein 1 in regulation lipid metabolism and glomerulosclerosis in diabetes mellitus. J. Biol. Chem. 277, 2002, 18019-18927
141. Sewter, C., Berger, D., et al. Human obesity and type 2 diabetes are associated with alterations in SREBP 1 isoform expression that are reproduced ex vivo by tumor necrosis factor. Diabetes 51, 2002,1035-1041
142. Hua, X., Yokoyama, C., Wu, J. et al. SREBP-2, a second basic-helix-loop-helix-leucine zipper protein that stimulates transcription by binding to a sterol regulatory element. Proc. Natl. Acad. Sci. USA 90, 1993,11603-11607
143. Lund, J. and H. F. DeLuca. Biologically active metabolite of vitamin D3 from bone, liver, and blood serum. J Lipid Res 7, 1966, 739-744
144. Löffler, G. and P. E. Petrides. Biochemie und Pathobiochemie. Springer Verlag Berlin, Heidelberg, N.Y. 1998; 6. Auflage: 657-659
145. Barker, A. R., McDonnell, D. P., Huges, M. et al. Cloning and expression of full length cDNA and coding human vitamin D receptor. Proc. Nat. Acad. Sci. 85, 1988, 3294-3298
146. Brumbaugh, P. F. and Haussler M. R. Nuclear and cytoplasmatic binding components for vitamin D metabolites. Life Sci 1975; 16: 353-362.
147. DeLuca H. F. and m. T. Cantorna. Vitamin D: its role and uses in immunology. FASEB J 2001; 15: 2579-2585.
148. Guo B., Aslam F., van Wijnen A. J. et al. YY1 regulates vitamin D receptor/retinoid x receptor mediated transactivation of the vitamin D responsive osteocalcin gene. Proc Natl Acad Sci USA 1997; 94: 121-126.
149. Raval-Pandya M., Dhawan P., Barletta F. et al. YY1 represses Vitamin D receptor mediated 25-Hydroxyvitamin D3 24-Hydroxylase transcription: relief of repression by CREB-binding protein. Mol Endocrinology 2001; 15: 1035-1046.
150. Kasuga H., Hosogane N., Matsuoka K. et al. Characterization of transgenic rats constitutively expressing vitamin D-24-hydroxylase gene. Biochem Biophys Res Commun 2002; 297(5): 1332-1338.
151. Chang T. J., Lei H. H., Yeh J. I. et al. Vitamin D receptor gene polymorphisms influence susceptibility to type 1 diabetes mellitus in the Taiwanese population. Clin Endocrinology 2000; 52: 575-580.
152. Li Y. C., Kong J., Wei M. et al. 1,25 Dihydroxyvitamin D3 is a negative endocrine regulator of the renin-angiotensin-system. J Clin Invest 2002; 110: 229-238.
153. Bhalla S. S., Robitaille L. and M. Nemer. Cooperative action by GATA-4 and YY1 of the cardiac b-type natriuretic peptide promotor. 2001 J Biol Chem 276: 11439-11445.
154. Charron F. and M. Nemer. GATA transcription factors and cardiac development. Semin Cell Dev Biol 1999; 10(1): 85-91.
155. Vincent C. K., Gualberto A., Patel C. V. et al. Different regulatory sequences control creatine kinase-M gene expression in directly injected skeletal and cardiac muscle. Mol Biol Cell 1993; 13(2): 1264-1272.
156. Lee T. C., Zhang Y. and R. J. Schwartz. Bifunctional transcriptional properties of YY1 in regulating muscle actin and c-myc gene expression during myogenesis. Oncogene 1994; 9(4): 1047-1052.
157. Chen C. Y. and R. J. Schwartz. Competition between negative acting YY1 versus positive acting serum response factor and tinman homologue Nkx-2.5 regulates cardiac alpha-actin promotor activity. Mol Endocrinol 1997; 11(6): 812-822.
158. MacLellan W. R., Lee T. C., Schwartz R. J. et al. Transforming growth factor-beta response elements of the alpha-actin gene. Combinatorial action of serum response factor, YY1, and SV40 enhancer-binding protein, TEF-1. J Biol Chem 1994; 269(24): 16754-16760.
159. Patten M., Hartogenis W. F. and C. S. Long. Interleukin 1β Is a negative transcriptional regulator of alpha1-adrenic induces gene expression in cultured cardiac myocytes. J Biol Chem 1996; 271(35): 21134-21141.
160. Tan, T. P., Nonaka, K., Nuckolls, G. H., Liu, Y. H. Maxson, R. E., Slavkin, H. C. and L. Shum. YY1 activates Msx2 gene independent of bone morphogenetic protein signalling. Nucl. Acids Res. 30, 2002, 1213-1223
161. Lumsden, A. and R. Krumlauf. Patterning the vertebrate neuraxis. Science 274, 1996, 1109-1115
162. Chariot, A., Gielen, J., Merville, M. P. and V. Bours. The homeodomain-containing proteins: an update on their interacting partners. Biochem. Pharmacol. 58, 1999, 1851-1857
163. Bendall, A. J. and C. Abate-Shen. Roles for Msx and Dlx homeoproteins in vertebrate development. Gene 247, 2000,17-31.
164. Liu, Y. H., Ma, L., Kundu, R., Ignelzi, M., Sangiorgi, F., Wu, L., Luo, W., Snead, M. L. and R. Maxson. Function of the Msx2 gene in the morphogenesis of the skull. Ann. N.Y. Acad. Sci. 785, 1996, 48-58
165. Cohen, M. M., Jr. Craniofacial disorders caused by mutations in homeobox genes MSX1 and MSX2. J. Craniofac. Genet Dev. Biol. 20, 2000, 19-25
166. Pan, Z., Lichtler, A. C. and W. B. Upholt. DNase I hypersensitive sites in the chromatin of the chicken Msx2 gene differ in anterior and posterior limb mesenchyme, calvarial osteoblasts and embryonic fibroblasts. Biochem. Mol. Biol. Int. 46, 1998, 549-557
167. Donohoe, M. E., Zhang, X., McGinnis, L., Biggers, J., Li, E. and Y. Shi. Targeted disruption of mouse Yin Yang 1 transcription factor results in peri-implantation lethality. Mol. Cell. Biol. 19, 1999, 7237-7244
168. Jansen, B. and U. Zangemeister-Wittke. Antisense therapy for cancer-the time of truth. Lancet Oncol. 3, 2002, 672-683
169. Chen, Y. A novel single-stranded DNA expression vector. Expert Opin. Biol. Ther. 2, 2002, 735-740

1. A protein, characterised in that it displays the amino acid sequence represented in SEQ ID NO:4.

2. A protein that is a homologue of the protein according to claim 1 and which displays an amino acid sequence homologous to the sequence represented in SEQ ID NO:4, wherein the protein displays arginine at position 303 and lysine at position 311.

3. The protein according to claim 2, which displays a degree of homology of at least 95%.

4. The protein according to claim 3, which displays a degree of homology of at least 97%.

5. The protein according to claim 3 or 4, which displays a degree of homology of at least 99%.

6. A peptide, characterised in that it is a fragment of the protein according to claims 1 to 5 and displays an amino acid sequence that contains the sequence region that includes the amino acid positions 303 and 311 in SEQ ID NO:4.

7. The peptide according to claim 6, characterised in that it displays a length of 53 to 315 amino acids.

8. A nucleic acid, characterised in that it encodes a protein or a peptide according to claims 1 to 7.

9. The nucleic acid according to claim 8, characterised in that it displays the nucleic acid sequence represented in SEQ ID NO:3.

10. The nucleic acid according to claim 8, characterised in that it displays a nucleic acid sequence that contains the sequence region that includes the nucleic acid positions 979-981 and 1003-1005 in SEQ ID NO:3.

11. An antibody that is directed against a protein or peptide according to claims 1 to 7.

12. The antibody according to claim 11, characterised in that it is a monoclonal or polyclonal antibody.

13. A method to determine the tendency of falling ill with type 1 diabetes, wherein genomic DNA is amplified from isolated mononuclear blood cells, and sequenced, and where modifications of or deviations from the nucleic acid sequence are determined, this sequence coding for a protein with the amino acid sequence represented in SEQ ID NO:6, where deviations of codons (non-silent mutations) display increased tendency for falling ill with an autoimmune disease, type 2 diabetes, cancer or disruptions to the mineral and lipoid metabolism.

14. The method according to claim 13, wherein for amplification the following primers are used: K815-F/K817-R; K815-F/K875; K821-F/K817-R; K821-F/K870-R; K874-F/K870-R; K823-F/K825-R; K884-F/K806-R; K801-F/K804-R; K814-F/KK832-R; K828-F/K833-R; K831-F/K817-R; K815-F/K870-R; K815-F/K818-R; K816-F/K819-R; K834-F/K836-R, F15/R12; F15/R14; F15/R13; F57/R16; F57/R20; F57/R21; F59/RR25; F59/RR30; F59/R33; F95/RR34; F96/R39; F95/R48; F95/R50; F60/R7; F60/R8; F60/R66; F60/R67; F96/R76; F96/R77; F96/R81 F96/R83; F33/R1; F33/R4; F33/R15; F39/R28; F40/R3;
or F41/R5.

15. A method to determine the tendency of falling ill with type 1 diabetes, wherein RNA is isolated from isolated mononuclear blood cells or tissue biopsies, amplified and quantified, where enhanced/reduced expression indicates an increased/reduced tendency to fall ill with type 1 diabetes, autoimmune diseases in general, type 2 diabetes, cancer or disruptions to the mineral and lipoid metabolism.

16. Use of a protein or peptide according to claims 1 to 7 for the manufacture of a pharmaceutical compound.

17. Use of a nucleic acid according to claims 8 to 10 or of an antisense oligonucleotide of the same for the manufacture of a pharmaceutical compound.

18. A pharmaceutical composition that contains a nucleic acid according to claims 8 to 10, or an antisense oligonucleotide of the same.

19. A pharmaceutical composition that contains a protein and/or a peptide according to claims 1 to 7.

20. The composition according to claim 18 or 19, characterised in that it also contains pharmaceutically compatible excipients and/or carriers.

21. The composition according to claims 18 to 20, characterised in that the compound is formulated for intravenous application.

22. A transgenic non-human mammal, wherein the germ and somatic cells contain a nucleic acid or a nucleic acid segment which encodes a protein with the amino acid sequence shown in SEQ ID NO:2 or an amino acid sequence homologous to that, with a degree of homology of at least 95%, preferably at least 97%, and most preferably 99%, and where the homologous amino acid sequence displays methionine at position 303 and arginine at position 311.

23. The transgenic mammal according to claim 22, characterised in that it expresses the protein in the pancreas.

24. The mammal according to claim 22 or 23, characterised in that it is a rat.

25. Use of a transgenic or congenic non-human mammal, whose gamete and somatic cells contain a nucleic acid or a nucleic acid segment which encodes a protein with the amino acid sequence shown in SEQ ID NO:2 or an amino acid sequence homologous to that, with a degree of homology of at least 95%, preferably at least 97%, and most preferably 99%, and where the homologous amino acid sequence displays methionine at position 303 and arginine at position 311, for the purpose of identifying diabetes protective substances.

26. The use according to claim 25, wherein the degree of homology is at least 97%.

27. The use according to claim 25 or 26, wherein the degree of homology is at least 99%.

28. A kit for performing a method according to claims 13 to 15.


SEQUENCE LISTING (232 sequences)

SEQ ID NO: 1 - DNA, 2256 bp, Rattus norv.
  Promoter (1)..(72)
  CDS (73)..(1125): YY1 (BB/OK)
  misc_feature (955)..(1125): Zinc finger
  Intron (1126)..(1758)
  misc_feature (1759)..(1917): Zinc finger
  CDS (1759)..(1938): YY1 (BB/OK)
ccgcctcctcgcccgccctcccgcagcccaggagccgaggctgccgcggccgtggcggcggagccctcagccatggcctcgggcgacaccctctacattgccacggacggctcggagatgccagccgagatcgtggaactgcatgagattgaggtggagaccatcccggtggagactatcgagaccacggtggtgggcgaggaggaggacgacgacgaagacgacgaggatggtggcggcggagaccacggtggcgggggcggccacgggcacgctggccaccaccatcaccaccaccaccaccaccacccgcccatgatcgcgctgcagccgctggtcaccgacgacccgacccaagtgcaccaccaccaagaggtgattctggtgcagacgcgcgaggaggtagtgggtggcgacgactcggacgggctgcgcgccgaggacgggttcgaggaccagatcctcattccggtacccgcgccggccggcggagacgacgactacatcgagcagacgctggtcaccgtggcggcggccggcaagagcggtggcgggtcttcgtcgggcggcggccgcgttaagaagggcggcggcaagaagagcggcaagaagagttacctgggcagcggggccggcgcggcgggcggtggcggcgccgacccgggtaataagaagtgggaacagaagcaggtgcagatcaagaccctggagggcgagttctcggtcaccatgtggtcttcagatgaaaaaaaagatattgaccatgaaacagtggttgaagagcagatcattggggagaactcacctcctgattattctgaatatatgacaggcaagaaactccctcctggagggatacctggcattgacctctcagaccccaagcaactggcagaatttgccagaatgaagccaagaaaaattaaagaagatgatgctccaagaacaatagcttgccctcataaaggctgcacaaagatgttcagggataactctgctatgagaaagcatctgcacacccacggtcccagagtccacgtctgtgcagaatgtggcaaagcgttcgttgagagctcaaagctaaaacgacaccagctggttcatactggagaaaagccctttcaggtagagccagttcctgttccccaaactgcaagctagggtgctggtcagggtggttgatatcaagcactatggggcaccggttggggtattttattcccatccctcctgtctgcttgggttcctggttactgctcgggactgcaggtgttacagatgggggtggagggattatgcgaagcacccccacactaaatttctagcaggtttacaaaaactcaacagttttgttttgtagtgagtagtgtgttgaattactgatagagtgcttataagtgctgttggctacagctccaggtgacacttggtgctgcttatagaagactcgtgagttgacagttggcatcactaaatatcttaatcatctgtagtctacttcctagagtgtctctgaaaacactcaagctgtaaatttgcactcagcacagcccttctgtttctcaagaactagccatgggttgttagtatcagagatcccagtgtgtcagttctaaaataccctcagaagggttccagacgaggaaggaggcatgctcagcagaatagtaggtggtttccatctaagcagtgagccatcgatccccaggttctggtctcatttgccaagagggttgatatctggtttttccttgacagtgcacattcgaaggctgcgggaagcgcttttcactggacttcaatttgcgcacgcatgtgcgaatccataccggagacaggccctatgtgtgccccttcgacggttgtaataagaagtttgctcagtcaactaacctgaaatctcacatcttaacacacgctaaagccaaaaacaaccagtgaaaagaagagagaagaccttctcgaccccgggaagcctcttcaggagtgtgattgggaataaatatgcctctcctttgtatattatttctaggaagaattttaaaaatgaatcctacacacttaagggacatgttttgataaagtagtaaaaatttaaaaaaatactttaataagatgacattgctaagatgctctatcttgctctgtaatctcgtttcaaaaacaaggtgtttttgtaaagtgtggccccaacaggaggacaattcatgaacttcgcatcaaaagacaattctttatacaacagtgctaaaaatg

SEQ ID NO: 2 - protein, 411 aa, Rattus norv.
  misc_feature (955)..(1125): Zinc finger
  misc_feature (1759)..(1917): Zinc finger
MASGDTLYIATDGSEMPAEIVELHEIEVETIPVETIETTVVGEEEDDDEDDEDGGGGDHGGGGGHGHAGHHHHHHHHHHPPMIALQPLVTDDPTQVHHHQEVILVQTREEVVGGDDSDGLRAEDGFEDQILIPVPAPAGGDDDYIEQTLVTVAAAGKSGGGSSSGGGRVKKGGGKKSGKKSYLGSGAGAAGGGGADPGNKKWEQKQVQIKTLEGEFSVTMWSSDEKKDIDHETVVEEQIIGENSPPDYSEYMTGKKLPPGGIPGIDLSDPKQLAEFARMKPRKIKEDDAPRTIACPHKGCTKMFRDNSAMRKHLHTHGPRVHVCAECGKAFVESSKLKRHQLVHTGEKPFQCTFEGCGKRFSLDFNLRTHVRIHTGDRPYVCPFDGCNKKFAQSTNLKSHILTHAKAKNNQ

SEQ ID NO: 3 - DNA, 2256 bp, Rattus norv.
  Promoter (1)..(72)
  CDS (73)..(1125): YY1 (SHR)
  misc_feature (955)..(1125): Zinc finger
  Intron (1126)..(1758)
  misc_feature (1759)..(1917): Zinc finger
  CDS (1759)..(1938): YY1 (SHR)
ccgcctcctcgcccgccctcccgcagcccaggagccgaggctgccgcggccgtggcggcggagccctcagccatggcctcgggcgacaccctctacattgccacggacggctcggagatgccagccgagatcgtggaactgcatgagattgaggtggagaccatcccggtggagactatcgagaccacggtggtgggcgaggaggaggacgacgacgaagacgacgaggatggtggcggcggagaccacggtggcgggggcggccacgggcacgctggccaccaccatcaccaccaccaccaccaccacccgcccatgatcgcgctgcagccgctggtcaccgacgacccgacccaagtgcaccaccaccaagaggtgattctggtgcagacgcgcgaggaggtagtgggtggcgacgactcggacgggctgcgcgccgaggacgggttcgaggaccagatcctcattccggtacccgcgccggccggcggagacgacgactacatcgagcagacgctggtcaccgtggcggcggccggcaagagcggtggcgggtcttcgtcgggcggcggccgcgttaagaagggcggcggcaagaagagtggcaagaagagttacctgggcagcggggccggcgcggcgggcggtggcggcgccgacccgggtaataagaagtgggaacagaagcaggtgcagatcaagaccctggagggcgagttctcggtcaccatgtggtcttcagatgaaaaaaaagatattgaccatgaaacagtggttgaagagcagatcattggggagaactcacctcctgattattctgaatatatgacaggcaagaaactccctcctggagggatacctggcattgacctctcagaccccaagcaactggcagaatttgccagaatgaagccaagaaaaattaaagaagatgatgctccaagaacaatagcttgccctcataaaggctgcacaaagaggttcagggataactctgctatgaaaaagcatctgcacacccacggtcccagagtccacgtctgtgcagaatgtggcaaagcgttcgttgagagctcaaagctaaaacgacaccagctggttcatactggagaaaagccctttcaggtagagccagttcctgttccccaaactgcaagctagggtgctggtcagggtggttgatatcaagcactatggggcaccggttggggtattttattcccatccctcctgtctgcttgggttcctggttactgctcgggactgcaggtgttacagatgggggtggagggattatgcgaagcacccccacactaaatttctagcaggtttacaaaaactcaacagttttgttttgtagtgagtagtgtgttgaattactgatagagtgcttataagtgctgttggctacagctccaggtgacacttggtgctgcttatagaagacacgtgagttgacagttggcatcactaaatatcttaatcatctgtagtctacttcctagagtgtctctgaaaacactcaagctgtaaatttgcactcagcacagcccttctgtttctcaagaactagccatgggttgttagtatcagagatcccagtgtgtcagttctaaaataccctcacaagggttccagacgaggaaggaggcctgctcagcagaatagtaggtggtttccatctaagcagtgagccatcgatccccaggttctggtctcatttgccaagagggttgatatctggtttttccttgacagtgcacattcgaaggctgcgggaagcgcttttcactggacttcaatttgcgcacgcatgtgcgaatccataccggagacaggccctatgtgtgccccttcgacggttgtaataagaagtttgctcagtcaactaacctgaaatctcacatcttaacacacgctaaagccaaaaacaaccagtgaaaagaagagagaagaccttctcgaccccgggaagcctcttcaggagtgtgattgggaataaatatgcctctcctttgtatattatttctaggaagaattttaaaaatgaatcctacacacttaagggacatgttttgataaagtagtaaaaatttaaaaaaatactttaataagatgacattgctaagatgctctatcttgctctgtaatctcgtttcaaaaacaaggtgtttttgtaaagtgtggtcccaacaggaggacaattcatgaacttcgcatcaaaagacaattctttatacaacagtgctaaaaatg

SEQ ID NO: 4 - protein, 411 aa, Rattus norv.
  misc_feature (955)..(1125): Zinc finger
  misc_feature (1759)..(1917): Zinc finger
MASGDTLYIATDGSEMPAEIVELHEIEVETIPVETIETTVVGEEEDDDEDDEDGGGGDHGGGGGHGHAGHHHHHHHHHHPPMIALQPLVTDDPTQVHHHQEVILVQTREEVVGGDDSDGLRAEDGFEDQILIPVPAPAGGDDDYIEQTLVTVAAAGKSGGGSSSGGGRVKKGGGKKSGKKSYLGSGAGAAGGGGADPGNKKWEQKQVQIKTLEGEFSVTMWSSDEKKDIDHETVVEEQIIGENSPPDYSEYMTGKKLPPGGIPGIDLSDPKQLAEFARMKPRKIKEDDAPRTIACPHKGCTKRFRDNSAMKKHLHTHGPRVHVCAECGKAFVESSKLKRHQLVHTGEKPFQCTFEGCGKRFSLDFNLRTHVRIHTGDRPYVCPFDGCNKKFAQSTNLKSHILTHAKAKNNQ

SEQ ID NO: 5 - DNA, 1600 bp, Homo sapiens
  CDS (43)..(1284): YY1 (Human)
gaattcggcacgagggcggccgtggcggcggagccctcagccatggcctcgggcgacaccctctacatcgccacggacggctcggagatgccggccgagatcgtggagctgcatgagatcgaggtggagaccatcccggtggagaccatcgagaccacggtggtgggcgaggaggaggaggaggacgacgacgacgaggacggcggcggcggcgaccacggcggcggcgggggcggccacgggcacgccggccaccaccatcaccaccaccaccaccaccaccaccacccgcccatgatcgcgctggagccgctggtgacggacgacccgacccaagtgcaccacctccaggaggtgatcctggtgcagacgcgcgaggaggtcgtcgggggggacgactcggacgggctgcgcgccgaggacggcttcgaggacgagatcctcatcccggtgcccgcgccggccggcggcgacgacgactacatagagcagacgctggtcaccgtggcggcggccggcaagagcggcggcggggcctcgtcgggcggcggtcgcgtgaagaagggcggcggcaagaagagcggcaagaagagttacctgggcggcggggccggcgcggcgggcggcggcggcgccgacccggggaataagaagtgggagcagaagcaggtgcagatcaagaccctggagggcgagtcctcggtcaccatgtggtcctcggatgaaaaaaaagatattgaccatgaaacagtggttgaagagcagatcattggagagaactcacctcctgattattctgaatatatgacaggcaagaaactccctcctggagggatacctggcattgacctctcagaccctaagcaactggcagaatttgccagaatgaagccaagaaaaattaaagaagatgatgctccaagaacaatagcttgccctcataaaggctgcacaaagatgttcagggataactctgctatgagaaagcatctgcacacccacggtcccagagtccacgtctgtgcagagtgtggcaaagcgttcgttgagagctcaaagctaaaacgacaccagctggttcatactggagaaaagccctttcagtgcacattcgaaggctgcgggaagcgcttttcactggacttcaatttgcgcacacatgtgggaatccataccggagacaggccctatgtgtgccccttcgacggttgtaataagaagtttgctcagtcaactaacctgaaatctcacatcttaacacacgctaaagccaaaaacaaccagtgaaaagaagagagaagaccttctcgacccgggaagcctcttcaggagtgagattgggaataaatatgcctctcctttgtatattatttctaggaagaattttaaaaatgaatcctacacacttaagggacatgttttgataaagtagtaaaaatttaaaaaatactttaataagatgacattgctaagatgctatatcttgctctgtaatctcgtttcaaaaacaaggtgtttttgtaaagtgtggtcccaacaggaggacaattcatgaacttcgcatcaaaagacaattctttatacaacagtgctaaaaatg

SEQ ID NO: 6 - protein, 414 aa, Homo sapiens
MASGDTLYIATDGSEMPAEIVELHEIEVETIPVETIETTVVGEEEEEDDDDEDGGGGDHGGGGGGHGHAGHHHHHHHHHHHHPPMIALEPLVTDDPTQVHHLQEVILVQTREEVVGGDDSDGLRAEDGFEDEILIPVPAPAGGDDDYIEQTLVTVAAAGKSGGGASSGGGRVKKGGGKKSGKKSYLGGGAGAAGGGGADPGNKKWEQKQVQIKTLEGESSVTMWSSDEKKDIDHETVVEEQIIGENSPPDYSEYMTGKKLPPGGIPGIDLSDPKQLAEFARMKPRKIKEDDAPRTIACPHKGCTKMFRDNSAMRKHLHTHGPRVHVCAECGKAFVESSKLKRHQLVHTGEKPFQCTFEGCGKRFSLDFNLRTHVGIHTGDRPYVCPFDGCNKKFAQSTNLKSHILTHAKAKNNQ

SEQ ID NO: 7 - DNA, 1080 bp, Rattus norv.
  CDS (883)..(894): Zinc finger
  CDS (898)..(1056): Zinc finger
atggcctcgggcgacaccctctacattgccacggacggctcggagatgccagccgagatcgtggaactgcatgagattgaggtggagaccatcccggtggagactatcgagaccacggtggtgggcgaggaggaggacgacgacgaagacgacgaggatggtggcggcggagaccacggtggcgggggcggccacgggcacgctggccaccaccatcaccaccaccaccaccaccacccgcccatgatcgcgctgcagccgctggtcaccgacgacccgacccaagtgcaccaccaccaagaggtgattctggtgcagacgcgcgaggaggtagtgggtggcgacgactcggacgggctgcgcgccgaggacgggttcgaggaccagatcctcattccggtacccgcgccggccggcggagacgacgactacatcgagcagacgctggtcaccgtggcggcggccggcaagagcggtggcgggtcttcgtcgggcggcggccgcgttaagaagggcggcggcaagaagagcggcaagaagagttacctgggcagcggggccggcgcggcgggcggtggcggcgccgacccgggtaataagaagtgggaacagaagcaggtgcagatcaagaccctggagggcgagttctcggtcaccatgtggtcttcagatgaaaaaaaagatattgaccatgaaacagtggttgaagagcagatcattggggagaactcacctcctgattattctgaatatatgacaggcaagaaactccctcctggagggatacctggcattgacctctcagaccccaagcaactggcagaatttgccagaatgaagccaagaaaaattaaagaagatgatgctccaagaacaatagcttgccctcataaacagtgcacattcgaaggctgcgggaagcgcttttcactggacttcaatttgcgcacgcatgtgcgaatccataccggagacaggccctatgtgtgccccttcgacggttgtaataagaagtttgctcagtcaactaacctgaaatctcacatcttaacacacgctaaagccaaaaacaaccagtga

SEQ ID NO: 8 - DNA, 1080 bp, Rattus norv.; identical to SEQ ID NO: 7

SEQ ID NO: 9 - protein, 57 aa, Rattus norv.
CPHKCTFEGCGKRFSLDFNLRTHVRIHTGDRPYVCPFDGCNKKFAQSTNLKSHILTH

SEQ ID NO: 10 - DNA, 21 bp, Artificial sequence
  Synthetic oligonucleotide primer
cacaggcgtttctcgtcagag

SEQ ID NO: 11 - DNA, 22 bp, Artificial sequence
  Synthetic oligonucleotide primer
aataccaactcctcaaccccga

SEQ ID NO: 12 - DNA, 21 bp, Artificial sequence
  Synthetic oligonucleotide primer
cttcctccctctgccttcctt

SEQ ID NO: 13 - DNA, 21 bp, Artificial sequence
  Synthetic oligonucleotide primer
gagatcgtggaactgcatgag

SEQ ID NO: 14 - DNA, 24 bp, Artificial sequence
  Synthetic oligonucleotide primer
gtcttcgtcgtcgtcctcctcctc

SEQ ID NO: 15 - DNA, 21 bp, Artificial sequence
  Synthetic oligonucleotide primer
cggagacgacgactacatcga

SEQ ID NO: 16 - DNA, 25 bp, Artificial sequence
  Synthetic oligonucleotide primer
gtgaccagcgtctgctcgatgtagt

SEQ ID NO: 17 - DNA, 21 bp, Artificial sequence
  Synthetic oligonucleotide primer
ccaggtaactcttcttgccgc

SEQ ID NO: 18 - DNA, 22 bp, Artificial sequence
  Synthetic oligonucleotide primer
gttcccacttcttattacccgg

SEQ ID NO: 19 - DNA, 22 bp, Artificial sequence
  Synthetic oligonucleotide primer
caagaccctggagggcgagttc

SEQ ID NO: 20 - DNA, 25 bp, Artificial sequence
  Synthetic oligonucleotide primer
acagtggttgaagagcagatcattg

SEQ ID NO: 21 - DNA, 23 bp, Artificial sequence
  Synthetic oligonucleotide primer
ccaatgatctgctcttcaaccac

SEQ ID NO: 22 - DNA, 27 bp, Artificial sequence
  Synthetic oligonucleotide primer
gccaagaaaaattaaagaagatgatgc

SEQ ID NO: 23 - DNA, 26 bp, Artificial sequence
  Synthetic oligonucleotide primer
gctattgttcttggagcatcatcttc

SEQ ID NO: 24 - DNA, 25 bp, Artificial sequence
  Synthetic oligonucleotide primer
gagagctcaaagctaaaacgacacc

SEQ ID NO: 25 - DNA, 25 bp, Artificial sequence
  Synthetic oligonucleotide primer
aaagggcttttctccagtatgaacc

SEQ ID NO: 26 - DNA, 23 bp, Artificial sequence
  Synthetic oligonucleotide primer
aattgaagtccagtgaaaagggc

SEQ ID NO: 27 - DNA, 22 bp, Artificial sequence
  Synthetic oligonucleotide primer
acgcatgtgcgaatccataccg

SEQ ID NO: 28 - DNA, 27 bp, Artificial sequence
  Synthetic oligonucleotide primer
caaaacatgtcccttaagtgtgtagga

SEQ ID NO: 29 - DNA, 27 bp, Artificial sequence
  Synthetic oligonucleotide primer
aattgtaagcaacaggtgagcttcatg

SEQ ID NO: 30 - DNA, 25 bp, Artificial sequence
  Synthetic oligonucleotide primer
gcgaagcacccccacactaaatttc

SEQ ID NO: 31 - DNA, 26 bp, Artificial sequence
  Synthetic oligonucleotide primer
gcttataagtgctgttggctacagct

SEQ ID NO: 32 - DNA, 22 bp, Artificial sequence
  Synthetic oligonucleotide primer
gtcacctggagctgtagccaac

SEQ ID NO: 33 - DNA, 21 bp, Artificial sequence
  Synthetic oligonucleotide primer
tcactggacttcaatttgcgc

SEQ ID NO: 34 - DNA, 23 bp, Artificial sequence
  Synthetic oligonucleotide primer
ttttcactggacttcaatttgcg

SEQ ID NO: 35 - DNA, 22 bp, Artificial sequence
  Synthetic oligonucleotide primer
accagatcctcattccggtacc

SEQ ID NO: 36 - DNA, 21 bp, Artificial sequence
  Synthetic oligonucleotide primer
ccctttcagtgcacattcgaa

SEQ ID NO: 37 - DNA, 21 bp, Artificial sequence
  Synthetic oligonucleotide primer
gacgacgaagacgacgaggat

SEQ ID NO: 38 - DNA, 25 bp, Artificial sequence
  Synthetic oligonucleotide primer
gagagctcaaagctaaaacgacacc

SEQ ID NO: 39 - DNA, 22 bp, Artificial sequence
  Synthetic oligonucleotide primer
ggagacgacgactacatcgagc

SEQ ID NO: 40 - DNA, 21 bp, Artificial sequence
  Synthetic oligonucleotide primer
cggagacgacgactacatcga

SEQ ID NO: 41 - DNA, 25 bp, Artificial sequence
  Synthetic oligonucleotide primer
tgagagctcaaagctaaaacgacac

SEQ ID NO: 42 - DNA, 21 bp, Artificial sequence
  Synthetic oligonucleotide primer
gaggaccagatcctcattccg

SEQ ID NO: 43 - DNA, 22 bp, Artificial sequence
  Synthetic oligonucleotide primer
aactccctcctggagggatacc

SEQ ID NO: 44 - DNA, 23 bp, Artificial sequence
  Synthetic oligonucleotide primer
gagacgacgactacatcgagcag

SEQ ID NO: 45 - DNA, 21 bp, Artificial sequence
  Synthetic oligonucleotide primer
gaggaggacgacgacgaagac

SEQ ID NO: 46 - DNA, 25 bp, Artificial sequence
  Synthetic oligonucleotide primer
ttgagagctcaaagctaaaacgaca

SEQ ID NO: 47 - DNA, 21 bp, Artificial sequence
  Synthetic oligonucleotide primer
accctctacattgccacggac

SEQ ID NO: 48 - DNA, 22 bp, Artificial sequence
  Synthetic oligonucleotide primer
actacatcgagcagacgctggt

SEQ ID NO: 49 - DNA, 24 bp, Artificial sequence
  Synthetic oligonucleotide primer
gagctcaaagctaaaacgacacca

SEQ ID NO: 50 - DNA, 21 bp, Artificial sequence
  Synthetic oligonucleotide primer
ttcagtgcacattcgaaggct

SEQ ID NO: 51 - DNA, 22 bp, Artificial sequence
  Synthetic oligonucleotide primer
tggagactatcgagaccacggt

SEQ ID NO: 52 - DNA, 21 bp, Artificial sequence
  Synthetic oligonucleotide primer
tttcagtgcacattcgaaggc

SEQ ID NO: 53 - DNA, 21 bp, Artificial sequence
  Synthetic oligonucleotide primer
gtgcgaatccataccggagac

SEQ ID NO: 54 - DNA, 21 bp, Artificial sequence
  Synthetic oligonucleotide primer
gaggtgattctggtgcagacg

SEQ ID NO: 55 - DNA, 22 bp, Artificial sequence
  Synthetic oligonucleotide primer
actccctcctggagggatacct

SEQ ID NO: 56 - DNA, 22 bp, Artificial sequence
  Synthetic oligonucleotide primer
gtggagactatcgagaccacgg

SEQ ID NO: 57 - DNA, 22 bp, Artificial sequence
  Synthetic oligonucleotide primer
agaggtgattctggtgcagacg

SEQ ID NO: 58 - DNA, 26 bp, Artificial sequence
  Synthetic oligonucleotide primer
tgaaatctcacatcttaacacacgct

SEQ ID NO: 59 - DNA, 21 bp, Artificial sequence
  Synthetic oligonucleotide primer
tacatcgagcagacgctggtc

SEQ ID NO: 60 - DNA, 22 bp, Artificial sequence
  Synthetic oligonucleotide primer
acgactacatcgagcagacgct

SEQ ID NO: 61 - DNA, 23 bp, Artificial sequence
  Synthetic oligonucleotide primer
gaaactccctcctggagggatac

SEQ ID NO: 62 - DNA, 25 bp, Artificial sequence
  Synthetic oligonucleotide primer
ctgcacaaagatgttcagggataac

SEQ ID NO: 63 - DNA, 24 bp, Artificial sequence
  Synthetic oligonucleotide primer
aaaacgacaccagctggttcatac

SEQ ID NO: 64 - DNA, 25 bp, Artificial sequence
  Synthetic oligonucleotide primer
taaaacgacaccagctggttcatac

SEQ ID NO: 65 - DNA, 24 bp, Artificial sequence
  Synthetic oligonucleotide primer
agaagagcggcaagaagagttacc

SEQ ID NO: 66 - DNA, 27 bp, Artificial sequence
  Synthetic oligonucleotide primer
acctgaaatctcacatcttaacacacg

SEQ ID NO: 67 - DNA, 26 bp, Artificial sequence
  Synthetic oligonucleotide primer
cctgaaatctcacatcttaacacacg

SEQ ID NO: 68 - DNA, 23 bp, Artificial sequence
  Synthetic oligonucleotide primer
gacaccagctggttcatactgga

SEQ ID NO: 69 - DNA, 22 bp, Artificial sequence
  Synthetic oligonucleotide primer
ggtggagactatcgagaccacg

SEQ ID NO: 70 - DNA, 23 bp, Artificial sequence
  Synthetic oligonucleotide primer
agacgacgactacatcgagcaga

SEQ ID NO: 71 - DNA, 24 bp, Artificial sequence
  Synthetic oligonucleotide primer
cagtggttgaagagcagatcattg

SEQ ID NO: 72 - DNA, 25 bp, Artificial sequence
  Synthetic oligonucleotide primer
acagtggttgaagagcagatcattg

SEQ ID NO: 73 - DNA, 22 bp, Artificial sequence
  Synthetic oligonucleotide primer
ggttgaagagcagatcattggg

SEQ ID NO: 74 - DNA, 21 bp, Artificial sequence
  Synthetic oligonucleotide primer
ggtcccagagtccacgtctgt

SEQ ID NO: 75 - DNA, 25 bp, Artificial sequence
  Synthetic oligonucleotide primer
tgcacaaagatgttcagggataact

SEQ ID NO: 76 - DNA, 24 bp, Artificial sequence
  Synthetic oligonucleotide primer
gatgctccaagaacaatagcttgc

SEQ ID NO: 77 - DNA, 21 bp, Artificial sequence
  Synthetic oligonucleotide primer
gtcccagagtccacgtctgtg

SEQ ID NO: 78 - DNA, 24 bp, Artificial sequence
  Synthetic oligonucleotide primer
gcttttcactggacttcaatttgc

SEQ ID NO: 79 - DNA, 24 bp, Artificial sequence
  Synthetic oligonucleotide primer
agtggttgaagagcagatcattgg

SEQ ID NO: 80 - DNA, 23 bp, Artificial sequence
  Synthetic oligonucleotide primer
gtggttgaagagcagatcattgg

SEQ ID NO: 81 - DNA, 23 bp, Artificial sequence
  Synthetic oligonucleotide primer
agagcggcaagaagagttacctg

SEQ ID NO: 82 - DNA, 25 bp, Artificial sequence
  Synthetic oligonucleotide primer
tcacatcttaacacacgctaaagcc

SEQ ID NO: 83 - DNA, 27 bp, Artificial sequence
  Synthetic oligonucleotide primer
atctcacatcttaacacacgctaaagc

SEQ ID NO: 84 - DNA, 26 bp, Artificial sequence
  Synthetic oligonucleotide primer
ctgaaatctcacatcttaacacacgc

SEQ ID NO: 85 - DNA, 23 bp, Artificial sequence
  Synthetic oligonucleotie primer
acgacaccagctggttcatactg

SEQ ID NO: 86 - DNA, 27 bp, Artificial sequence
  Synthetic oligonucleotide primer
agatattgaccatgaaacagtggttga

SEQ ID NO: 87 - DNA, 26 bp, Artificial sequence
  Synthetic oligonucleotide primer
gatattgaccatgaaacagtggttga

SEQ ID NO: 88 - DNA, 22 bp, Artificial sequence
  Synthetic oligonucleotide primer
gagggatacctggcattgacct

SEQ ID NO: 89 - DNA, 22 bp, Artificial sequence
  Synthetic oligonucleotide primer
agaccatcccggtggagactat

SEQ ID NO: 90 - DNA, 24 bp, Artificial sequence
  Synthetic oligonucleotide primer
gaagagcggcaagaagagttacct

SEQ ID NO: 91 - DNA, 22 bp, Artificial sequence
  Synthetic oligonucleotide primer
ggagactatcgagaccacggtg

SEQ ID NO: 92 - DNA, 21 bp, Artificial sequence
  Synthetic oligonucleotide primer
ggttcgaggaccagatcctca

SEQ ID NO: 93 - DNA, 23 bp, Artificial sequence
  Synthetic oligonucleotide primer
gagcagatcattggggagaactc

SEQ ID NO: 94 - DNA, 26 bp, Artificial sequence
  Synthetic oligonucleotide primer
gaagatgatgctccaagaacaatagc

SEQ ID NO: 95 - DNA, 23 bp, Artificial sequence
  Synthetic oligonucleotide primer
cgctaaagccaaaaacaaccagt

SEQ ID NO: 96 - DNA, 22 bp, Artificial sequence
  Synthetic oligonucleotide primer
ataccggagacaggccctatgt

SEQ ID NO: 97 - DNA, 23 bp, Artificial sequence
  Synthetic oligonucleotide primer
caatagcttgccctcataaaggc

SEQ ID NO: 98 - DNA, 27 bp, Artificial sequence
  Synthetic oligonucleotide primer
aagatattgaccatgaaacagtggttg

SEQ ID NO: 99 - DNA, 24 bp, Artificial sequence
  Synthetic oligonucleotide primer
acaatagcttgccctcataaaggc

SEQ ID NO: 100 - DNA, 22 bp, Artificial sequence
  Synthetic oligonucleotide primer
agaaaagccctttcagtgcaca

SEQ ID NO: 101 - DNA, 27 bp, Artificial sequence
  Synthetic oligonucleotide primer
atattgaccatgaaacagtggttgaag

SEQ ID NO: 102 - DNA, 21 bp, Artificial sequence
  Synthetic oligonucleotide primer
gcggcaagaagagttacctgg

SEQ ID NO: 103 - DNA, 26 bp, Artificial sequence
  Synthetic oligonucleotide primer
tattgaccatgaaacagtggttgaag

SEQ ID NO: 104 - DNA, 25 bp, Artificial sequence
  Synthetic oligonucleotide primer
attgaccatgaaacagtggttgaag

SEQ ID NO: 105 - DNA, 24 bp, Artificial sequence
  Synthetic oligonucleotide primer
ttgaccatgaaacagtggttgaag

SEQ ID NO: 106 - DNA, 25 bp, Artificial sequence
  Synthetic oligonucleotide primer
gaacaatagcttgccctcataaagg

SEQ ID NO: 107 - DNA, 26 bp, Artificial sequence
  Synthetic oligonucleotide primer
agaacaatagcttgccctcataaagg

SEQ ID NO: 108 - DNA, 22 bp, Artificial sequence
  Synthetic oligonucleotide primer
acctctcagaccccaagcaact

SEQ ID NO: 109 - DNA, 23 bp, Artificial sequence
  Synthetic oligonucleotide primer
acgctaaagccaaaaacaaccag

SEQ ID NO: 110 - DNA, 27 bp, Artificial sequence
  Synthetic oligonucleotide primer
aagatgatgctccaagaacaatagctt

SEQ ID NO: 111 - DNA, 24 bp, Artificial sequence
  Synthetic oligonucleotide primer
aaacgacaccagctggttcatact

SEQ ID NO: 112 - DNA, 26 bp, Artificial sequence
  Synthetic oligonucleotide primer
cgacggttgtaataagaagtttgctc

SEQ ID NO: 113 - DNA, 26 bp, Artificial sequence
  Synthetic oligonucleotide primer
caagaacaatagcttgccctcataaa

SEQ ID NO: 114 - DNA, 22 bp, Artificial sequence
  Synthetic oligonucleotide primer
ggaacagaagcaggtgcagatc

SEQ ID NO: 115 - DNA, 23 bp, Artificial sequence
  Synthetic oligonucleotide primer
aaaagccctttcagtgcacattc

SEQ ID NO: 116 - DNA, 24 bp, Artificial sequence
  Synthetic oligonucleotide primer
tctgctatgagaaagcatctgcac

SEQ ID NO: 117 - DNA, 26 bp, Artificial sequence
  Synthetic oligonucleotide primer
aaacagtggttgaagagcagatcatt

SEQ ID NO: 118 - DNA, 26 bp, Artificial sequence
  Synthetic oligonucleotide primer
ttcgacggttgtaataagaagtttgc

SEQ ID NO: 119 - DNA, 22 bp, Artificial sequence
  Synthetic oligonucleotide primer
agcgttcgttgagagctcaaag

SEQ ID NO: 120 - DNA, 21 bp, Artificial sequence
  Synthetic oligonucleotide primer
gccccttcgacggttgtaata

SEQ ID NO: 121 - DNA, 21 bp, Artificial sequence
  Synthetic oligonucleotide primer
caatgcgcagaatttgccaga

SEQ ID NO: 122 - DNA, 22 bp, Artificial sequence
  Synthetic oligonucleotide primer
agttctcggtcaccatgtggtc

SEQ ID NO: 123 - DNA, 21 bp, Artificial sequence
  Synthetic oligonucleotide primer
tgagaaagcatctgcacaccc

SEQ ID NO: 124 - DNA, 22 bp, Artificial sequence
  Synthetic oligonucleotide primer
atgagaaagcatctgcacaccc

SEQ ID NO: 125 - DNA, 23 bp, Artificial sequence
  Synthetic oligonucleotide primer
tatgagaaagcatctgcacaccc

SEQ ID NO: 126 - DNA, 22 bp, Artificial sequence
  Synthetic oligonucleotide primer
gagttctcggtcaccatgtggt

SEQ ID NO: 127 - DNA, 22 bp, Artificial sequence
  Synthetic oligonucleotide primer
caccaccaccaagaggtgattc

SEQ ID NO: 128 - DNA, 23 bp, Artificial sequence
  Synthetic oligonucleotide primer
gacgacgactacatcgagcagac

SEQ ID NO: 129 - DNA, 21 bp, Artificial sequence
  Synthetic oligonucleotide primer
cccggtggagactatcgagac

SEQ ID NO: 130 - DNA, 23 bp, Artificial sequence
  Synthetic oligonucleotide primer
cagaagcaggtgcagatcaagac

SEQ ID NO: 131 - DNA, 24 bp, Artificial sequence
  Synthetic oligonucleotide primer
gctaaagccaaaaacaaccagtga

SEQ ID NO: 132 - DNA, 21 bp, Artificial sequence
  Synthetic oligonucleotide primer
gacctctcagaccccaagcaa

SEQ ID NO: 133 - DNA, 26 bp, Artificial sequence
  Synthetic oligonucleotide primer
gcaaacttcttattacaaccgtcgaa

SEQ ID NO: 134 - DNA, 22 bp, Artificial sequence
  Synthetic oligonucleotide primer
acatagggcctgtctccggtat

SEQ ID NO: 135 - DNA, 26 bp, Artificial sequence
  Synthetic oligonucleotide primer
agcaaacttcttattacaaccgtcga

SEQ ID NO: 136 - DNA, 22 bp, Artificial sequence
  Synthetic oligonucleotide primer
agctttgagctctcaacgaacg

SEQ ID NO: 137 - DNA, 26 bp, Artificial sequence
  Synthetic oligonucleotide primer
gagcaaacttcttattacaaccgtcg

SEQ ID NO: 138 - DNA, 22 bp, Artificial sequence
  Synthetic oligonucleotide primer
ctttgagctctcaacgaacgct

SEQ ID NO: 139 - DNA, 23 bp, Artificial sequence
  Synthetic oligonucleotide primer
ggttgtttttggctttagcgtgt

SEQ ID NO: 140 - DNA, 23 bp, Artificial sequence
  Synthetic oligonucleotide primer
ctggttgtttttggctttagcgt

SEQ ID NO: 141 - DNA, 21 bp, Artificial sequence
  Synthetic oligonucleotide primer
cctgtctccggtatggattcg

SEQ ID NO: 142 - DNA, 21 bp, Artificial sequence
  Synthetic oligonucleotide primer
ctgtctccggtatggattcgc

SEQ ID NO: 143 - DNA, 21 bp, Artificial sequence
  Synthetic oligonucleotide primer
gtctccggtatggattcgcac

SEQ ID NO: 144 - DNA, 22 bp, Artificial sequence
  Synthetic oligonucleotide primer
agcgtctgctcgatgtagtcgt

SEQ ID NO: 145 - DNA, 25 bp, Artificial sequence
  Synthetic oligonucleotide primer
ttctgttcccacttcttattacccg

SEQ ID NO: 146 - DNA, 23 bp, Artificial sequence
  Synthetic oligonucleotide primer
tctgctcgatgtagtcgtcgtct

SEQ ID NO: 147 - DNA, 23 bp, Artificial sequence
  Synthetic oligonucleotide primer
actggttgtttttggctttagcg

SEQ ID NO: 148 - DNA, 23 bp, Artificial sequence
  Synthetic oligonucleotide primer
gtctgctcgatgtagtcgtcgtc

SEQ ID NO: 149 - DNA, 21 bp, Artificial sequence
  Synthetic oligonucleotide primer
atcctcgtcgtcttcgtcgtc

SEQ ID NO: 150 - DNA, 23 bp, Artificial sequence
  Synthetic oligonucleotide primer
cagtatgaaccagctggtgtcgt

SEQ ID NO: 151 - DNA, 22 bp, Artificial sequence
  Synthetic oligonucleotide primer
ttgagctctcaacgaacgcttt

SEQ ID NO: 152 - DNA, 22 bp, Artificial sequence
  Synthetic oligonucleotide primer
agaccacatggtgaccgagaac

SEQ ID NO: 153 - DNA, 21 bp, Artificial sequence
  Synthetic oligonucleotide primer
cttcttattacccgggtcggc

SEQ ID NO: 154 - DNA, 23 bp, Artificial sequence
  Synthetic oligonucleotide primer
ctgctcgatgtagtcgtcgtctc

SEQ ID NO: 155 - DNA, 21 bp, Artificial sequence
  Synthetic oligonucleotide primer
tcgatgtagtcgtcgtctccg

SEQ ID NO: 156 - DNA, 22 bp, Artificial sequence
  Synthetic oligonucleotide primer
tttgagctctcaacgaacgctt

SEQ ID NO: 157 - DNA, 22 bp, Artificial sequence
  Synthetic oligonucleotide primer
ccacttcttattacccgggtcg

SEQ ID NO: 158 - DNA, 22 bp, Artificial sequence
  Synthetic oligonucleotide primer
cacttcttattacccgggtcgg

SEQ ID NO: 159 - DNA, 21 bp, Artificial sequence
  Synthetic oligonucleotide primer
gaccagcgtctgctcgatgta

SEQ ID NO: 160 - DNA, 23 bp, Artificial sequence
  Synthetic oligonucleotide primer
aattgaagtccagtgaaaagcgc

SEQ ID NO: 161 - DNA, 23 bp, Artificial sequence
  Synthetic oligonucleotide primer
tgaaccagctggtgtcgttttag

SEQ ID NO: 162 - DNA, 22 bp, Artificial sequence
  Synthetic oligonucleotide primer
gaccacatggtgaccgagaact

SEQ ID NO: 163 - DNA, 26 bp, Artificial sequence
  Synthetic oligonucleotide primer
aacttcttattacaaccgtcgaaggg

SEQ ID NO: 164 - DNA, 23 bp, Artificial sequence
  Synthetic oligonucleotide primer
tgttcccacttcttattacccgg

SEQ ID NO: 165 - DNA, 21 bp, Artificial sequence
  Synthetic oligonucleotide primer
cccaggtaactcttcttgccg

SEQ ID NO: 166 - DNA, 22 bp, Artificial sequence
  Synthetic oligonucleotide primer
agaggtcaatgccaggtatccc

SEQ ID NO: 167 - DNA, 21 bp, Artificial sequence
  Synthetic oligonucleotide primer
ccaggtaactcttcttgccgc

SEQ ID NO: 168 - DNA, 22 bp, Artificial sequence
  Synthetic oligonucleotide primer
ttgaagtccagtgaaaagcgct

SEQ ID NO: 169 - DNA, 21 bp, Artificial sequence
  Synthetic oligonucleotide primer
tgaggatctggtcctcgaacc

SEQ ID NO: 170 - DNA, 21 bp, Artificial sequence
  Synthetic oligonucleotide primer
cacatggtgaccgagaactcg

SEQ ID NO: 171 - DNA, 24 bp, Artificial sequence
  Synthetic oligonucleotide primer
gtatgaaccagctggtgtcgtttt

SEQ ID NO: 172 - DNA, 23 bp, Artificial sequence
  Synthetic oligonucleotide primer
tcaatctcatgcagttccacgat

SEQ ID NO: 173 - DNA, 22 bp, Artificial sequence
  Synthetic oligonucleotide primer
tcaatctcatgcagttccacga

SEQ ID NO: 174 - DNA, 24 bp, Artificial sequence
  Synthetic oligonucleotide primer
agtatgaaccagctggtgtcgttt

SEQ ID NO: 175 - DNA, 21 bp, Artificial sequence
  Synthetic oligonucleotide primer
ggtctcgatagtctccaccgg

SEQ ID NO: 176 - DNA, 22 bp, Artificial sequence
  Synthetic oligonucleotide primer
aagaccacatggtgaccgagaa

SEQ ID NO: 177 - DNA, 23 bp, Artificial sequence
  Synthetic oligonucleotide primer
caatctcatgcagttccacgatc

SEQ ID NO: 178 - DNA, 21 bp, Artificial sequence
  Synthetic oligonucleotide primer
ggaatgaggatctggtcctcg

SEQ ID NO: 179 - DNA, 23 bp, Artificial sequence
  Synthetic oligonucleotide primer
ttcccacttcttattacccgggt

SEQ ID NO: 180 - DNA, 22 bp, Artificial sequence
  Synthetic oligonucleotide primer
tgaagtccagtgaaaagcgctt

SEQ ID NO: 181 - DNA, 22 bp, Artificial sequence
  Synthetic oligonucleotide primer
gctcgatgtagtcgtcgtctcc

SEQ ID NO: 182 - DNA, 25 bp, Artificial sequence
  Synthetic oligonucleotide primer
gtatgaaccagctggtgtcgtttta

SEQ ID NO: 183 - DNA, 22 bp, Artificial sequence
  Synthetic oligonucleotide primer
ttcccacttcttattacccggg

SEQ ID NO: 184 - DNA, 23 bp, Artificial sequence
  Synthetic oligonucleotide primer
gaatgaggatctggtcctcgaac

SEQ ID NO: 185 - DNA, 22 bp, Artificial sequence
  Synthetic oligonucleotide primer
gaggtcaatgccaggtatccct

SEQ ID NO: 186 - DNA, 22 bp, Artificial sequence
  Synthetic oligonucleotide primer
gtggtctcgatagtctccaccg

SEQ ID NO: 187 - DNA, 24 bp, Artificial sequence
  Synthetic oligonucleotide primer
aggtaactcttcttgccgctcttc

SEQ ID NO: 188 - DNA, 21 bp, Artificial sequence
  Synthetic oligonucleotide primer
cacattctgcacagacgtgga

SEQ ID NO: 189 - DNA, 25 bp, Artificial sequence
  Synthetic oligonucleotide primer
aaagggcttttctccagtatgaacc

SEQ ID NO: 190 - DNA, 21 bp, Artificial sequence
  Synthetic oligonucleotide primer
accatcctcgtcgtcttcgtc

SEQ ID NO: 191 - DNA, 26 bp, Artificial sequence
  Synthetic oligonucleotide primer
gcttctgttcccacttcttattaccc

SEQ ID NO: 192 - DNA, 22 bp, Artificial sequence
  Synthetic oligonucleotide primer
cacattctgcacagacgtggac

SEQ ID NO: 193 - DNA, 23 bp, Artificial sequence
  Synthetic oligonucleotide primer
caggtaactcttcttgccgctct

SEQ ID NO: 194 - DNA, 27 bp, Artificial sequence
  Synthetic oligonucleotide primer
gatgctttctcatagcagagttatccc

SEQ ID NO: 195 - DNA, 21 bp, Artificial sequence
  Synthetic oligonucleotide primer
ctgaagaccacatggtgaccg

SEQ ID NO: 196 - DNA, 27 bp, Artificial sequence
  Synthetic oligonucleotide primer
cctgcttctgttcccacttcttattac

SEQ ID NO: 197 - DNA, 22 bp, Artificial sequence
  Synthetic oligonucleotide primer
accagcgtctgctcgatgtagt

SEQ ID NO: 198 - DNA, 23 bp, Artificial sequence
  Synthetic oligonucleotide primer
tcttattacaaccgtcgaagggg

SEQ ID NO: 199 - DNA, 24 bp, Artificial sequence
  Synthetic oligonucleotide primer
ttgtttttggctttagcgtgtgtt

SEQ ID NO: 200 - DNA, 25 bp, Artificial sequence
  Synthetic oligonucleotide primer
actgaaagggcttttctccagtatg

SEQ ID NO: 201 - DNA, 25 bp, Artificial sequence
  Synthetic oligonucleotide primer
cactgaaagggcttttctccagtat

SEQ ID NO: 202 - DNA, 23 bp, Artificial sequence
  Synthetic oligonucleotide primer
gaggtgagttctccccaatgatc

SEQ ID NO: 203 - DNA, 22 bp, Artificial sequence
  Synthetic oligonucleotide primer
ggtaccggaatgaggatctggt

SEQ ID NO: 204 - DNA, 21 bp, Artificial sequence
  Synthetic oligonucleotide primer
gtctcgatagtctccaccggg

SEQ ID NO: 205 - DNA, 26 bp, Artificial sequence
  Synthetic oligonucleotide primer
cttcaaccactgtttcatggtcaata

SEQ ID NO: 206 - DNA, 25 bp, Artificial sequence
  Synthetic oligonucleotide primer
cctttatgagggcaagctattgttc

SEQ ID NO: 207 - DNA, 27 bp, Artificial sequence
  Synthetic oligonucleotide primer
cttcaaccactgtttcatggtcaatat

SEQ ID NO: 208 - DNA, 26 bp, Artificial sequence
  Synthetic oligonucleotide primer
tttttggctttagcgtgtgttaagat

SEQ ID NO: 209 - DNA, 25 bp, Artificial sequence
  Synthetic oligonucleotide primer
ttgtttttggctttagcgtgtgtta

SEQ ID NO: 210 - DNA, 22 bp, Artificial sequence
  Synthetic oligonucleotide primer
cttggggtctgagaggtcaatg

SEQ ID NO: 211 - DNA, 21 bp, Artificial sequence
  Synthetic oligonucleotide primer
gtccgtggcaatgtagagggt

SEQ ID NO: 212 - DNA, 21 bp, Artificial sequence
  Synthetic oligonucleotide primer
tctggcaaattctgccagttg

SEQ ID NO: 213 - DNA, 24 bp, Artificial sequence
  Synthetic oligonucleotide primer
tcactggttgtttttggctttagc

SEQ ID NO: 214 - DNA, 22 bp, Artificial sequence
  Synthetic oligonucleotide primer
ctttgtgcagcctttatgaggg

SEQ ID NO: 215 - DNA, 24 bp, Artificial sequence
  Synthetic oligonucleotide primer
gttgtttttggctttagcgtgtgt

SEQ ID NO: 216 - DNA, 24 bp, Artificial sequence
  Synthetic oligonucleotide primer
tgaaagggcttttctccagtatga

SEQ ID NO: 217 - DNA, 24 bp, Artificial sequence
  Synthetic oligonucleotide primer
gcaagctattgttcttggagcatc

SEQ ID NO: 218 - DNA, 23 bp, Artificial sequence
  Synthetic oligonucleotide primer
gcctttatgagggcaagctattg

SEQ ID NO: 219 - DNA, 22 bp, Artificial sequence
  Synthetic oligonucleotide primer
gcttggggtctgagaggtcaat

SEQ ID NO: 220 - DNA, 23 bp, Artificial sequence
  Synthetic oligonucleotide primer
ccaatgatctgctcttcaaccac

SEQ ID NO: 221 - DNA, 22 bp, Artificial sequence
  Synthetic oligonucleotide primer
ccaccgtggtctcgatagtctc

SEQ ID NO: 222 - DNA, 27 bp, Artificial sequence
  Synthetic oligonucleotide primer
ctgcttctgttcccacttcttattacc

SEQ ID NO: 223 - DNA, 22 bp, Artificial sequence
  Synthetic oligonucleotide primer
ttggcttcattctggcaaattc

SEQ ID NO: 224 - DNA, 25 bp, Artificial sequence
  Synthetic oligonucleotide primer
cttcaaccactgtttcatggtcaat

SEQ ID NO: 225 - DNA, 24 bp, Artificial sequence
  Synthetic oligonucleotide primer
aatctcatgcagttccacgatctc

SEQ ID NO: 226 - DNA, 24 bp, Artificial sequence
  Synthetic oligonucleotide primer
cttcaaccactgtttcatggtcaa

SEQ ID NO: 227 - DNA, 23 bp, Artificial sequence
  Synthetic oligonucleotide primer
gggcttttctccagtatgaacca

SEQ ID NO: 228 - DNA, 22 bp, Artificial sequence
  Synthetic oligonucleotide primer
accacatggtgaccgagaactc

SEQ ID NO: 229 - DNA, 24 bp, Artificial sequence
  Synthetic oligonucleotide primer
gtgcagatgctttctcatagcaga

SEQ ID NO: 230 - DNA, 24 bp, Artificial sequence
  Synthetic oligonucleotide primer
tgtgcagatgctttctcatagcag

SEQ ID NO: 231 - DNA, 22 bp, Artificial sequence
  Synthetic oligonucleotide primer
cattctgcacagacgtggactc

SEQ ID NO: 232 - DNA, 24 bp, Artificial sequence
  Synthetic oligonucleotide primer
tctgagaggtcaatgccaggtatc